(19) United States
(12) Patent Application Publication
(10) Pub. No.: US 2026/0226181 A1
(43) Pub. Date: Aug. 6, 2026

(54) METHOD FOR TREATING AUTOIMMUNE DISEASE

(71) Applicant: THE CHILDREN'S MEDICAL CENTER CORPORATION, Boston, MA (US)

(72) Inventors: Eileen REMOLD-O'DONNELL, Brookline, MA (US); Lifei HOU, Dover, MA (US)

(21) Appl. No.: 19/301,346

(22) Filed: Aug. 15, 2025

Related U.S. Application Data
(63) Continuation of application No. 17/046,341, filed on Apr. 9, 2019, now abandoned.
(60) Provisional application No. 62/654,879, filed on Apr. 9, 2018.

(51) Int. Cl. C07K 16/28; A61K 45/06; A61P 37/00; G01N 33/68

(52) U.S. Cl. CPC C07K 16/2866; A61K 45/06; A61P 37/00; G01N 33/68; G01N 2800/24

(57) ABSTRACT

Described herein are methods and compositions for treating an autoimmune disease. Aspects of the methods described herein relate, in part, to administering to a subject an agent that targets CXCR6. Another aspect of the methods described herein relate, in part, to administering to a subject an agent that inhibits SerpinB1.

### CROSS-REFERENCE TO RELATED APPLICATIONS

This application is a continuation of U.S. application Ser. No. 17/046,341, filed Oct. 9, 2020, which is a 371 National Phase Entry of International Patent Application No. PCT/US2019/026435, filed Apr. 9, 2019, which claims benefit under 35 U.S.C. § 119(e) of U.S. Provisional Application No. 62/654,879, filed Apr. 9, 2018, the contents of which are incorporated herein by reference in their entirety.

### GOVERNMENT SUPPORT

This invention was made with Government support under Grant No. R21AI117440 awarded by the National Institutes of Health. The Government has certain rights in the invention.

### FIELD OF THE INVENTION

The field of the invention relates to the treatment of an autoimmune disease.

### INCORPORATION BY REFERENCE

The contents of the XML file named “11624-003US2-ST26” which was created on Aug. 14, 2025, and is 100,561 bytes in size, are hereby incorporated by reference in their entirety.

### BACKGROUND

Multiple sclerosis (MS) is a chronic inflammatory autoimmune disease of the central nervous system (CNS) in which the insulating myelin sheet becomes damaged/destroyed. The principal responsible cells are mature cytokine-producing myelin-directed autoimmune CD4 cells (variously known as ex-Th17 cells, Th1/Th17 cells, or pathogenic Th17 cells) that infiltrate the CNS where they undergo further reciprocal amplifying and activating interactions with infiltrated monocytes and monocyte-derived cells that are directly responsible for neural damage and inflammation. MS affects previously healthy young adults (peak age 20-35 years) and to a lesser extent older children, 400,000 in the U.S. Females are more frequently affected than males (prevalence 3:1). The disease is devastating on multiple levels: mental stress due to a prognosis both negative and uncertain, physical suffering, restrictions of activity and loss of income, as well as economic costs to the family and community. Though current treatments for MS exist, there is no cure for MS. Thus, new therapeutics aimed at treating MS, included relapsing-remitting MS, are needed.

### SUMMARY

The compositions and methods described herein are related, in part, to the discovery that CXCR6, a chemokine receptor, is expressed on, and is a biomarker for, the CD4 effector cells that produce multiple inflammatory cytokines including IFNγ and GM-CSF; rapidly proliferate; and induce experimental autoimmune encephalomyelitis (EAE) in a mouse model (pathogenic T cells).

In one aspect, described herein is a method for treating an autoimmune disease, comprising administering to a subject having an autoimmune disease an agent that targets CXCR6; wherein targeting CXCR6 results in the depletion of a cell expressing CXCR6 or population thereof.

In another aspect, described herein is a method for treating an autoimmune disease, comprising administering to a subject having an autoimmune disease an agent that inhibits SerpinB1.

In another aspect, described herein is a method for selecting a population of Th17 cells or Th17-derived cells, the method comprising measuring the level of CXCR6 in a population of candidate cells, and selecting cells which exhibit expression of CXCR6.

In another aspect, described herein is a method of treating an autoimmune disease, the method comprises: receiving the results of an assay that indicate an increase in the levels of CXCR6 in a biological sample from a subject compared with an appropriate control; and administering to the subject an agent that inhibits the level or activity of SerpinB1.

In another aspect, described herein is a method of decreasing a population of T cells expressing CXCR6, the method comprising: administering an agent that decreases the levels or activity of SerpinB1 in leukocytes.

In one embodiment of any of the aspects, the cell population is depleted by at least 10%, at least 20%, at least 30%, at least 40%, at least 50%, at least 60%, at least 70%, at least 80%, at least 90% or more as compared to an appropriate control.

In another embodiment of any of the aspects, the cell population is a Th17 or Th17-derived cell population.

In another embodiment of any of the aspects, the agent that targets CXCR6 is linked to at least a second agent.

In another embodiment of any of the aspects, the autoimmune disease is selected from the list consisting of Rheumatoid arthritis, Crohn's disease, lupus, celiac disease, Sjogren's syndrome, polymyalgia rheumatic, multiple sclerosis, ankylosing spondylitis, type 1 diabetes, alopecia areata, vasculitis, autoimmune uveitis, juvenile idiopathic arthritis, and temporal arteritis.

In another embodiment of any of the aspects, the autoimmune disease is multiple sclerosis.

In another embodiment of any of the aspects, the subject is human.

In another embodiment of any of the aspects, the agent that targets CXCR6 is selected from the group consisting of a small molecule, an antibody, and a peptide.

In another embodiment of any of the aspects, the agent that inhibits SerpinB1 is selected from the group consisting of a small molecule, an antibody, a peptide, a genome editing system, an antisense oligonucleotide, and an RNAi.

In another embodiment of any of the aspects, the antibody is a depleting antibody.

In another embodiment of any of the aspects, the RNAi is a microRNA, an siRNA, or a shRNA.

In another embodiment of any of the aspects, the inhibiting of SerpinB1 is inhibiting the expression level and/or activity of SerpinB1.

In another embodiment of any of the aspects, the expression level and/or activity of SerpinB1 is inhibited by at least 50%, at least 60%, at least 70%, at least 80%, at least 90%, or more as compared to an appropriate control.

In another embodiment of any of the aspects, the level of CXCR6 is increased by at least 2-fold, at least 3-fold, at least 4-fold, at least 5-fold, at least 6-fold, at least 7-fold, at least 8-fold, at least 9-fold, at least 10-fold, or more as compared to a reference level.

In another embodiment of any of the aspects, the assay is flow cytometry, reverse transcription-polymerase chain reaction (RT-PCR), RNA sequencing, or immunohistochemistry.

In another embodiment of any of the aspects, the subject is suspected of having, or has an autoimmune disease.

In another embodiment of any of the aspects, the method further comprises, detecting the levels of SerpinB1 expressed by Th17 cells in a subject; and receiving the results of an assay that indicate an increase in SerpinB1 levels compared with an appropriate control.

In another embodiment of any of the aspects, the method further comprises, detecting the levels of one or more of: perforin-A, granzyme A (GzmA), GzmC, interleukin-17 (IL-17), IL-6, IL-21, IL-23, interleukin-23 receptor (IL-23R), IL-7Rα and IL-1R1, interferon gamma (IFNγ), RAR Related Orphan Receptor C (Rorc), and granulocyte-macrophage colony-stimulating factor (GM-CSF) in the subject.

In another embodiment of any of the aspects, the method further comprises, detecting leukocyte accumulation in the spinal cord.

In another embodiment of any of the aspects, prior to receiving the results of an assay the method comprises obtaining a biological sample from the subject.

In another embodiment of any of the aspects, the biological sample is synovial fluid, spinal fluid, tissue, or blood.

In another embodiment of any of the aspects, the said decreasing the levels or activity of SerpinB1 in leukocytes comprises administering an inhibitor of SerpinB1.

In another embodiment of any of the aspects, the T cell population is depleted by at least 10%, at least 20%, at least 30%, at least 40%, at least 50%, at least 60%, at least 70%, at least 80%, at least 90% or more as compared to an appropriate control.

In another embodiment of any of the aspects, the T cell population is a Th17 or Th17-derived cell population.

In another embodiment of any of the aspects, said decreasing levels or activity of SerpinB1 is in a subject in need of treatment for an autoimmune disease.

In another embodiment of any of the aspects, the agent is selected from the group consisting of: a small molecule, an antibody, a peptide, a genome editing system, an antisense oligonucleotide, and an RNAi.

In another embodiment of any of the aspects, the level and/or activity of SerpinB1 is inhibited by at least 50%, at least 60%, at least 70%, at least 80%, at least 90%, or more as compared to an appropriate control.

In another embodiment of any of the aspects, the administering inhibits inflammation.

In another embodiment of any of the aspects, the administering inhibits leukocyte accumulation in the spinal cord.

### BRIEF DESCRIPTION OF THE DRAWINGS

FIGS. 1A and 1B present data that show Serpinb1 (Sb1), a protease inhibitor, is a signature gene of Th17 cells. FIG. 1A shows Western blot showing SerpinB1 levels in Th17 cells. FIG. 1B shows mRNA levels of indicated gene in effector CD4 cells in EAE (Day 10) and naïve (Day 0) mice.

FIGS. 2A and 2B present data that show EAE in mice with global deletion of Serpinb1 (Sb1^{-/-}). FIG. 2A shows the characteristics of disease in indicated mouse. FIG. 2B shows characterization of spinal cord cells in indicated mouse. Sb1 is essential for pathogenicity of EAE. Sb1 is essential for CNS infiltration of CD4 cells

FIGS. 3A and 3B present data that show EAE in two models of Sb1 deletion in T cells. FIG. 3A shows adoptive transfer of CD4 T cells recovered from immunized wild-type or sb1^{-/-} mice into naïve WT mice (top) or from WT mice into naïve WT or sb1^{-/-} mice. FIG. 3B shows transfer of naive CD4 T cells from naive wild-type or sb1^{-/-} mice into Rag^{-/} mice that were then immunized to induce EAE. Disease amelioration only requires serpinb1 deletion in T cells or only in CD4 T cells.

FIGS. 4A and 4B present data that show EAE in Sb1^{-/-}:WT mixed chimeric mice. FIG. 4A shows the clinical score depicting disease severity. FIG. 4B shows CD4 cell ratio at indicated time points, and in various organs. Sb1^{-/-} CD4 cells are preferentially depleted in the spinal cord.

FIG. 5A-5I present data that show CD4 cell differentiation to Th17 cells in peripheral lymphoid organs. FIG. 5A shows the quantification of immune cells. FIG. 5B shows T-effectors. FIG. 5C shows T regulatory cells (Tregs). FIG. 5D shows chemokine receptors. FIG. 5E shows antigen recall and IL-17 production. FIG. 5F shows the IL-1 receptor. FIG. 5G shows metabolic enzymes. FIG. 5H shows integrins, and FIG. 5I shows cytokines in a wild-type (black bar) or Sb1^{-/-} (gray bar) mouse. Serpinb1 is not required to generate antigen-specific IL-17⁺ CD4 effector cells.

FIG. 6A-6C present data that show IFNγ+ and GM-CSF+ effector CD4 cells in WT and Sb1^{-/-} mice. FIG. 6A shows the quantification of various cytokine-producing CD4 effector cells following PMA plus ionomycin. FIG. 6B shows the quantification of antigen recall. FIG. 6C shows the mRNA levels in lymph node effector CD4 cells quantified by real time PCR. Gray symbols indicate Sb1^{-/-}; black indicate WT^{.-}IFNγ⁺ and GM-CSF⁺ effector CD4 cells are decreased in Sb1^{-/-} mice.

FIG. 7A-7E present data that identify genes differentially expressed in Sb1^{-/-} and WT CD4 effector cells. FIG. 7A shows expression level of 9649 genes determined by RNA sequencing of CD4 effector cells of indicated mice. FIG. 7B shows the 218 genes with expression decreased by a factor of 2 or more in Sb1^{-/-} compared with WT mice. FIG. 7C shows mRNA levels quantified by real time PCR. FIG. 7D shows CXCR6 expression in CD4 cells in indicated mouse. FIG. 7E shows CXCR6 expression in spinal cord infiltrated CD4 cells. Gray symbols indicate Sb1^{-/-}; black indicate WT. Genes underrepresented in CD4 effector cells of sb1^{-/-} mice encode IFNγ (Ifng) and GM-CSF (csf2) (as anticipated) and also cell surface CXCR6 (Cxcr6), the granule protease granzyme-C (Gzmc) and the pore-forming granule protein perforin (Pfr1).

FIG. 8A-8F present data that show the CXCR6-bearing subset of WT CD4 effector cells produces multiple cytokines and expresses Gzmc and Prf1 and highly express IL-1 and IL-23 receptors on the surface. FIG. 8A shows CXCR6 identifies the IL-17⁺, GM-CSF⁺ and IFNγ⁺ CD4 effector cells in EAE. FIG. 8B shows gene expression of indicated CD4 cells of WT mice in EAE. FIG. 8C shows activation markers and cytokine receptors expression on the surface of indicated CD4 cells in EAE. FIG. 8D-8F shows Granzyme C and perforin are highly expressed in CXCR6⁺CD4 cells, especially in the cells producing two or more of the cytokines IL-17 and/or IFNγ and/or GM-CSF. Immunized wild-type mice were used to characterize the properties of CXCR6⁺CD4 cells.

FIGS. 9A and 9B present data that show SerpinB1 inhibits Granzyme C. FIG. 9A shows gold staining of protein showing formation of an inactive covalent higher molecular weight complex on incubation of pure SerpinB1 with pure Granzyme C. FIG. 9B shows Western blot analysis of Granzyme C in the covalent complex with SerpinB1. Formation of a covalent complex with target proteases is the inhibitory mechanism unique to Serpins.

FIG. 10A-10F present data that show CXCR6 also marks “delayed hypersensitivity” CD4 cells that are generated in response to antigen, produce multiple cytokines, require serpinB1 for expansion and for induction of footpad swelling on challenge with antigen. FIG. 10A-10C shows Naïve WT ovalbumin (OVA)-sensitive (OT-II) cells were transferred into naïve WT mice, then immunized with OVA peptide. FIG. 10A shows CXCR6⁺OT-II cells quantified on the indicated days. FIG. 10B shows CXCR6⁺OT-II cells produce multiple cytokines as in the EAE system. FIG. 10C shows CXCR6⁺OT-II cells highly express granzyme C as in the EAE system. FIG. 10D-10E shows WT and sb1^{-/-} OT-II cells were transferred into naïve WT mice and then immunized with OVA peptide. FIG. 10D shows On day 10, total OT-II cells and CXCR6⁺OT-II cells were quantified. FIG. 10E shows that on day 7, indicated mice were challenged with OVA peptide in the footpad, and footpad swelling was quantified 24 hours later. FIG. 10F shows that WT and sb1^{-/-} mice were immunized with MOG peptide by the method that induces EAE. On day 6, the mice were challenged with MOG peptide in the footpad, and footpad swelling was measured at indicated times. FIG. 10D-F gray symbols indicate Sb1^{-/-}; black indicate WT.

FIG. 11A-11G present data that show markers of proliferation and markers of survival of CXCR6+ WT and Sb1^{-/-} CD4 effector cells in EAE. FIG. 11A shows Ki-67 labeling in indicated CD4 cell subsets. FIG. 11B shows quantification of BrdU in vivo labeling of the indicated CD4 cells. FIG. 11C shows dynamic analysis of BrdU incorporation in CXCR6⁺CD4 cells. Proliferation of CXCR6⁺CD4 cells is robust and is not different between WT and sb1^{-/-} cells. But the sb1^{-/-}CXCR6⁺CD4 cells have increased cell death. FIG. 11D shows Quantitation of annexin V staining of indicated CD4 cells. FIG. 11E shows quantification of indicated cells expressing active-Caspase 3. FIG. 11F-11G shows quantification of cells with damaged mitochondria in indicated mice. Sb1 is not required for proliferation of CXCR6⁺ CD4 effector cells. However, cell death of CXCR6⁺ CD4 effector cells is increased in sb1^{-/-} mice. Gray symbols indicate Sb1^{-/-}; black indicate WT.

FIG. 12A-12D present data that show anti-CXCR6 antibody treatment prevents EAE. WT mice were induced for EAE, and then treated with isotype control antibody (8 mice) or anti-mouse CXCR6 antibody (7 mice) (300 μg/mouse/injection) at days 5, 7, 9 and 12. FIG. 12A shows mean clinical score. FIG. 12B shows mean body weights. FIG. 12C shows frequency of diseased mice. FIG. 12D shows infiltrated lymphocytes and myeloid cells in the spinal cord on day 27.

FIGS. 13A and 13B present data that show treatment with anti-CXCR6 antibody is effective as a treatment for EAE. FIG. 13A shows WT mice were induced for EAE. Treatment with isotype control antibody (400 μg/treatment) (11 mice) or anti-CXCR6 antibody (8 mice) was initiated for individual mice on the day that disease was first detected (days 11-15; initial scores 1-3). Subsequent treatments were administered 2 and 4 days later (arrows). (FIG. 13A, top panel) Mean clinical score. (FIG. 13A, bottom panel) Body weight. FIG. 13B shows six WT mice were induced for EAE, and three mice each were treated on day 10 with 400 μg isotype control or anti-CXCR6 antibody; the mice were sacrificed on day 11. (FIG. 13B, top-panel) Representative flow cytometry measuring cytokines IL-17 and GM-CSF production by lymph node CD4 cells. (FIG. 13B, bottom panel). Cumulative results for production of these cytokines. The decrease of cytokine-producing cells indicates that the anti-CXCR6 antibody treatment depletes the cytokine-producing CXCR6⁺ pathogenic CD4 cells.

FIG. 14A-14C present data that show human CXCR6+ CD4 cells are present in inflammatory synovial fluids of patients with rheumatoid arthritis and these cells produce multiple cytokines as in the murine EAE system. FIG. 14A shows background information on pathogenic CD4 cells in autoimmune disorders. FIG. 14B-14C shows analysis of synovial fluid cells of two patients with rheumatoid arthritis including frequency of CXCR6+ CD4 cells and their production of IL-17, IFNγ and GM-CSF.

FIG. 15 presents a schematic that shows the generation of CXCR6+ cells based on the EAE data. The regulatory step is indicated in which sb1 prevents cell death of robustly proliferating CXCR6+ cells by inhibiting a protease, which may be the human equivalent of murine granzyme C, and thereby determines the size of the resulting population of pathogenic CXCR6+ CD4 cells.

FIG. 16A-16E shows Serpinb1a (Sb1) is highly expressed in TH cells in EAE. FIG. 16A shows SerpinB1 expression in wt T cell subsets differentiated in vitro and analysed by Western blot. Data are representative of five experiments. FIG. 16B shows Sb1, Rorc and Il17a are expressed in effector CD4 cells at onset of EAE. Transcripts were quantified by qRT-PCR for CD44+ (effector) CD4 cells isolated from lymph nodes of naïve mice (Day 0) and MOG/CFA induced EAE mice at disease onset (Day 10). Data are representative of two experiments with pooled cells from nine naïve and nine EAE mice. FIG. 16C-D shows RNA Seq analysis. Mixed chimeric mice (CD45.1 wt/CD45.2 Il23rΔCD4) were immunized with MOG/CFA to induce EAE. On day 13, effector (CD44+) CD4 cells were sorted from draining lymph nodes. FIG. 16C shows gene expression in wt and Il23rΔCD4 effector (CD44+) CD4 cells. Data are mean of five replicates with 3-4 chimeric mice per replicate. FIG. 16D shows top hits with identities. FIG. 16E shows IL-23 treatment maintains expression of Sb1, Rorc and Il17a in Th17 cells. In vitro differentiated TH17 cells were maintained in IL-2 for 2 days and re-stimulated with anti-CD3/CD28 and the indicated cytokine for 24 h and then analyzed by qRT-PCR Data are representative of three experiments.

FIG. 17A-17G shows CD4 cell autonomous deficiency of sb1 ameliorates EAE. (A-C) Wt and sb1^{-/-} mice were immunized with MOG/CFA to induce EAE. FIG. 17A shows mean clinical score (left) and body weight (right) of wt (n=13) and sb1^{-/-} (n=14) mice. Experiment was repeated more than 5 times with the same pattern. FIG. 17B shows spinal cord infiltrates on day 10 analysed by flow cytometry. n=4-5 mice each genotype representative of five experiments. FIG. 17C shows relative gene expression of spinal infiltrates analyzed by qRT-PCR Data represent mean of four biological replicates, each with pooled cells from 2-3 mice per genotype. FIG. 17D shows adoptive transfer EAE. Wt or sb1^{-/-} T cells from MOG-immunized mice were expanded ex vivo and transferred to naive wt or sb1^{-/-} recipients. Mean clinical scores for 6 mice each genotype. FIG. 17E shows naive CD4 cell transfer EAE. Wt or sb1^{-/-} naïve CD4 cells were transferred to Rag1/− mice, which were then MOG-immunized to induce EAE. Mean clinical scores for 6 mice each genotype. FIG. 17F shows DTH response of wt and sb1^{-/-} mice to challenge in the footpad with MOG or vehicle on day 6 post MOG immunization. FIG. 17G shows the ratio of sb1^{-/-} to wt CD4 cells in active EAE in chimeric mice. Symbols indicate individual mice. Data are representative of two (FIGS. 17D, 17F, and 17G) or three (FIG. 17E) experiments. Error bars indicate ±SEM. *p<0.05; **p<0.01 by Student's t-test (FIGS. 17C and 2F); ***p<0.001 by one-way ANOVA (FIG. 17G).

FIG. 18A-18F shows decreased frequency of IFNγ+ and GM-CSF+ CD4 cells in lymph node of sb1−/−mice provided the key to signature genes of pathogenic TH cells. FIG. 18A-3B shows decreased frequency of sb1−/− IFNγ+− and GM-CSF+ CD4 cells at onset of EAE. FIG. 18A shows mRNA. Relative gene expression of effector (CD44+) CD4 cells determined by qRT-PCR Depicted data are mean±SEM for pooled cells of 3-5 mice per genotype in three experiments. FIG. 18B shows Cytokine-producing CD4 cells analyzed by flow cytometry after ex vivo stimulation with P+I. (Left) Representative contour plots of LN CD4 cells. (Right) Cumulative frequencies for LN and spinal cord CD4 cells. Data for 5 mice per genotype are representative of five experiments. FIG. 18C shows RNA Seq analysis. RNA of wt and sb1−/− LN effector (CD44+) CD4 cells harvested at disease onset and incubated with P+I. Depicted (left and middle) are the 9,650 genes with expression levels (FPKM)>1.0. Area above the dashed lines in the middle panel depicts the 258 genes with sb1−/− expression relative to wt decreased by >2.0-fold. Identities are indicated for the verified genes (right panel). FIG. 18D shows verification by qRT-PCR that Prf1, Gzma, Gzmc, Ifng and Csf2 expression are decreased in sb1−/− LN effector CD4 cells. Depicted data are representative of two cell isolates analyzed after P+I stimulation. FIG. 18E-18F shows CXCR6 expression on CD4 cells of MOG-immunized wt and sb1−/−mice. Depicted are (left) representative plots and (middle) mean frequencies and (right) absolute cell numbers for (FIG. 18E) lymph nodes on day 0 (naïve mice), day 7 (pre-disease) and day 10 (disease onset) and (FIG. 18F) spinal cord on day 14 (peak disease). Data for 3-6 mice per time point per experiment are representative of two experiments. Symbols in (FIG. 18F) indicate individual mice; horizontal lines indicate mean. Error bars represent ±SEM. *p<0.05; **p<0.01, ***p<0.001 by Student's t-test.

FIG. 19A-19F shows pathogenic TH cells are marked by CXCR6 and produce multiple cytokines and express GzmC and perforin. MOG-immunized wt mice were sacrificed at onset of EAE, and lymph node cells were analyzed. FIG. 19A-4B show CXCR6 expression on cytokine-producing CD4 cells. FIG. 19A shows representative dot plots. FIG. 19B shows cumulative frequencies from three experiments. Symbols indicate individual mice; horizontal bars indicate mean. FIG. 19C shows GzmC and GzmB in naïve, CXCR6neg− and CXCR6+-effector CD4 cells analysed by flow cytometry. Depicted are representative data of four experiments. FIG. 19D shows Perforin expression in cytokine-producing cells detected by intracellular staining and flow cytometry. Symbols indicate individual mice; horizontal bars indicate mean. Data are representative of two experiments. FIG. 19E shows relative gene expression of CCR6+CXCR6neg− and CXCR6+-effector CD4 cells analyzed by RT-qPCR, Data are representative of two experiments. FIG. 19F shows histograms of IL-7Ra, IL-23R, IL-1R1 and CD69 on CXCR6neg and CXCR6+ CD4 effector cells. Depicted data are for pooled cells of 5-9 mice wt mice per experiment and are representative of two experiments. Error bars represent ±SEM.

FIG. 20A-20E shows anti-CXCR6 treatment prevents EAE and reverses established disease. FIG. 20A-5B shows disease prevention protocol. Wt mice were immunized with MOG and treated with anti-mouse CXCR6 mAb or isotype control (300 jig i.p.) on days 5 (pre-disease), 7, 9 and 12 (arrows). FIG. 20A shows clinical score (mean f SEM) and FIG. 20B shows disease frequency (n=8 per group). One diseased mouse in the isotype-treated group recovered spontaneously on day 22. FIG. 20C-20E shows the therapeutic protocol. Wt mice were MOG-immunized, and when disease was first detected (clinical score 1-3), the mice were randomly assigned to receive either anti-CXCR6 antibody (n=8) or isotype control (n=11) (400 jig i.p.) on that day and 2 and 4 days later (arrows). FIG. 20C shows the clinical score and FIG. 20D shows body weight. Data are mean±SEM. FIG. 20E shows the histology. Representative spinal cord sections on day 11 of therapeutic treatment stained with hematoxylin and eosin. Histopathology scores (inflammation, degeneration) are shown on the right. Videos 1-5 (EXAMPLE 3) show the behavior of the mAb-treated and isotype-control mice.

FIG. 21A-21E shows CXCR6 expression in OT-II cells. (A-C) OT-II cell transfer studies. Naïve OT-II cells (CD45.2) were transferred into naïve congenic CD45.1 mice, followed by OVA immunization. FIG. 21A shows CXCR6+ OT-II cells in LN analyzed by flow cytometry on days 4 and 12. Left: Representative contour plots; Right: Cell frequencies. FIG. 21B shows IL-17 and GM-CSF expression in CXCR6neg and CXCR6+OT-II cells on day 12. FIG. 21C shows histogram of GzmC expression in LN CXCR6neg and CXCR6+ OT-II cells on day 12. FIG. 21D-6E shows sb1−/− OT-II transfer studies. Naïve wt OT-II and sb1−/−OT-II cells were separately transferred as in panel A, and the mice were immunized with OVA. FIG. 21D shows CXCR6-expressing wt and sb1−/− OT-II cells in LN on day 10 analysed by flow cytometry. (Left) Representative contour plots; (right) mean cell frequencies. FIG. 21E shows OVA-induced DTH. Footpad swelling in mice transferred with wt or sb1−/− OT-II cells, immunized with OVA and challenged in the footpad with OVA peptide. Footpad swelling was measured 24 hr after challenge. Symbols represent individual mice. Data are representative of (FIG. 21A) three and (FIG. 21B-21E) two experiments. *p<0.05 by Student's t-test.

FIG. 22A-22D shows CXCR6 expression on synovial fluid (SF) CD4 cells of patients with inflammatory arthritis. FIG. 22A shows representative histograms for CXCR6+CD4 cells in SF of two patients. FIG. 22B shows cumulative frequency of CXCR6+CD4 cells in SF of nine patients and PBMC of two healthy donors. FIG. 22C shows representative FACS plots showing co-expression of cytokines and CXCR6 on synovial fluid CD4 cells of inflammatory arthritis patients. Cells were incubated with P+I for 4 hr. FIG. 22D shows Pearson's correlation coefficients for frequency of CXCR6+ cells and different cytokine-expressing cells. Because the cytokine-producing cell incubation with P+I cause CXCR6 to be downregulated, the results of separate assays were used to determine correlation coefficients. Symbols indicate individual patients. *p<0.05, **p<0.01, **p<0.001 by Student's t-test.

FIG. 23A-23G shows CXCR6+TH cells of Sb1−/− mice are subject to enhanced cell death during robust proliferation. Wt and sb1−/− mice were immunized with MOG/CFA to induce EAE. FIG. 23A shows the frequency of BrdU+ CD4 cells quantified by flow cytometry after in vivo labeling for 6 hr or 2 hr or 1 hr. Data are representative of 2-3 experiments. Symbols represent individual mice; horizontal lines represent mean. FIG. 23B shows Ki-67 mAb staining of freshly isolated LN CD4 cells at disease onset. Depicted histograms are representative of 7 mice per genotype in two experiments. FIG. 23C shows active Caspase-3 staining of freshly isolated LN CD4 cells at disease onset. FIG. 23D shows activated caspase-3 of cytokine-producing cells. LN cells were stimulated with P+I for 2.5 hr and stained for cytokines and activated caspase-3. Depicted data are representative of 2-3 experiments. FIG. 23E shows (Δψm) measured by retention of the mitochondrial dye DiOC6. Shown are representative histograms. FIG. 23F shows (Δψm) measured with the mitochondrial dye JC-1. Cells with intact mitochondria retain JC-1 and emit red fluorescence; cells with disrupted mitochondria emit green fluorescence. Data in (FIGS. 23E and 23F) are each representative of two experiments with 5 mice per genotype. FIG. 23G shows recombinant human SerpinB1 (rhSB1) forms an inhibitory complex with rGzmC. Western blot stained with rabbit anti-GzmC. Arrows indicate GzmC (Glu193Gly) at 26 kD and the covalent SB1-GzmC complex (cpx) at 66 kD. SB1, detected in a parallel protein-stained gel, migrates at 42 kD. Data are representative of three experiments. *p<0.05, **p<0.01 by Student's t-test.

FIG. 24A-24J shows Serpinb1-deficient CD4 cells are not defective in the priming phase of EAE. FIG. 24A-24E, 24G-24I show Wt and sb1−/− mice were immunized with MOG, and draining lymph node (LN) cells were studied at onset of EAE. FIG. 24A shows immune cell counts. FIG. 24B shows T effector cell frequency. FIG. 24C shows Tregs (CD4+CD25hiFoxP3+) frequency. FIG. 24D shows CCR2+ and CCR6+ CD4 cell frequency. Data were determined by flow cytometry and are means for 9 mice each genotype. FIG. 24E shows IL-17 production in antigen-recall reaction. FIG. 24F shows responsiveness to IL-23 in antigen recall. Splenocytes (FIG. 24E) or LN cells (FIG. 24F) harvested at disease onset were cultured with or without MOG in the presence or absence of IL-23 for 48 hr and (FIG. 24E) IL-17 in supernatants was quantified by ELISA or (FIG. 24F) BFA was added during the last 8 h, and IL-17+ cells were quantified by intracellular flow cytometry. Depicted data are means for (FIG. 24E) 8 and (FIG. 24F) 5 mice per genotype representative of 2-3 experiments. FIG. 24G shows IL-1 receptor upregulation. Frequency of IL-1R+CD4 cells in LN on days 0, 6 (pre-disease) and day 10 (onset of EAE) post-immunization of Rag1/− mice transferred with naïve wt and sb1−/− CD4 cells. Depicted data are means for 3-4 mice per genotype per time point. FIG. 24H shows relative expression of metabolic enzymes determined by qRT-PCR of effector (CD44+) CD4 cells. FIG. 24I shows Cell surface expression (mean fluorescence intensity; MFI) of integrin subunits of LN effector CD4 cells. Data are mean for 3-5 mice each genotype representative of three experiments. FIG. 24J shows relative expression of myeloid cell cytokines determined by qRT-PCR analysis of total LN immune cells. FIG. 24H, 24J show depicted data are means for pooled cells of 3-5 mice per genotype in three experiments. *p<0.05 by Student's t-test. Error bars represent SEM.

FIG. 25A-25B shows a decrease of cytokine-expressing sb1−/− CD4 cells in EAE (related to FIG. 18). FIG. 25A shows decreased number of IFNγ+ and GM-CSF+ CD4 cells at disease onset in (left) LN and (right) spinal cord. Shown are absolute cell numbers for the experiments of FIGS. 18B, 18C. FIG. 25B shows the frequency of cytokine-producing wt and sb1−/− CD4 cells in mixed chimeric mice at peak disease determined by flow cytometry after ex vivo stimulation with P+I. Lines connects wt (black circles) and sb1−/− (gray circles) cells from the same chimera. Experiments were repeated twice with the same pattern. *p<0.05, **p<0.01, ***p<0.001 by Student's paired t-test.

FIG. 26A-26B shows the effects of anti-CXCR6 treatment on disease (related to FIG. 20C-20E). FIG. 26A shows a feasibility study: Testing whether anti-CXCR6 treatment alters the frequency of cytokine+ CD4 cells in lymph nodes. MOG-immunized wt mice received a single dose (400 μg i.p.) of anti-CXCR6 mAb or isotype control at disease onset and were sacrificed 24 hr later. LN cells were stimulated with P+I and analyzed for cytokine expression by flow cytometry. Symbols represent individual mice. Results are representative of two experiments. FIG. 26B shows a prevention protocol: Myeloid cells and lymphocytes harvested from spinal cord of isotype-treated and anti-CXCR6 treated wt mice at day 30 (termination) of the FIG. 20A, 20B study. (Left) Representative flow cytometry. (Right) Mean count of myeloid cells and lymphocytes. *p<0.05, **p<0.01 by Student's t-test. Error bars represent ±SEM.

FIG. 27 shows Table 1 of synovial fluid samples of patients with inflammatory arthritis.

FIG. 28 shows Table 2 or primer sequences. FIG. 28 discloses SEQ ID NOs: 9-76, respectively, in order of appearance from top to bottom and left to right.

### DETAILED DESCRIPTION

The invention described herein relates to, in part, the discovery that the cell surface protein CXCR6 is a trackable marker that identifies IFNγ- and GM-CSF-producing and highly proliferating pathogenic CD4 cells, and renders the cells amenable to direct study and manipulation is innovative. It is contemplated herein that the administration of an agent that targets CXCR6 (e.g., an anti-CXCR6 depleting antibody) to a subject having an autoimmune disease will effectively kill CXCR6-expressing cells, and lessen the severity of, or treat, an autoimmune disease (e.g., multiple sclerosis).

Additionally, presented herein is data that show that SerpinB1 regulates expansion of pathogenic CD4 cells in the murine multiple sclerosis model (EAE). It is specifically contemplated herein that the administration of an agent that inhibits SerpinB1 to a subject having an immune disease will target CXCR6⁺ pathogenic CD4 cells to prevent the expansion of these cells, and treat the autoimmune disease. Further, work presented herein indicates that pathogenic CD4 cells in EAE contain cytotoxic granules.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with an autoimmune disease or disorder, e.g. multiple sclerosis. The term “treating” includes reducing or alleviating at least one adverse effect or symptom of an autoimmune disease or disorder, e.g., multiple sclerosis (e.g., muscle tremors). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a therapeutic (e.g., an agent that targets CXCR6 or inhibits SerpinB1) or pharmaceutical composition as disclosed herein into a subject by a method or route which results in at least partial delivery of the agent to the subject. Pharmaceutical compositions comprising agents as disclosed herein can be administered by any appropriate route which results in an effective treatment in the subject.

As used herein, the term “contacting” when used in reference to a cell or organ, encompasses both introducing or administering an agent, surface, hormone, etc. to the cell, tissue, or organ in a manner that permits physical contact of the cell with the agent, surface, hormone etc., and introducing an element, such as a genetic construct or vector, that permits the expression of an agent, such as a miRNA, polypeptide, or other expression product in the cell. It should be understood that a cell genetically modified to express an agent, is “contacted” with the agent, as are the cell's progeny that express the agent.

As used herein, a “subject” means a human or animal. Usually the animal is a vertebrate such as a primate, rodent, domestic animal or game animal. Primates include, for example, chimpanzees, cynomolgous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., autoimmune disease. A subject can be male or female.

A subject can be one who has been previously diagnosed with or identified as suffering from or having a disease or disorder in need of treatment (e.g., an autoimmune disease) or one or more complications related to such a disease or disorder, and optionally, have already undergone treatment for the disease or disorder or the one or more complications related to the disease or disorder. Alternatively, a subject can also be one who has not been previously diagnosed as having such disease or disorder (e.g., autoimmune disease) or related complications. For example, a subject can be one who exhibits one or more risk factors for the disease or disorder or one or more complications related to the disease or disorder or a subject who does not exhibit risk factors.

As used herein, “targets”, or “targeting” refers to an agent that will localize to and bind to a given target (e.g., CXCR6). An agent can localize to or bind to the full length of the target (e.g., the nucleotide sequence of SEQ ID NO: 1, or the amino acid sequence of SEQ ID NO: 3), or a fragment thereof that is sufficient for the agent to localize to or bind to. An agent can target CXCR6, e.g., directly, or indirectly. Wherein “targeting” results in the agent binding the given target (e.g., CXCR6), the binding can be irreversible or reversible.

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of the polypeptide or the polynucleotide. Agents that inhibit SerpinB1, e.g., inhibit expression, e.g., translation, post-translational processing, stability, degradation, or nuclear or cytoplasmic localization of a polypeptide, or transcription, post transcriptional processing, stability or degradation of a polynucleotide or bind to, partially or totally block stimulation, DNA binding, transcription factor activity or enzymatic activity, decrease, prevent, delay activation, inactivate, desensitize, or down regulate the activity of a polypeptide or polynucleotide. An agent can act directly or indirectly.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

Methods and compositions described herein require that the CXCR6 is targeted. As used herein, “C-X-C motif chemokine receptor 6 (CXCR6)” refers to a receptor on a subset of CD4 cells. Sequences for CXCR6, also known as BONZO, CD186, STRL33, and TYMSTR, are known for a number of species, e.g., human CXCR6 (NCBI Gene ID: 10663) polypeptide (e.g., NCBI Ref Seq NP_006555.1) and mRNA (e.g., NCBI Ref Seq NM_006564.1). CXCR6 can refer to human CXCR6, including naturally occurring variants, molecules, and alleles thereof. CXCR6 refers to the mammalian CXCR6 of, e.g., mouse, rat, rabbit, dog, cat, cow, horse, pig, and the like. The human nucleic sequence of SEQ ID NO:1 comprises the nucleic sequence which encodes CXCR6. The human polypeptide sequence of SEQ ID NO: 3 comprises the polypeptide sequence of CXCR6.

Methods and compositions described herein require that the levels and/or activity of SerpinB1 are inhibited. As used herein, “Serpin Family B Member 1 (SerpinB1)” or “leukocyte elastase inhibitor” refers to a protein known to inhibit e.g., neutrophil-derived proteinases, neutrophil elastase, cathepsin G, granzyme H, and proteinase-3. SerpinB1 has a role in protecting tissues from damage at inflammatory sites. SerpinB1 functions to promote expansion of CXCR6′ pathogenic CD4 cells. SerpinB1 sequences are known for a number of species, e.g., human SerpinB1 (NCBI Gene ID: 1992) polypeptide (e.g., NCBI Ref Seq NP_109591.1) and mRNA (e.g., NCBI Ref Seq NM_030666.3). SerpinB1 can refer to human SerpinB1, including naturally occurring variants, molecules, and alleles thereof. SerpinB1 refers to the mammalian SerpinB1 of, e.g., mouse, rat, rabbit, dog, cat, cow, horse, pig, and the like. The nucleic sequence of SEQ ID NO:2 comprises the nucleic sequence which encodes SerpinB1. The human polypeptide sequence of SEQ ID NO: 4 comprises the polypeptide sequence of SerpinB1.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to an appropriate control.

The terms “depletion”, “depleted,” or deplete” are used interchangeable herein as another term for decrease. With regard to the methods described herein, deplete can mean a decrease in the number of cells in a population. For example, deplete can mean that the number of cells expressing a specific marker (e.g. CXCR6) are reduced; no longer viable; or expanding in number. Depletion can occur physically or immunologically.

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

As used herein, the term “modulates” refers to an effect including increasing or decreasing a given parameter as those terms are defined herein.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with an autoimmune disease, or a biological sample that has not been contacted with an agent disclosed herein).

As used herein, an “appropriate control” refers to an untreated, otherwise identical cell or population (e.g., a patient who was not administered an agent described herein, or was administered by only a subset of agents described herein, as compared to a non-control cell).

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### Treating an Autoimmune Disease

In one aspect of any of the embodiments, described herein is a method for treating an autoimmune disease comprising administering to a subject having an autoimmune disease an agent that targets CXCR6.

In another aspect of any of the embodiments, described herein provides a method for treating an autoimmune disease comprising administering to a subject having an autoimmune disease an agent that inhibits SerpinB1.

In some embodiments of any of the aspects, the autoimmune disease is selected from the list consisting of Rheumatoid arthritis, Crohn's disease, lupus, celiac disease, Sjogren's syndrome, polymyalgia rheumatic, multiple sclerosis, ankylosing spondylitis, type 1 diabetes, alopecia areata, vasculitis, autoimmune uveitis, juvenile idiopathic arthritis, and temporal arteritis.

In some embodiments of any of the aspects, the autoimmune disease is multiple sclerosis.

In some embodiments of any of the aspects, the administering inhibits inflammation. In some embodiments of any of the aspects, the administering inhibits leukocyte accumulation in the spinal cord.

As used herein, an “autoimmune disease” or “autoimmune disorder” is characterized by the inability of one's immune system to distinguish between a foreign cell and a healthy cell. This results in one's immune system targeting one's healthy cells for programmed cell death. Non-limiting examples of an autoimmune disease or disorder include inflammatory arthritis, type 1 diabetes mellitus, multiples sclerosis, psoriasis, inflammatory bowel diseases, SLE, and vasculitis, allergic inflammation, such as allergic asthma, atopic dermatitis, and contact hypersensitivity. Other examples of auto-immune-related disease or disorder, but should not be construed to be limited to, include rheumatoid arthritis, multiple sclerosis (MS), systemic lupus erythematosus, Graves' disease (overactive thyroid), Hashimoto's thyroiditis (underactive thyroid), celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis and fibromyalgia (FM).

Autoimmune diseases are typically characterized by inflammation. As used herein, the term “inflammation” or “inflamed” refers to activation or recruitment of the immune system or immune cells (e.g. T cells, B cells, macrophages). A tissue that has inflammation can become reddened, white, swollen, hot, painful, exhibits a loss of function, or can have a film or mucus. Methods of identifying inflammation are well known in the art. Inflammation typically occurs following injury or infection by a microorganism but can also be an aberrant or idiopathic inflammatory condition.

Examples of symptoms of an autoimmune disease include but are not limited to accumulation of leukocytes to the spinal cord, accumulation of leukocytes in synovial fluid, pain, difficulty walking or breathing, paralysis, gastrointestinal discomfort or diarrhea, or extreme fatigue. A skilled practitioner or physician will be able to diagnose an autoimmune disease in a subject using standard techniques, e.g., blood tests, lumbar puncture, and non-invasive imaging (e.g., CT scan, or MRI).

Current methods of treating an autoimmune disease or disorder include medications, physical therapy, surgery and/or exercise. Medications for an autoimmune disease can include but are not limited to mitoxantrone, interferon β1a therapy, peginterferon beta 1a, azathioprine, fingolimod, natalizumab, glatiramer, steroids (e.g. prednisolone, methylprednisolone, cortisone, hydrocortisone, budesonide,), analgesics and anti-inflammatory drugs (e.g. capsaicin, acetaminophen, ibuprofen, mesalamine), sulfasalazine, oxycodone, methotrexate, azathioprine, adalimumab, infliximab, mercaptopurine, hydroxychloroquine, antibiotics (e.g. clindamycin, metronidazole, aminosalicylic acid, penicillin), and vitamins (vitamin D). It is noted that the methods of treating an autoimmune disease as described herein can be carried out in addition to standard methods of treatment for an autoimmune disease.

The methods described herein can be applied to multiple sclerosis (MS) or arthritis, among others, as they are chronic and debilitating autoimmune diseases caused by inflammation and aberrant T cell activity. The clinical symptoms overlap in several aspects such as fatigue, problems moving, and weakness.

A mouse model of autoimmune encephalomyelitis (EAE), can model mammalian diseases such as chronic demyelinating disorders of the central nervous system driven by self-reactive T helper cells. The mouse model has been shown to be useful in identifying mechanisms of multiple sclerosis and other autoimmune diseases. Described herein, in part, is the discovery that the T helper cells that cause MS are identified as expressing CXCR6. Furthermore, these CXCR6 cell populations described herein are highly enriched in the synovial fluid (SF) of inflammatory arthritis patients. CXCR6 can broadly identify the pathogenic CD4 T cells in different autoimmune disorders by using OT-II CD4 T cell system and delayed-type hypersensitivity reaction, which is the prototype of CD4 T cell activation-mediated pathogenesis. Thus, in both mouse and human T helper cell (e.g. Th17 cells)-driven autoimmune disorders, CXCR6 identifies CD4 cells that produce multiple key pathogenic cytokines and are enriched in inflamed tissues.

In another aspect of any of the embodiments, described herein is a method of diagnosing an autoimmune disease. In another aspect of any of the embodiments, described herein is a method of treating an autoimmune disease, the method comprises receiving the results of an assay that indicate an increase in the levels of CXCR6 in a biological sample from a subject compared with an appropriate control; and administering to the subject an agent that inhibits the level or activity of SerpinB1.

In some embodiments of any of the aspects, the methods described herein further comprise detecting the levels of SerpinB1 expressed by Th17 cells in a subject; and receiving the results of an assay that indicate an increase in SerpinB1 levels compared with an appropriate control. In some embodiments of any of the aspects, the methods described herein further comprise detecting the levels of one or more of: perforin-A, granzyme A (GzmA), GzmC, interleukin-17 (IL-17), IL-6, IL-21, IL-23, interleukin-23 receptor (IL-23R), IL-7Rα and IL-1R1, interferon gamma (IFNγ), RAR Related Orphan Receptor C (Rorc), and granulocyte-macrophage colony-stimulating factor (GM-CSF) in the subject. In some embodiments of any of the aspects, the methods described herein further comprise detecting leukocyte accumulation in the spinal cord.

In some embodiments of any of the aspects, prior to receiving the results of an assay, the method further comprises, obtaining a biological sample from the subject.

The biological sample can be obtained by methods known in the art such as blood draw or surgical methods. In another embodiment of any of the aspects, the biological sample is synovial fluid, spinal fluid, a blood sample, buffy coat, serum, or tissue. In some embodiments, the tissue is from the gastrointestinal tract. In some embodiments, the tissue is a colonic tissue. Surgical removal of intestinal tissues are standard in the medical profession and methods are known in the art. For example, a colectomy, is a procedure in which part of the colon or a tissue sample from the colon is removed. This is typical in identifying autoimmune diseases such as Crohn's disease or forms of ulcerative colitis.

Methods of measuring any cell marker (e.g. CXCR6 or SerpinB1) are known in the art and can be carried out by a laboratory assay. In some embodiments, the assay is flow cytometry, reverse transcription-polymerase chain reaction (RT-PCR), RNA sequencing, or immunohistochemistry. The assay described herein can be performed any suitable container or apparatus available to one of skill in the art for cell culturing. For example, the assay can be performed in 24-, 96-, or 384-well plates. In one embodiment of any of the aspects, the assay is performed in a 384-well plate.

Cells for the aspects disclosed herein can be obtained from any source available to one of skill in the art. Additionally, cells can be of any origin and from any subject. Accordingly, in some embodiments, the cell is from a mammalian source. In some embodiments, of any of the aspects, the cells are leukocytes, lymphocytes, T cells, natural killer cells, macrophages, dendritic cells, B cells, lymphoid cells, endothelial cells, stem cells, or any cell type known in the art. In some embodiments, the T cells are T helper cells, Th17 cells, or Th17-derived cells. In some embodiments, the Th17 cells are positive for CXCR6 or SerpinB1. In some embodiments of any of the aspects, the cell is from a subject, e.g., a patient. In some embodiments of any of the aspects, the subject, is a patient in need of treatment for an autoimmune disease.

### Th17 and Th17-Derived Cells

In some embodiments of any of the aspects, the methods provided herein comprise modulating a cell population. In some embodiments of any of the aspects, the cell population is a Th17 or Th17-derived cell population.

As used herein, the term “T helper 17 cells” or “Th17 cells” refers to a type of pro-inflammatory T cell. T helper 17 cells have a myriad of cellular functions related to regulation of the adaptive immune response. For example, Th17 cells release pro-inflammatory cytokines, interferons, or granulocyte-macrophage colony-stimulating factor (GM-CSF) that recruit other inflammatory leukocytes (e.g. natural killer cells, macrophages, dendritic cells, etc.) to the site of action in the body.

As used herein, the term “cytokine” refers to a small protein (~5-20 kDa) that acts through a target cytokine receptor to modulate the immune response, cell growth, or other cellular functions. Examples of cytokines include but are not limited to interleukins (IL) such as IL-17, IL-25, IL-6, IL-21, IL-23, IL-1R1.

Aspects of the methods described herein target T helper 17 cells (Th17) or Th17-derived cells for programmed cell death in order to treat an autoimmune disease. Study of the MS-like murine disease, experimental autoimmune encephalomyelitis (EAE), has produced a breadth of understanding these cell types, starting with the differentiation of naive CD4 cells to IL-17-producing Th17 cells by a mechanism dependent on IL-1, IL-6 and TGFβ (reviewed in Weaver, 2006; Littman, 2010), and progressing to the subsequent conversion of Th17 cells to IFNγ- and GM-CSF-producing ex-Th17 cells (pathogenic Th17 cells) (Hirota, 2011). In addition to IL1/IL1R (Sutton, 2006), this latter conversion requires IL-23/IL-23R (McGeachy, 2009). The myeloencephalitic function of these T helper cells requires their production of GM-CSF (El-Behi, 2011; Codarri, 2011) and expression of the protease inhibitor serpinb1 (sb1) (Hou, 2016), however, the most downstream known requirement for CD4 cell pathogenicity.

### Depletion of CXCR6-Expressing Cells

In one embodiment of any of the aspects, administration of an agent that targets CXCR6 results in the depletion of a cell population expressing CXCR6. In one embodiment of any of the aspects, the agent is an anti-CXCR6 depleting antibody specific for human CXCR6. In another embodiment of any of the aspects, the agent is a humanized anti-CXCR6 depleting antibody.

As used herein, “depleting antibody” refers to an antibody that, upon binding its intended target (e.g., CXCR6) causes the cell expressing the intended target to undergo cell death (e.g., programmed cell death). The term “depleting antibody” also refers to an antibody that removes, reduces, or modulates a cell population. Immunological depletion can be carried out ex vivo, in vivo (the antibody is administered directly to the subject), or in vitro (the antibody treats a population of cells in culture). In ex vivo depletion, blood cells are removed from a subject, treated with the depleting antibody and blood cells can be transfused back into the patient. This is common, for example, to accomplish T cell depletion in graft vs. host disease (GVHD).

In one embodiment of any of the aspects, depleting results in programmed cell death in CXCR6-expressing cell. On skilled in the art will be able to assess whether a cell is or has undergone programmed cell death, e.g., using techniques described herein. In one embodiment of any of the aspects, depleting results in the inactivation or neutralization of a CXCR6-expressing cell (e.g., a cell that is no longer produces or received, e.g., cellular signals, or secrets, e.g., enzymes, or cytokines). One skilled in the art will be able to assess whether a cell is inactive or neutralized, e.g., by assessing the capacity of the cell to send or receive a cellular signal using, e.g., functional assays for a given signal transduction pathway, or secrete a cytokine, e.g., using ELISA.

In one embodiment of any of the aspects, the cell population expressing CXCR6 is a T helper 17 (Th17) cell population. In one embodiment of any of the aspects, the cell population expressing CXCR6 is a Th17-derived cell population. A Th17 cell is a subset of pro-inflammatory T helper cells. Th17 express, e.g., Interleukin-17. A Th17 cell population and/or Th17-derived cell population can be identified using techniques described herein below. In addition, a Th17 cell and a Th17-derived cell can be identified by expression of the transcription factor Rorc, or its gene product, e.g., RorγT. mRNA and/or protein levels of Rorc or RorγT can be measured as described herein.

In one embodiment of any of the aspects, the agent that targets CXCR6 depletes the CXCR6-expressing cell population by at least 10%, by at least 20%, by at least 30%, by at least 40%, by at least 50%, by at least 60%, by at least 70%, by at least 80%, by at least 90%, by at least 99%, or more as compared to an appropriate control. In one embodiment of any of the aspects, the agent results in the complete depletion of CXCR6-expressing cell population (e.g., 100% depletion of the cell population). As used herein, an appropriate control refers to the number of CXCR6-expressing cells prior to administration of the agent (e.g., anti-CXCR6 depleting antibody).

### Identifying a Population of Th17 or Th17-Derived Cells

One aspect of the invention described herein provides a method of identifying a population of Th17 or Th17-derived cells comprising measuring a level of CXCR6 in a population of candidate cells and selecting the cells that exhibits high expression of CXCR6.

In one embodiment of any of the aspects, a cell is a Th17 cell or Th17-derived cell if the level of CXCR6 is increased at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, or more as compared to the reference level, or at least 10%, at least 20%, at least 30%, at least 40%, at least 50%, at least 60%, at least 70%, at least 80%, at least 90%, or at least 99% or more as compared to the reference level. The reference level can be the level of CXCR6 in a cell that is not a Th17 or Th17-derived cell.

In another embodiment of any of the aspects, the candidate cells are contained in a biological sample. In one embodiment of any of the aspects, the candidate cells are in culture. In another embodiment of any of the aspects, the levels of CXCR6 are measured in vitro, or ex vivo. The levels of CXCR6 in the candidate cells can be measured using standard techniques, e.g., FACS analysis, or immunofluorescence. Protein and mRNA levels of CXCR6 can be assessed using western blotting or PCR-based assays, respectively. These methods are known by one of skill in the art.

In another embodiment of any of the aspects, the biological sample is taken from a subject that has previously been diagnosed with an autoimmune disease. In another embodiment of any of the aspects, the biological sample is taken from a subject that has not been diagnosed with an autoimmune disease.

### Agents

In one aspect of any of the embodiments, described herein is an agent that targets CXCR6 is administered to a subject having an autoimmune disease. In one embodiment of any of the aspects, an agent that targets CXCR6 is a small molecule, an antibody or antibody fragment, or a peptide.

In another aspect of any of the embodiments, an agent that inhibits SerpinB1 is administered to a subject having an autoimmune disease. In one embodiment of any of the aspects, the agent that inhibits SerpinB1 is a small molecule, an antibody or antibody fragment, a peptide, an antisense oligonucleotide, a genome editing system, or an RNAi.

In another aspect of any of the embodiments, described herein is a method of decreasing a population of T cells expressing CXCR6, the method comprises administering an agent that decreases the levels or activity of SerpinB1 in leukocytes.

An agent described herein targets SerpinB1 for its inhibition. An agent is considered effective for inhibiting SerpinB1 if, for example, upon administration, it inhibits the presence, amount, activity and/or level of SerpinB1 in the cell.

In one aspect of any of the embodiments, targeting CXCR6 results in the depletion of the CXCR6-expressing cell or population thereof.

In one embodiment of any of the aspects, inhibiting SerpinB1 inhibits the expansion of CXCR6⁺ pathogenic CD4 cells.

An agent can inhibit e.g., the transcription, or the translation of SerpinB1 in the cell. An agent can inhibit the activity or alter the activity (e.g., such that the activity no longer occurs, or occurs at a reduced rate) of SerpinB1 in the cell (e.g., SerpinB1's expression).

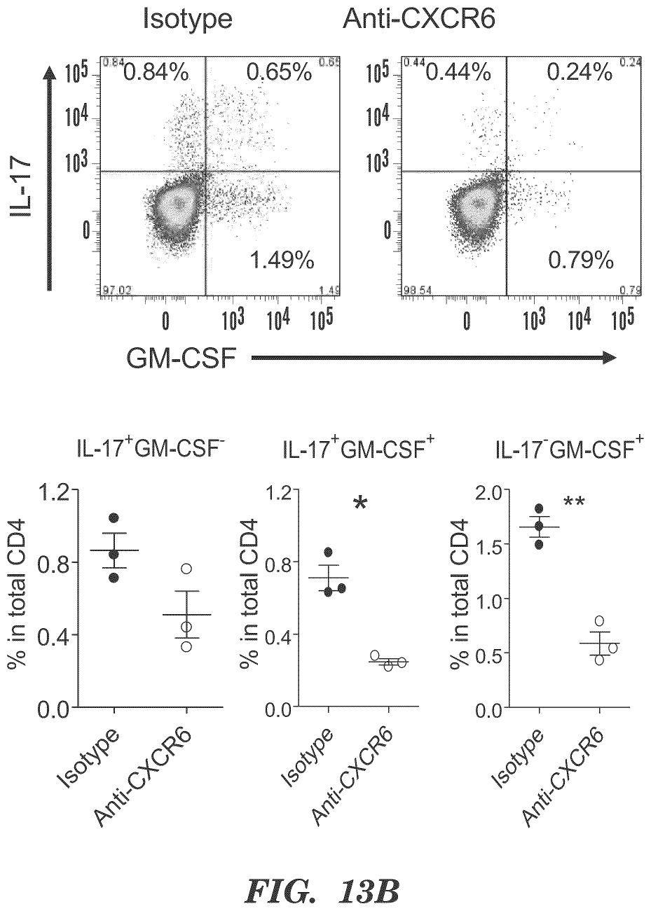

In one embodiment of any of the aspects, an agent that targets a cell expressing CXCR6 promotes programmed cell death, e.g., kills the cell. To determine if a cell has been targeted for programmed cell death, mRNA and protein levels of a given target (e.g., CXCR6) can be assessed using RT-PCR and western-blotting, respectively, and compared to an untreated, but identical, cell population. Biological assays that detect the activity of CXCR6 can be used to assess if a cell expressing CXCR6 has undergone programmed cell death. Alternatively, immunofluorescence detection using antibodies specific to CXCR6 in combination with cell death markers (e.g., Caspase) can be used to determine if cell death has occurred following administration of an agent that targets CXCR6. Additional methods for assessing cell death or cell depletion are described herein in the Examples and drawings, e.g., in FIG. 13B.

In one embodiment of any of the aspects, an agent that inhibits SerpinB1 promotes programmed cell death, e.g., kills the cell. To determine is an agent is effective at inhibiting SerpinB1, mRNA and protein levels of a given target (e.g., SerpinB1) can be assessed using RT-PCR and western-blotting, respectively. Biological assays that detect the activity of SerpinB1 (e.g., CXCR6⁺ pathogenic CD4 cells) can be used to assess if programmed cell death has occurred. Alternatively, immunofluorescence detection using antibodies specific to SerpinB1 in combination with cell death markers (e.g., Caspase) can be used to determine if cell death has occurred following administration of an agent.

In one embodiment of any of the aspects, an agent that inhibits the level and/or activity of SerpinB1 by at least 10%, by at least 20%, by at least 30%, by at least 40%, by at least 50%, by at least 60%, by at least 70%, by at least 80%, by at least 90%, by at least 100% or more as compared to an appropriate control. As used herein, an “appropriate control” refers to the level and/or activity of SerpinB1 prior to administration of the agent, or the level and/or activity of SerpinB1 in a population of cells that was not in contact with the agent. Inhibition of SerpinB1 will prevent the expansion of CXCR6⁺ pathogenic CD4 cells.

The agent may function directly in the form in which it is administered. Alternatively, the agent can be modified or utilized intracellularly to produce something which targets CXCR6 or inhibits SerpinB1, such as introduction of a nucleic acid sequence into the cell and its transcription resulting in the production of the nucleic acid and/or protein inhibitor of SerpinB1, or nucleic acid and/or protein that targets CXCR6 within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that targets CXCR6 or inhibits SerpinB1. Methods for screening small molecules are known in the art and can be used to identify a small molecule that is efficient at, for example, inducing cell death of pathogenic CD4 cells, given the desired target (e.g., CXCR6 or SerpinB1).

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

In some embodiments, the small molecule targets CXCR6 or SerpinB1.

In some embodiments, the agent is a polypeptide. As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues provided herein are preferred to be in the “L” isomeric form. However, residues in the “D” isomeric form may be substituted for any L-amino acid residue provided the desired properties of the polypeptide are retained.

In various embodiments of any of the aspects, the agent that targets CXCR6 or SerpinB1 is an antibody or antigen-binding fragment thereof, or an antibody reagent that is specific for CXCR6 or SerpinB1.

As used herein, the term “antibody” refers to a polypeptide that includes at least one immunoglobulin variable domain or immunoglobulin variable domain sequence and which specifically binds a given antigen. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment of any of the aspects, the agent that targets CXCR6 or SerpinB1 is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment of any of the aspects, the humanized antibody is a humanized monoclonal antibody. In another embodiment of any of the aspects, the humanized antibody is a humanized polyclonal antibody. In another embodiment of any of the aspects, the humanized antibody is for therapeutic use.

In another embodiment of any of the aspects, the anti-CXCR6 antibody is a depleting antibody.

In another embodiment of any of the aspects, the anti-CXCR6 antibody or antibody reagent is, at a minimum, sufficient to bind to CXCR6 but does not deplete the cell. In this embodiment, it is specifically contemplated that the anti-CXCR6 is an anti-CXCR6 targeting antibody. In this context, an “anti-CXCR6 targeting antibody” refers to an antibody that is used to target a CXCR6-expressing cell, or population thereof, and not result in the depleting of the cell. An “anti-CXCR6 targeting antibody” can comprise only a fragment of the full length antibody, e.g., only the Fab region, or only the CDR region, that is sufficient to bind CXCR6. An “anti-CXCR6 antibody targeting antibody” can be tethered or linked to other agents, moieties, toxins, or substances; these tethered or linked agents, moieties, toxins, or substances can be delivered to the CXCR6-expressing cell or population thereof, e.g., via binding of the anti-CXCR6 antibody targeting antibody to CXCR6.

In another embodiment of any of the aspects, the anti-CXCR6 targeting antibody is tethered or linked to an agent that inhibits SerpinB1.

In another embodiment of any of the aspects, the antibody or antibody reagent binds to an amino acid sequence that corresponds to the amino acid sequence encoding CXCR6 (SEQ ID NO: 3).

(SEQ ID NO: 3)
MAEHDYHEDY GFSSFNDSSQ EEHQDFLQFS KVFLPCMYLV
VFVCGLVGNS LVLVISIFYH KLQSLTDVFL VNLPLADLVF
VCTLPFWAYA GIHEWVFGQV MCKSLLGIYT INFYTSMLIL
TCITVDRFIV VVKATKAYNQ QAKRMTWGKV TSLLIWVISL
LVSLPQIIYG NVFNLDKLIC GYHDEAISTV VLATQMTLGF
FLPLLTMIVC YSVIIKTLLH AGGFQKHRSL KIIFLVMAVF
LLTQMPFNLM KFIRSTHWEY YAMTSFHYTI MVTEAIAYLR
ACLNPVLYAF VSLKFRKNFW KLVKDIGCLP YLGVSHQWKS
SEDNSKTFSA SHNVEATSMF QL

In another embodiment of any of the aspects, the anti-CXCR6 antibody or antibody reagent binds to an amino acid sequence that comprises the sequence of SEQ ID NO: 3; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 3. In another embodiment of any of the aspects, the anti-CXCR6 antibody or antibody reagent binds to an amino acid sequence that comprises the entire sequence of SEQ ID NO: 3. In another embodiment of any of the aspects, the antibody or antibody reagent binds to an amino acid sequence that comprises a fragment of the sequence of SEQ ID NO: 3, wherein the fragment is sufficient to bind its target, e.g., CXCR6, and result in the depletion of a CXCR6 expressing cell or population thereof.

In one embodiment of any of the aspects, the antibody or antibody reagent binds to an amino acid sequence that corresponds to the amino acid sequence encoding SerpinB1 (SEQ ID NO: 4).

(SEQ ID NO: 4)
MEQLSSANTR FALDLFLALS ENNPAGNIFI SPFSISSAMA
MVFLGTRGNT AAQLSKTFHF NTVEEVHSRF QSLNADINKR
GASYILKLAN RLYGEKTYNF LPEFLVSTQK TYGADLASVD
FQHASEDARK TINQWVKGQT EGKIPELLAS GMVDNMTKLV
LVNAIYFKGN WKDKFMKEAT TNAPFRLNKK DRKTVKMMYQ
KKKFAYGYIE DLKCRVLELP YQGEELSMVI LLPDDIEDES
TGLKKIEEQL TLEKLHEWTK PENLDFIEVN VSLPRFKLEE
SYTLNSDLAR LGVQDLFNSS KADLSGMSGA RDIFISKIVH
KSFVEVNEEG TEAAAATAGI ATFCMLMPEE NFTADHPFLF
FIRHNSSGSI LFLGRFSSP

In another embodiment of any of the aspects, the anti-SerpinB1 antibody or antibody reagent binds to an amino acid sequence that comprises the sequence of SEQ ID NO: 4; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 4. In one embodiment of any of the aspects, the anti-SerpinB1 antibody or antibody reagent binds to an amino acid sequence that comprises the entire sequence of SEQ ID NO: 4. In another embodiment of any of the aspects, the antibody or antibody reagent binds to an amino acid sequence that comprises a fragment of the sequence of SEQ ID NO: 4, wherein the fragment is sufficient to bind its target, e.g., SerpinB1, and result in the depletion of a CXCR6 expressing cell or population thereof.

In another embodiment of any of the aspects, the antibody or antibody reagent binds to an amino acid sequence that comprises the sequence of SEQ ID NO: 5, SEQ ID NO: 6, SEQ ID NO: 7, and/or SEQ ID NO: 8. In another embodiment of any of the aspects, the antibody or antibody reagent binds to an amino acid sequence that comprises a sequence with at least 80%, at least 85%, at least 90%, at least 95%, at least 96%, at least 97%, at least 98%, at least 99% or greater sequence identity to the sequence of any one of SEQ ID NOs: 1-8 or any of the sequences in the Table below. In some embodiments, the antibody or antibody reagent binds to an amino acid sequence that comprises the sequence of any one of SEQ ID NOs: 1-8 in any combination.

| Polypeptide name; NCBI Reference | |
|---|---|
| Sequence | SEQ ID NO: |
| CXCR6 (Homo sapiens); NCBI Reference | SEQ ID NO: 3 |
| Sequence: XP_011531593.1 | |
| SerpinB1 (Homo sapiens); NCBI Reference | SEQ ID NO: 4 |
| Sequence: NP_109591.1 | |
| SerpinB1 X1 isoform (Homo sapiens); | SEQ ID NO: 5 |
| NCBI Reference Sequence: | |
| XP_011512635.1 | |
| SerpinB1 X2 isoform (Homo sapiens); | SEQ ID NO: 6 |
| NCBI Reference Sequence: | |
| XP_011512637.1 | |
| CXCR6 (Mus musculus); NCBI Reference | SEQ ID NO: 7 |
| Sequence: NP_109637.3 | |
| SerpinB1 (Mus musculus); NCBI Reference | SEQ ID NO: 8 |
| Sequence: NP_079705.2 | |

In one embodiment of any of the aspects, an agent that targets CXCR6 is linked to at least a second agent. Delivery of an agent that targets CXCR6 that is linked to at least a second agent will direct the at least one second agent to a CXCR6-expressing cell. The second agent does not need to directly or indirectly target, effect, or interact with CXCR6. Alternatively, the second agent can directly or indirectly target, effect, or interact with CXCR6. In another embodiment of any of the aspects, the at least a second agent promotes programmed cell death of the CXCR6-expressing cell.

In one embodiment of any of the aspects, an antibody described herein is an anti-CXCR6 targeting antibody that is tethered to a nanoparticle. For example, an anti-CXCR6 targeting antibody bound to a nanoparticle can deliver the nanoparticle to a CXCR6-expressing cell, or population thereof, e.g., via binding of the anti-CXCR6 targeting antibody to CXCR6 on the cell surface. In another embodiment of any of the aspects, an antibody described herein is an anti-CXCR6 targeting antibody that is tethered to a small molecule. In another embodiment of any of the aspects, an antibody described herein is an anti-CXCR6 targeting antibody that is tethered to a moiety. In another embodiment of any of the aspects, an antibody described herein is an anti-CXCR6 targeting antibody that is tethered to a toxin. Exemplary toxins include, but are not limited to the anti-microtubule agent DM-1, a derivative of Maytansine, or monomethyl auristatin E (MMAE).

In one embodiment of any of the aspects, the agent that inhibits SerpinB1 is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., SerpinB1. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits SerpinB1 may comprise at least 5, at least 10, at least 15, at least 20, at least 25, at least 30, or more bases complementary to a portion of the coding sequence of the human SerpinB1 gene (e.g., SEQ ID NO: 2), respectively.

In one embodiment of any of the aspects, SerpinB1 is depleted from the cell's genome using any genome editing system including, but not limited to, zinc finger nucleases, TALENS, meganucleases, and CRISPR/Cas systems. In another embodiment of any of the aspects, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered in vivo, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment of any of the aspects, the agent inhibits SerpinB1 by RNA inhibition. Inhibitors of the expression of a given gene can be an inhibitory nucleic acid. In some embodiments of any of the aspects, the inhibitory nucleic acid is an inhibitory RNA (iRNA or RNAi). The RNAi can be single stranded or double stranded.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

The iRNA can be siRNA, shRNA, endogenous microRNA (miRNA), or artificial miRNA. In one embodiment of any of the aspects, an iRNA as described herein effects inhibition of the expression and/or activity of a target, e.g. SerpinB1. In some embodiments of any of the aspects, the agent is siRNA that inhibits SerpinB1. In some embodiments of any of the aspects, the agent is shRNA that inhibits SerpinB1.

One skilled in the art would be able to design siRNA, shRNA, or miRNA to target SerpinB1, e.g., using publically available design tools. siRNA, shRNA, or miRNA is commonly made using companies such as Dharmacon (Layfayette, CO) or Sigma Aldrich (St. Louis, MO).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

In one embodiment of any of the aspects, the agent is miRNA that inhibits SerpinB1. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of the target gene (e.g., SerpinB1), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effective target e.g., SerpinB1, for its downregulation, for example by transfecting the siRNA, shRNA, or miRNA into cells and detecting the levels of a gene (e.g., SerpinB1) found within the cell via western-blotting.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., an SerpinB1 inhibitor) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free cells. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

In one embodiment of any of the aspects, an agent can be bound to a polypeptide that binds to a CXCR6⁺ pathogenic CD4 cell, e.g., to target the agent to the CXCR6+ pathogenic CD4 cell. For example, a polypeptide that encodes the CXCR6 ligand, e.g., CXCL 16, or functional fragment thereof (e.g., a fragment that, at a minimum, binds CXCR6 or a portion thereof, but does not induce chemotaxis) can be bound to a small molecule to target the small molecule to a CXCR6-expressing cell. In one embodiment of any of the aspects, the agent is bound to a polypeptide encoding a non-activating CXCR6 ligand. In another embodiment of any of the aspects, a toxin can be bound to a polypeptide, or functional fragment thereof, that binds to a CXCR6⁺ pathogenic CD4 cell in order to target the toxin to the CXCR6-expressing cell. In it specifically contemplated herein that the binding of a peptide to CXCR6 does not induce activation of the receptor, e.g., it does not induce chemotaxis.

The agents provided herein can also include or be used in combination with those described in, for example, EP1003546B1; U.S. Pat. No. 7,931,900B2; U.S. Pat. No. 8,815,236B2; U.S. Pat. No. 6,329,499B1; U.S. Pat. No. 6,936,599B2; U.S. Pat. No. 6,495,579B1; U.S. Pat. No. 8,399,514B2; U.S. Pat. No. 7,320,999B2; EP0941089B1; EP0966300B1; U.S. Pat. No. 9,822,400B2; U.S. Pat. No. 9,546,212B2; US20170253651A1; which are incorporated herein by reference in their entirety.

### Pharmaceutical Compostions

In one embodiment of any of the aspects, the agent described herein is formulated with a pharmaceutical composition.

As used herein, the term “pharmaceutical composition” can include any material or substance that, when combined with an active ingredient (e.g. an antibody against CXCR6), allows the ingredient to retain biological activity and is non-reactive with the subject's immune system. Examples include, but are not limited to, any of the standard pharmaceutical carriers such as a phosphate buffered saline solution, emulsions such as oil/water emulsion, and various types of wetting agents. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. The term “pharmaceutically acceptable carrier” excludes tissue culture media. Each carrier must be “acceptable” in the sense of being compatible with the other ingredients of the formulation, for example the carrier does not decrease the impact of the agent on the treatment. In other words, a carrier is pharmaceutically inert. The terms “physiologically tolerable carriers” and “biocompatible delivery vehicles” are used interchangeably. Non-limiting examples of pharmaceutical carriers include particle or polymer-based vehicles such as nanoparticles, microparticles, polymer microspheres, or polymer-drug conjugates.

In some embodiments, the pharmaceutical composition is a liquid dosage form or solid dosage form. Liquid dosage forms for oral administration include, but are not limited to, pharmaceutically acceptable emulsions, microemulsions, solutions, suspensions, syrups and elixirs. In addition, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the agents described herein are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

Solid compositions of a similar type can also be employed as fillers in soft and hard-filled gelatin capsules using such excipients as lactose or milk sugar as well as high molecular weight polyethylene glycols, and the like. The solid dosage forms of tablets, dragées, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polyethylene glycols, and the like.

The agent can also be in micro-encapsulated form with one or more excipients as noted above. The solid dosage forms of tablets, dragées, capsules, pills, and granules can be prepared with coatings and shells such as enteric coatings, release controlling coatings and other coatings well known in the pharmaceutical formulating art. In such solid dosage forms, the agent can be admixed with at least one inert diluent such as sucrose, lactose and starch. Such dosage forms can also comprise, as in normal practice, additional substances other than inert diluents, e.g., tableting lubricants and other tableting aids such as magnesium stearate and microcrystalline cellulose. In the case of capsules, tablets and pills, the dosage forms can also comprise buffering agents. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Pharmaceutical compositions include formulations suitable for oral administration may be provided as discrete units, such as tablets, capsules, cachets, syrups, elixirs, prepared food items, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Accordingly, formulations suitable for rectal administration include gels, creams, lotions, aqueous or oily suspensions, dispersible powders or granules, emulsions, dissolvable solid materials, douches, and the like can be used. The formulations are preferably provided as unit-dose suppositories comprising the active ingredient in one or more solid carriers forming the suppository base, for example, cocoa butter. Suitable carriers for such formulations include petroleum jelly, lanolin, polyethyleneglycols, alcohols, and combinations thereof. Alternatively, colonic washes with the rapid recolonization deployment agent of the present disclosure can be formulated for colonic or rectal administration.

### Administration

In some embodiments of any of the aspects, the methods described herein relate to treating a subject having or diagnosed as having an autoimmune disease (e.g., multiple sclerosis) comprising administering an agent that targets CXCR6 as described herein. In some embodiments, the methods described herein relate to treating a subject having or diagnosed as having an autoimmune disease comprising administering an agent that inhibits SerpinB1 as described herein. Subjects having an autoimmune disease (e.g., multiple sclerosis) can be identified by a physician using current methods of diagnosing a condition. Symptoms and/or complications of an autoimmune disease, which characterize this disease and aid in diagnosis are well known in the art and include but are not limited to, blurred or double vision, loss of coordination, muscle tremors, or numbness in limbs. Tests that may aid in a diagnosis of, e.g. an autoimmune disease, include but are not limited to MRI to look for lesions in the brain and are known in the art for a given autoimmune disease. A family history of an autoimmune disease will also aid in determining if a subject is likely to have the condition or in making a diagnosis of an autoimmune disease.

The agents described herein (e.g., an agent that targets CXCR6, or an agent that inhibits SerpinB1) can be administered to a subject having or diagnosed as having an autoimmune disease (e.g., multiple sclerosis). In some embodiments, the methods described herein comprise administering an effective amount of an agent to a subject in order to alleviate at least one symptom of the autoimmune disease. As used herein, “alleviating at least one symptom of the autoimmune disease” is ameliorating any condition or symptom associated with the autoimmune disease (e.g., muscle tremors, bladder issues, numbness in limbs, double vision). As compared with an equivalent untreated control, such reduction is by at least 5%, 10%, 20%, 40%, 50%, 60%, 80%, 90%, 95%, 99% or more as measured by any standard technique. A variety of means for administering the agents described herein to subjects are known to those of skill in the art. In one embodiment of any of the aspects, the agent is administered systemically or locally (e.g., to the brain, or other affected organ, e.g., the colon).

In one embodiment of any of the aspects, the agent is administered intravenously. In one embodiment of any of the aspects, the agent is administered continuously, in intervals, or sporadically. The route of administration of the agent will be optimized for the type of agent being delivered (e.g., an antibody, a small molecule, an RNAi), and can be determined by a skilled practitioner.

The term “effective amount” as used herein refers to the amount of an agent (e.g., an agent that targets CXCR6, or an agent that inhibits SerpinB1) can be administered to a subject having or diagnosed as having an autoimmune disease (e.g., multiple sclerosis) needed to alleviate at least one or more symptom of an autoimmune disease. The term “therapeutically effective amount” therefore refers to an amount of an agent that is sufficient to provide a particular anti-autoimmune disease effect when administered to a typical subject. An effective amount as used herein, in various contexts, would also include an amount of an agent sufficient to delay the development of a symptom of the autoimmune disease, alter the course of a symptom of an autoimmune disease (e.g., slowing the progression of muscle tremors, bladder issues, numbness in limbs, double vision), or reverse a symptom of the autoimmune disease (e.g., correcting vision, halting muscle tremors, or correcting bladder issues). Thus, it is not generally practicable to specify an exact “effective amount”. However, for any given case, an appropriate “effective amount” can be determined by one of ordinary skill in the art using only routine experimentation.

In one embodiment of any of the aspects, the agent is administered continuously (e.g., at constant levels over a period of time). Continuous administration of an agent can be achieved, e.g., by epidermal patches, continuous release formulations, or on-body injectors.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

### Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment of any of the aspects, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

The effective dose can be estimated initially from cell culture assays. A dose can be formulated in animals. Generally, the compositions are administered so that a compound of the disclosure herein is used or given at a dose from 1 μg/kg to 1000 mg/kg; 1 μg/kg to 500 mg/kg; 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. Further contemplated is a dose (either as a bolus or continuous infusion) of about 0.1 mg/kg to about 10 mg/kg, about 0.3 mg/kg to about 5 mg/kg, or 0.5 mg/kg to about 3 mg/kg. It is to be further understood that the ranges intermediate to those given above are also within the scope of this disclosure, for example, in the range 1 mg/kg to 10 mg/kg, for example use or dose ranges such as 2 mg/kg to 8 mg/kg, 3 mg/kg to 7 mg/kg, 4 mg/kg to 6 mg/kg, and the like.

### Combinational Therapy

In one embodiment of any of the aspects, the agent described herein is used as a monotherapy. In another embodiment of any of the aspects, the agents described herein can be used in combination with other known agents and therapies for an autoimmune disease. Administered “in combination,” as used herein, means that two (or more) different treatments are delivered to the subject during the course of the subject's affliction with the disorder, e.g., the two or more treatments are delivered after the subject has been diagnosed with the disorder (an autoimmune disease) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Therapeutics currently used to treat an autoimmune disease include, but are not limited to, mitoxantrone, interferon β1a therapy, peginterferon beta 1a, azathioprine, fingolimod, natalizumab, glatiramer, steroids (e.g. prednisolone, methylprednisolone, cortisone, hydrocortisone, budesonide,), analgesics and anti-inflammatory drugs (e.g. capsaicin, acetaminophen, ibuprofen, mesalamine), sulfasalazine, oxycodone, methotrexate, azathioprine, adalimumab, infliximab, mercaptopurine, hydroxychloroquine, antibiotics (e.g. clindamycin, metronidazole, aminosalicylic acid, penicillin), vitamins (vitamin D, vitamin B12), immunosuppressive agents, mycophenolate, FK506, antibodies, immunoablative agents such as CAMPATH, anti-CD3 antibodies or other antibody therapies, cytoxin, fludarabine, rapamycin, mycophenolic acid, steroids, FR901228, cytokines, or a peptide vaccine, such as that described in Izumoto et al. 2008 J Neurosurg 108:963-971, which is incorporated herein by reference in its entirety.

When administered in combination, the agent and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of an autoimmune disease) is lower (e.g., at least 20%, at least 30%, at least 40%, or at least 50% lower) than the amount or dosage of each agent individually required to achieve the same therapeutic effect.

### Parenteral Dosage Forms

Parenteral dosage forms of an agents described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. Without limitations, oral administration can be in the form of solutions, suspensions, tablets, pills, capsules, sustained-release formulations, oral rinses, powders and the like.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Controlled and Delayed Release Dosage Forms

In some embodiments of any of the aspects described herein, an agent is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Chemg-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy

The efficacy of an agents described herein, e.g., for the treatment of an autoimmune disease, can be determined by the skilled practitioner. However, a treatment is considered “effective treatment,” as the term is used herein, if one or more of the signs or symptoms of the autoimmune disease are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., vision, bladder function, muscle stability, and feeling in limbs. Efficacy can also be measured by a failure of an individual to worsen as assessed by hospitalization, or need for medical interventions (i.e., progression of the muscle tremors, bladder issues, numbness in limbs, double vision). Methods of measuring these indicators are known to those of skill in the art and/or are described herein.

Efficacy can be assessed in animal models of a condition described herein, for example, a mouse model or an appropriate animal model of autoimmune or inflammatory disease, as the case may be. When using an experimental animal model, efficacy of treatment is evidenced when a statistically significant change in a marker is observed, e.g., a slowing of muscle tremors, bladder issues, numbness in limbs, double vision.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Some embodiments of the various aspect described herein can be described as in the following paragraphs:
1. A method for treating an autoimmune disease, comprising administering to a subject having an autoimmune disease an agent that targets CXCR6; wherein targeting CXCR6 results in the depletion of a cell expressing CXCR6 or population thereof.
2. A method for treating an autoimmune disease, comprising administering to a subject having an autoimmune disease an agent that inhibits SerpinB1.
3. The method of paragraph 1, wherein the cell population is depleted by at least 10%, at least 20%, at least 30%, at least 40%, at least 50%, at least 60%, at least 70%, at least 80%, at least 90% or more as compared to an appropriate control.
4. The method of paragraph 1, wherein the cell population is a Th17 or Th17-derived cell population.
5. The method of paragraph 1, wherein the agent that targets CXCR6 is linked to at least a second agent.
6. The methods of paragraphs 1-2, wherein the autoimmune disease is selected from the list consisting of Rheumatoid arthritis, Crohn's disease, lupus, celiac disease, Sjogren's syndrome, polymyalgia rheumatic, multiple sclerosis, ankylosing spondylitis, type 1 diabetes, alopecia areata, vasculitis, autoimmune uveitis, juvenile idiopathic arthritis, and temporal arteritis.
7. The method of paragraphs 1-2, wherein the autoimmune disease is multiple sclerosis.
8. The method of paragraphs 1-2, wherein the subject is human.
9. The method of paragraphs 1, wherein the agent that targets CXCR6 is selected from the group consisting of a small molecule, an antibody, and a peptide.
10. The method of paragraph 2, wherein the agent that inhibits SerpinB1 is selected from the group consisting of a small molecule, an antibody, a peptide, a genome editing system, an antisense oligonucleotide, and an RNAi.
11. The method of paragraphs 8-9, wherein the antibody is a depleting antibody.
12. The method of paragraph 9, wherein the RNAi is a microRNA, an siRNA, or a shRNA.
13. The method of paragraph 2, wherein inhibiting SerpinB1 is inhibiting the expression level and/or activity of SerpinB1.
14. The method of paragraph 12, wherein the expression level and/or activity of SerpinB1 is inhibited by at least 50%, at least 60%, at least 70%, at least 80%, at least 90%, or more as compared to an appropriate control.
15. A method for selecting a population of Th17 cells or Th17-derived cells, the method comprising measuring the level of CXCR6 in a population of candidate cells, and selecting cells which exhibit expression of CXCR6.
16. The method of paragraphs 14, wherein the level of CXCR6 is increased by at least 2-fold, at least 3-fold, at least 4-fold, at least 5-fold, at least 6-fold, at least 7-fold, at least 8-fold, at least 9-fold, at least 10-fold, or more as compared to a reference level.
17. A method of treating an autoimmune disease, the method comprising:
   a. receiving the results of an assay that indicate an increase in the levels of CXCR6 in a biological sample from a subject compared with an appropriate control; and
   b. administering to the subject an agent that inhibits the level or activity of SerpinB1.
18. The method of paragraph 17, wherein the assay is flow cytometry, reverse transcription-polymerase chain reaction (RT-PCR), RNA sequencing, or immunohistochemistry.
19. The method of paragraph 17, wherein the subject is suspected of having, or has an autoimmune disease.
20. The method of paragraph 17, further comprising, detecting the levels of SerpinB1 expressed by Th17 cells in a subject; and receiving the results of an assay that indicate an increase in SerpinB1 levels compared with an appropriate control.
21. The method of paragraph 17, further comprising, detecting the levels of one or more of: perforin-A, granzyme A (GzmA), GzmC, interleukin-17 (IL-17), IL-6, IL-21, IL-23, interleukin-23 receptor (IL-23R), IL-7Rα and IL-1R1, interferon gamma (IFNγ), RAR Related Orphan Receptor C (Rorc), and granulocyte-macrophage colony-stimulating factor (GM-CSF) in the subject.
22. The method of paragraph 17, further comprising, detecting leukocyte accumulation in the spinal cord.
23. The method of paragraph 17, wherein the autoimmune disease is selected from the group consisting of: rheumatoid arthritis, Crohn's disease, lupus, celiac disease, Sjogren's syndrome, polymyalgia rheumatic, multiple sclerosis, ankylosing spondylitis, type 1 diabetes, alopecia areata, vasculitis, autoimmune uveitis, juvenile idiopathic arthritis, and temporal arteritis.
24. The method of paragraph 17, wherein the autoimmune disease is multiple sclerosis.
25. The method of paragraph 17, wherein the subject is human.
26. The method of paragraph 17, prior to receiving the results of an assay in step (a), obtaining a biological sample from the subject.
27. The method of paragraph 17, wherein the biological sample is synovial fluid, spinal fluid, tissue, or blood.
28. A method of decreasing a population of T cells expressing CXCR6, the method comprising: administering an agent that decreases the levels or activity of SerpinB1 in leukocytes.
29. The method of paragraph 28, wherein the said decreasing the levels or activity of SerpinB1 in leukocytes comprises administering an inhibitor of SerpinB1.
30. The method of paragraph 28, wherein the T cell population is depleted by at least 10%, at least 20%, at least 30%, at least 40%, at least 50%, at least 60%, at least 70%, at least 80%, at least 90% or more as compared to an appropriate control.
31. The method of paragraph 28, wherein the T cell population is a Th17 or Th17-derived cell population.
32. The method of paragraph 28, wherein said decreasing levels or activity of SerpinB1 is in a subject in need of treatment for an autoimmune disease.
33. The method of paragraph 32, wherein the autoimmune disease is selected from the group consisting of: rheumatoid arthritis, Crohn's disease, lupus, celiac disease, Sjogren's syndrome, polymyalgia rheumatic, multiple sclerosis, ankylosing spondylitis, type 1 diabetes, alopecia areata, vasculitis, autoimmune uveitis, juvenile idiopathic arthritis, and temporal arteritis.
34. The method of paragraph 32, wherein the autoimmune disease is multiple sclerosis.
35. The method of paragraph 28, wherein the agent is selected from the group consisting of: a small molecule, an antibody, a peptide, a genome editing system, an antisense oligonucleotide, and an RNAi.
36. The method of paragraph 35, wherein the antibody is a depleting antibody.
37. The method of paragraph 35, wherein the RNAi is a microRNA, an siRNA, or a shRNA.
38. The method of paragraph 28, wherein the level and/or activity of SerpinB1 is inhibited by at least 50%, at least 60%, at least 70%, at least 80%, at least 90%, or more as compared to an appropriate control.
39. The method of paragraph 28, wherein the administering inhibits inflammation.
40. The method of paragraph 28, wherein the administering inhibits leukocyte accumulation in the spinal cord.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1: CD4 Cell Sb1 Executes Late-Occurring Step Crucial for Accumulation of Pathogenic Cells in the CNS

At mucosal surfaces, IL-17+ helper T cells (Th17 cells) and the cytokines IL-17 and IL-22 provide protection from fungal and bacterial infections, but in autoimmune settings, Th17 cells are converted into polyfunctional cells (also called ex-Th17, Th17/Th1, non-classical Th1 cell) that produce IFNγ and GM-CSF and are pathogenic agents of multiple sclerosis (MS). Work described herein indicate that polyfunctional CD4 T cell populations arise in MS and, on expansion, accumulate as pathogenic T cells in the central nervous system (CNS). Also provided herein are data that describe the molecular mechanism that controls expansion of wild type polyfunctional CD4 T cells and prevents serpinb1-deficient Th17 derived polyfunctional cells from expanding and accumulating as pathogenic T cells in the CNS.

SerpinB1 (sb1) had been defined earlier as (i) a protease inhibitor, (ii) an ancient member of a family of regulatory genes, and (iii) a top Th17 signature gene. It was shown that Th17 cell generation is negatively regulated by SerpinB1 (Hou, 2015). However, upon immunization with MOG, sb1−/− mice are largely resistant to EAE (Hou, 2016) and, consistent with disease attenuation, have a deficit of CD4 cells in the CNS. Adoptive transfer established that disease attenuation and lack of CD4 cells in the CNS are both due to sb1 deletion in CD4 T cells. Also shown herein are data indicating that deletion of sb1 in CD4 cells does not alter expression/responsiveness of IL-23R, IL-1R, and IL-7R, nor does it affect IL17 production, antigen recall response, CD4 cell proliferation or metabolic changes. These data indicate that early events of autoimmune Th17 cell generation proceed normally in sb1 deficient mice. The data strongly indicate that CD4 cell sb1 executes a late-occurring step crucial for accumulation of pathogenic cells in the CNS.

Having eliminated upstream steps, effector (CD44+) CD4 cells in lymph node at disease onset were focused on. It was found that sb1-deficient effector CD4 cells differentiate still further to become IFNγ+ and GM-CSF+ CD4 cells, a key function required for pathogenicity, but the number of these cells is decreased compared to wild type mice. The finding that polyfunctional effector CD4 cells in sb1-deficient mice have all other properties of wild-type polyfunctional CD4 cells, yet don't accumulate in substantial numbers in the CNS and cause disease, indicated that comparative gene expression analysis of sb1−/− and WT CD4 effector cells, in particular their different frequency, could be leveraged to identify other genes expressed in IFNγ+ GM-CSF+ polyfunctional CD4 cells and possibly explain the different fates of these cells in EAE.

By the use of RNA sequencing, additional genes co-expressed in the IFNγ+ and GM-CSF+ cells were identified. It was found that a granzyme, likely granzyme-C (gzmC), a serine protease not present in other CD4 cells in EAE, mediates proliferation-induced cell death and is specifically inhibited by serpinB1. Also demonstrated herein are the findings that sb1 inhibits gzmC and that the pathogenic CD4 cells are “polyfunctional”—not only producing multiple cytokines and particularly GM-CSF, but also containing “cytolytic” granules, proliferating extremely rapidly, and auto-regulating an activation-induced death mechanism that determines the extent of population expansion. Through the cell death mechanism, the opposing actions of a granule protease and serpinB1 appear to control the extent of expansion of polyfunctional pathogenic CD4 cells.

Importantly, a surface protein was also identified, the chemokine receptor CXCR6, that specifically “marks” the cytolytic granule-containing multiple cytokine-producing pathogenic CD4 cells in myelin peptide-immunized mice.

It is specifically contemplated herein to utilize an anti-human CXCR6 antibody to deplete (e.g., kill, or neutralize its activity, or inactivate the cell) pathogenic CD4 cells in subjects with MS and other autoimmune disorders. When studied in the mouse model, CXCR6−/− animals develop EAE similar to CXCR6+ (wild type) mice (Kim, 2009), indicating that CXCR6 does not exercise any required function such as chemotactic responses in EAE disease. Thus data presented herein indicate that the role of CXCR6 in EAE is strictly as a trackable marker of pathogenic CD4 cells that renders the cells amenable to direct study and manipulation.

It is determined that CXCR6+ CD4 cells are a small subset of total CD4 cells in the draining lymph nodes in EAE mice, but are the major CD4 cells infiltrated in the spinal cord, indicating that CXCR6 identifies polyfunctional pathogenic CD4 cells.

Functional participation of proteins encoded by key signature genes in CXCR6+ pathogenic CD4 T cells is demonstrated. Participation of cytolytic granules and their granzymes, likely including granzyme C, as inducers of cell death and of SerpinB1 with opposing action supporting cell survival and preventing cell death constitute the cell death mechanism that determines the extent of expansion of the CXCR6+ pathogenic CD4 cells is investigated.

It is determined herein that CXCR6 serves as a trackable marker of pathogenic CD4 cells also in a model of mouse delayed type hypersensitivity (DTH). DTH reaction is mediated by CD4 cell activation and is the prototype of many cellular immune-mediated autoimmune responses, such as rheumatoid arthritis, multiple sclerosis, contact dermatitis, inflammatory bowel disease, autoimmune myocarditis, type 1 diabetes, et al. The data indicate that the serpinb1-granule protease cell death mechanism and the effect of SerpinB1 in regulating the size and thus destructive capacity of the pathogenic CXCR6+ population, operate also in the DTH model, consistent with the mechanism operating not only in EAE/MS but extending to other autoimmune and autoimmune-like disorders.

Using the system in which WT B6 mice are transferred with OT-II cells and then immunized with ovalbumin peptide (OVA), data presented herein show that CXCR6 identifies the polyfunctional OT-II cells that produce cytokines IL-17 and GM-CSF. These data demonstrate that CXCR6 broadly marks polyfunctional T cells in other Th17-driven disorders at autoimmune settings.

It is additionally determined herein that human CXCR6+ cells, which represent a minor population in circulating blood of healthy controls, are present in large numbers in inflammatory synovial fluids of patients with the autoimmune disorder rheumatoid arthritis and that CXCR6 is suitable for use as a trackable marker to identify and quantify pathogenic CD4 cells producing cytokine IL-17, IFNγ, and GM-CSF in this autoimmune disorder.

It is additionally determined herein that delivery of anti-mouse CXCR6 monoclonal antibody, but not isotype control antibody, to myelin peptide-immunized mice prevents the development of EAE disease. All symptoms of disease are prevented by the CXCR6 antibody treatment.

It is also determined herein that treatment of mice that have already developed EAE symptoms (mean disease score of 2) with anti-mouse CXCR6 antibody completely stops further disease development and further weight loss and reverses the disease symptoms. Consistent with the absence of disease symptoms after anti-CXCR6 antibody treatment, the treated mice fail to accumulate pathogenic CD4 cells in the spinal cord.

Multiple mechanisms could be envisioned to explain how anti-CXCR6 antibody prevents EAE and is effective as a treatment for the disease. Since CXCR6 is a chemokine receptor, without wishing to be bound by a particular theory, one might hypothesize that a CXCR6 antibody would block chemoattraction of CXCR6+ cells to the spinal cord and thereby block disease. Were that the case, the effective treatment with anti-CXCR6 antibody would increase the number of target cells remaining in the lymph node. It is however determined herein that treatment of EAE model mice with anti-mouse CXCR6 antibody for 24 hours not only failed to increase, but actually decreased, the number of IL-17+GM-CSF+ and GM-CSF-single positive CD4 cells in the lymph node. These data eliminate mechanism models invoking blocking chemoattraction to the spinal cord. Rather the data indicate a straightforward mechanism in which anti-CXCR6 antibody causes depletion of the polyfunctional T cells.

Findings presented herein have opened a new study area by making the pathogenic CD4 cells amenable to direct detailed molecular characterization and manipulation. It is significant that the CXCR6 expression and the SerpinB1 regulated events occur at a very late stage in EAE pathogenesis, and therefore targeting this pathway in MS would not adversely affect other protective CD4 cell functions. Th17 mediated protection from fungal and bacterial infection, for example, would not be affected. Finally, these proposed findings are relevant not only for MS, but also for other aggressive autoimmune disorders.

### Example 2: Cxcr6 Expression and the Serpinb1 Regulate EAE Pathogenesis

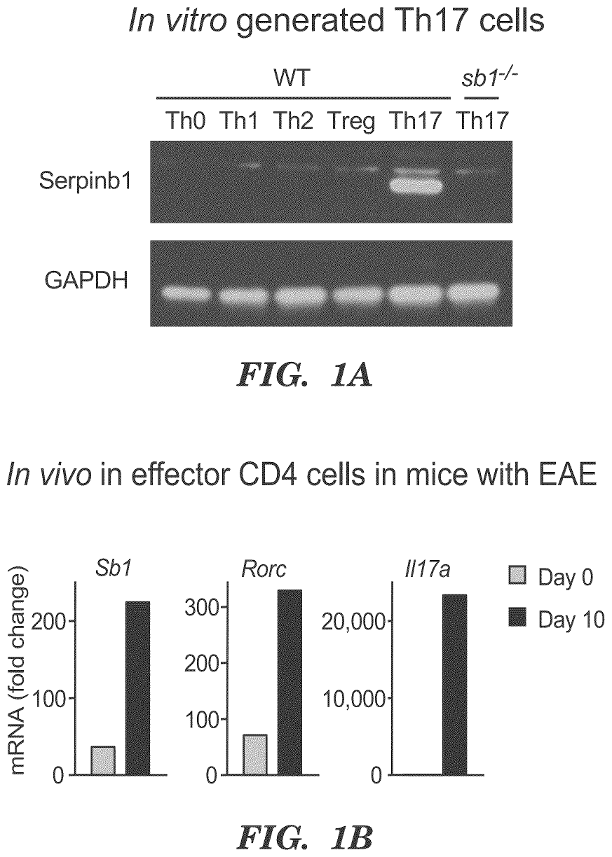

FIGS. 1A and 1B present data that show Serpinb1 (Sb1), a protease inhibitor, is a signature gene of Th17 cells. (FIG. 1A) Western blot showing SerpinB1 levels in Th17 cells. (FIG. 1B) mRNA levels of indicated gene in effector CD4 cells in EAE (Day 10) and naïve (Day 0) mice.

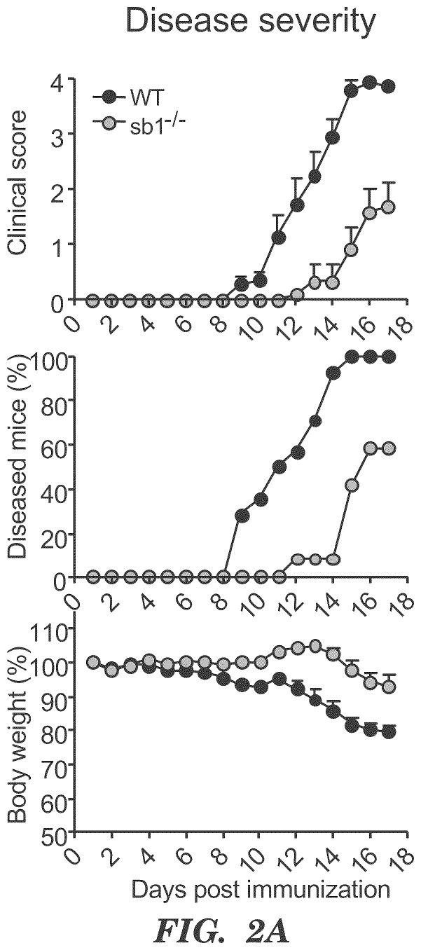

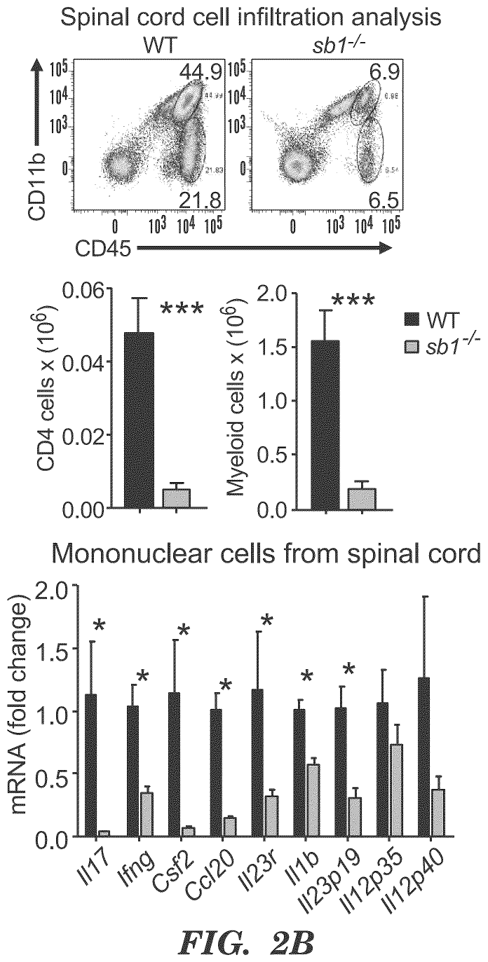

FIGS. 2A and 2B present data that show EAE in mice with global deletion of Serpinb1 (Sb1^{-/-}). (FIG. 2A) Characteristics of disease in indicated mouse. (FIG. 2B) Characterization of spinal cord cells in indicated mouse. Sb1 is essential for pathogenicity of EAE. Sb1 is essential for CNS infiltration of CD4 cells

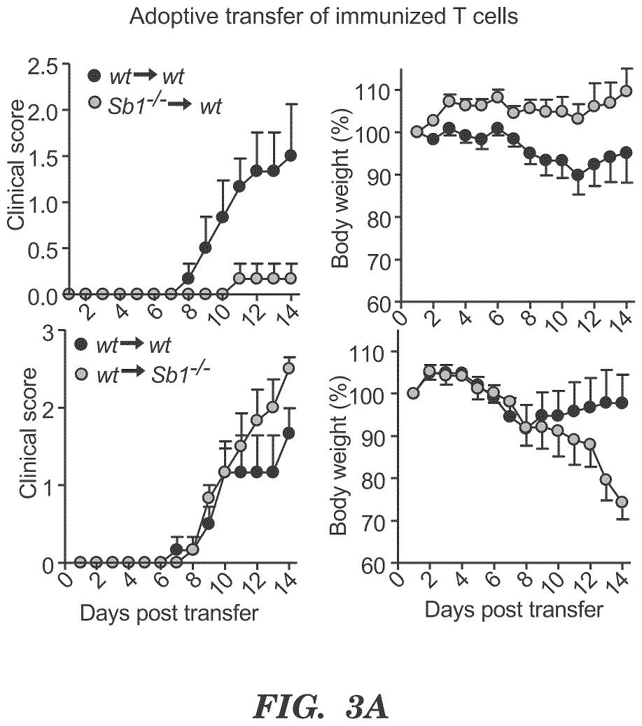

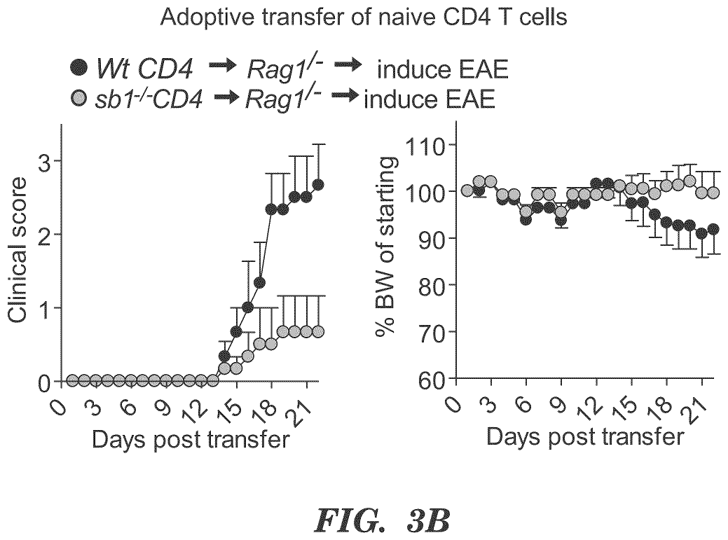

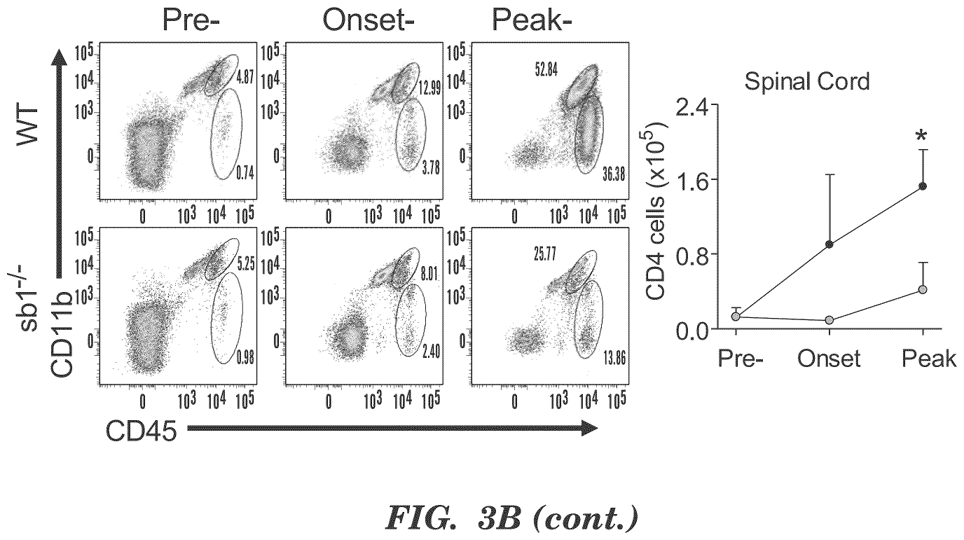

FIGS. 3A and 3B present data that show EAE in two models of Sb1 deletion in T cells. (FIG. 3A) Adoptive transfer of CD4 T cells recovered from immunized wild-type or sb1^{-/-} mice into naïve WT mice (top) or from WT mice into naïve WT or sb1^{-/-} mice. (FIG. 3B) Transfer of naive CD4 T cells from naive wild-type or sb1^{-/-} mice into Rag^{-/} mice that were then immunized to induce EAE. Disease amelioration only requires serpinb1 deletion in T cells or only in CD4 T cells.

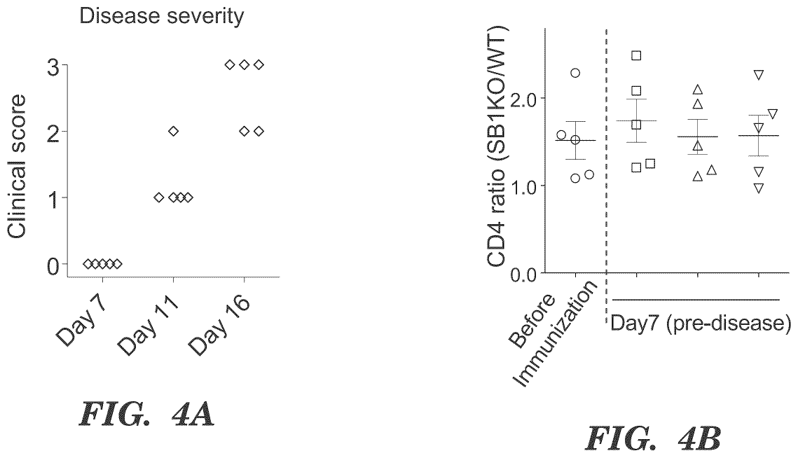

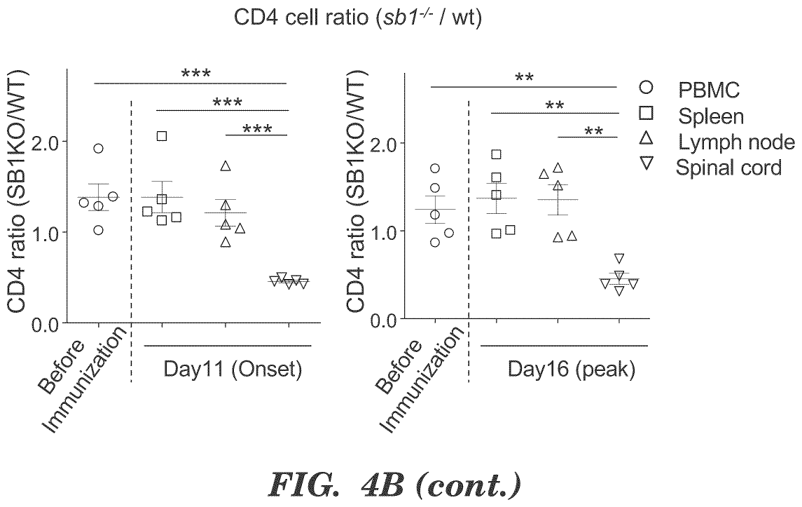

FIGS. 4A and 4B present data that show EAE in Sb1^{-/-}:WT mixed chimeric mice. (FIG. 4A) Clinical score depicting disease severity. (FIG. 4B) CD4 cell ratio at indicated time points, and in various organs. Sb1^{-/-} CD4 cells are preferentially depleted in the spinal cord.

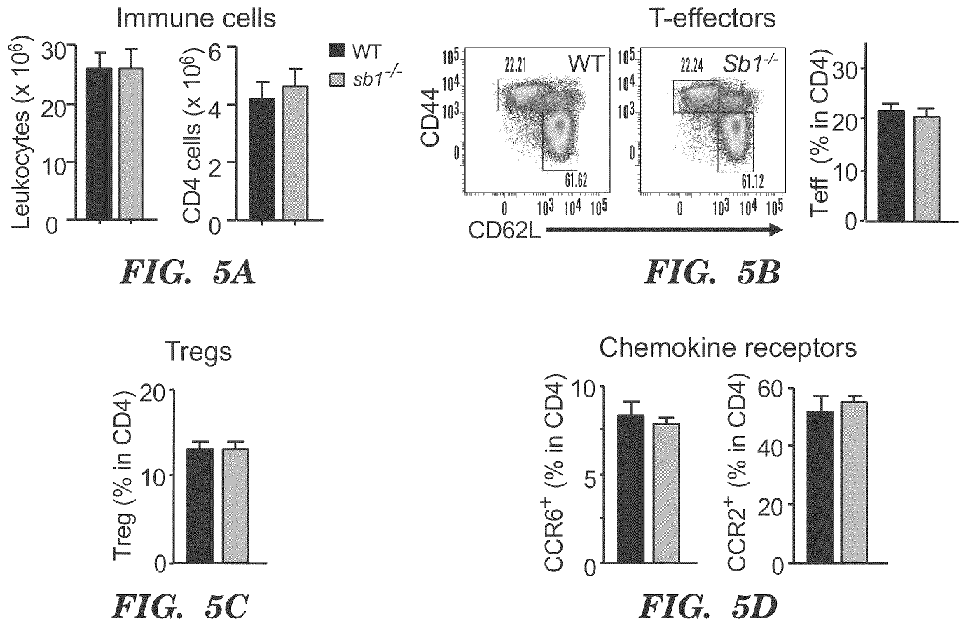

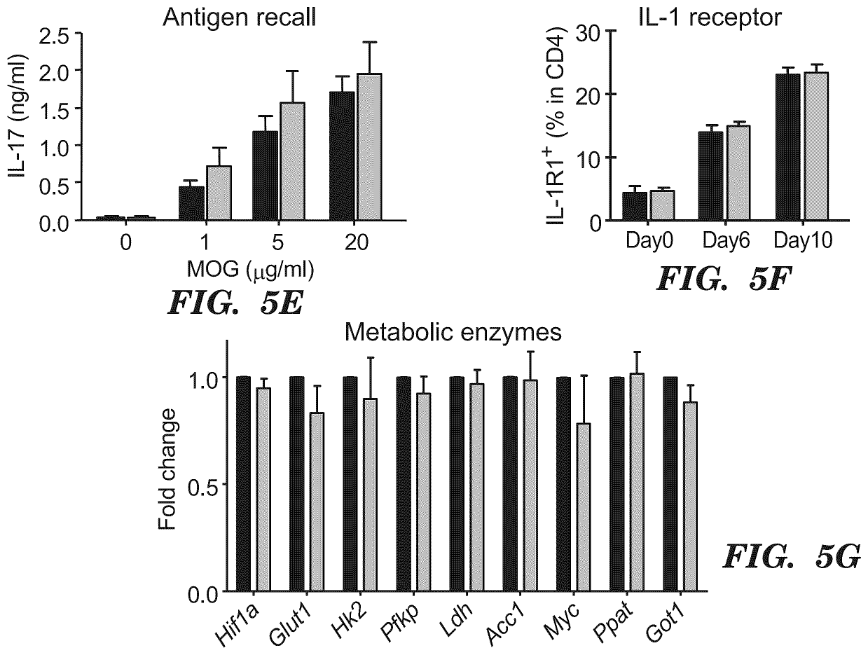

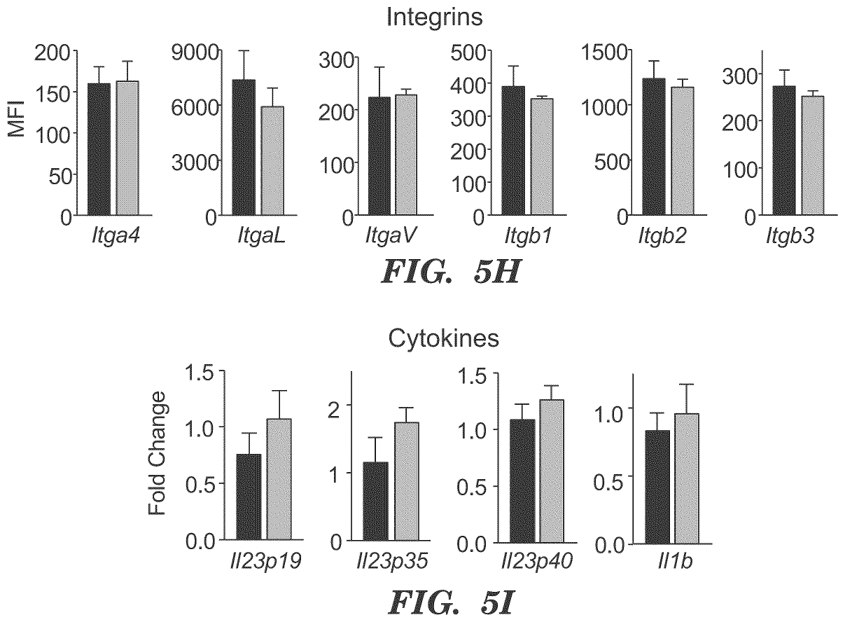

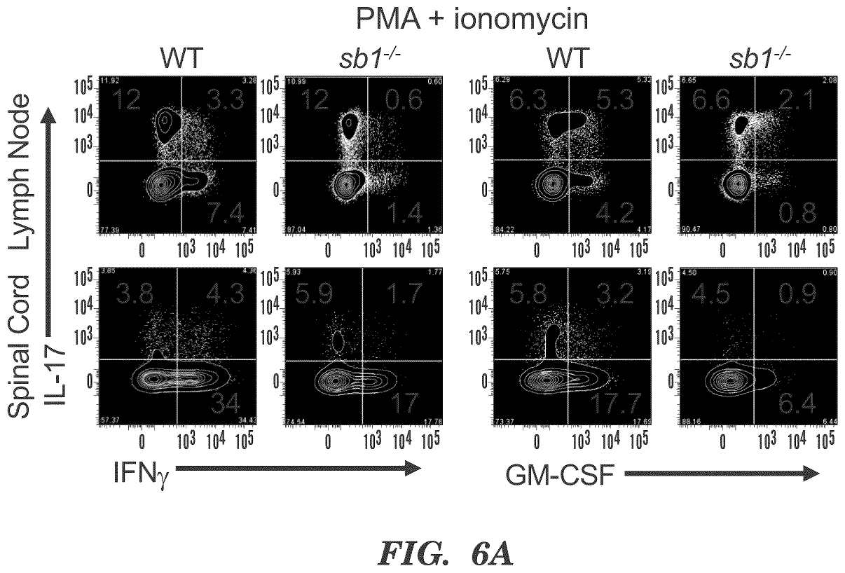

FIGS. 5A-5I present data that show CD4 cell differentiation to Th17 cells in peripheral lymphoid organs. Quantification of (FIG. 5A) immune cells, (FIG. 5B) T-effectors, (FIG. 5C) T regulatory cells (Tregs), (FIG. 5D) chemokine receptors, (FIG. 5E) antigen recall and IL-17 production, (FIG. 5F) IL-1 receptor, (FIG. 5G) metabolic enzymes, (FIG. 5H) integrins, and (FIG. 5I) cytokines in a wild-type (black bar) or Sb1^{-/-} (red bar) mouse. Serpinb1 is not required to generate antigen-specific IL-17⁺ CD4 effector cells.

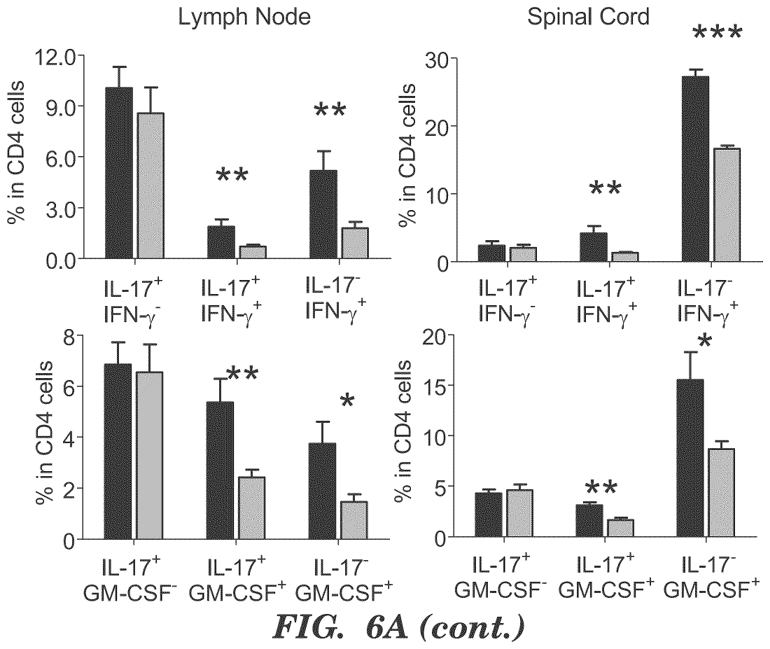

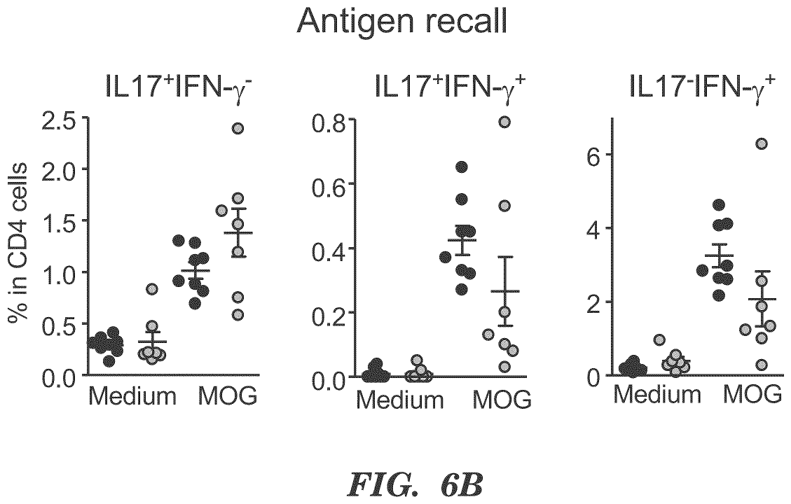

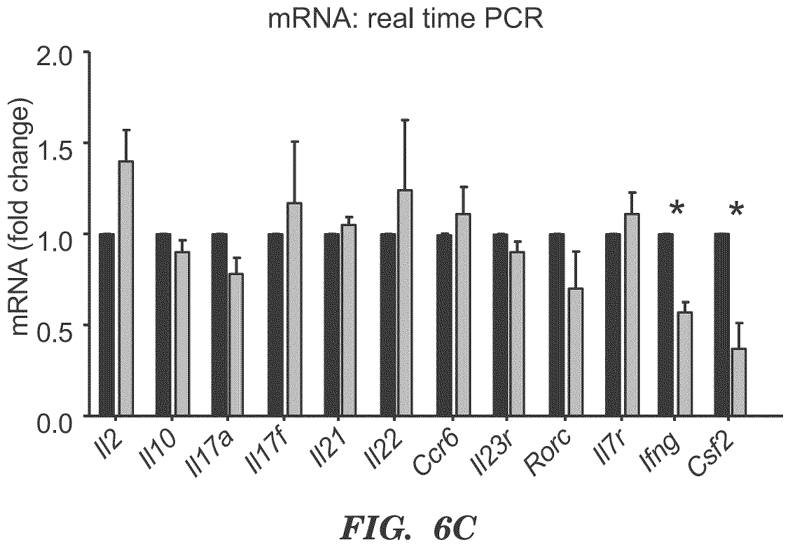

FIGS. 6A-6C present data that show IFNγ+ and GM-CSF+ effector CD4 cells in WT and Sb1^{-/-} mice. (FIG. 6A) Quantification of various cytokine-producing CD4 effector cells following PMA plus ionomycin. (FIG. 6B) Quantification of antigen recall. (FIG. 6C) mRNA levels in lymph node effector CD4 cells quantified by real time PCR. Red symbols indicate Sb1^{-/-}; black indicate WT^{.-}IFNγ⁺ and GM-CSF⁺ effector CD4 cells are decreased in Sb1^{-/-} mice.

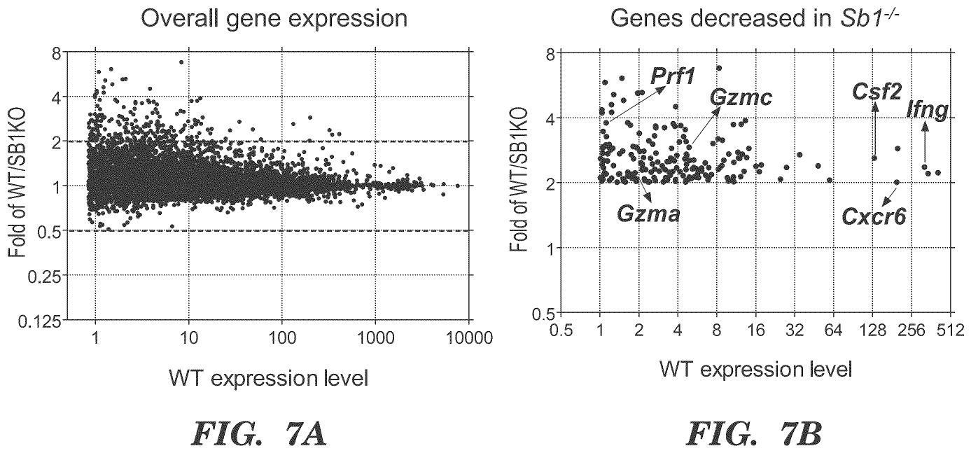

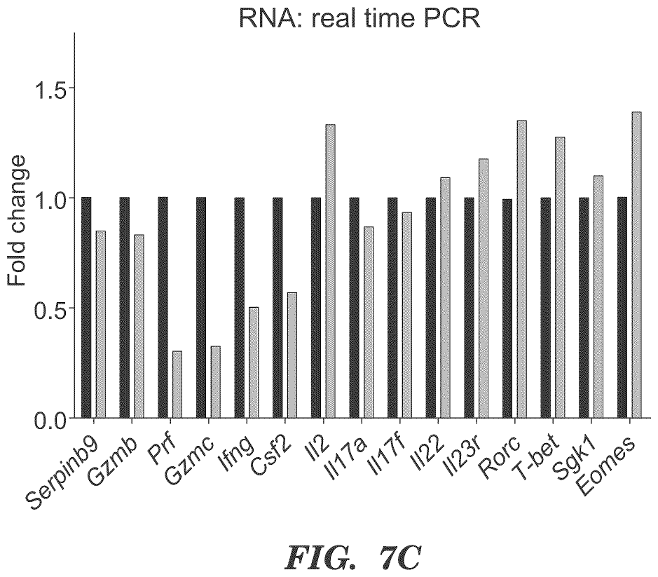

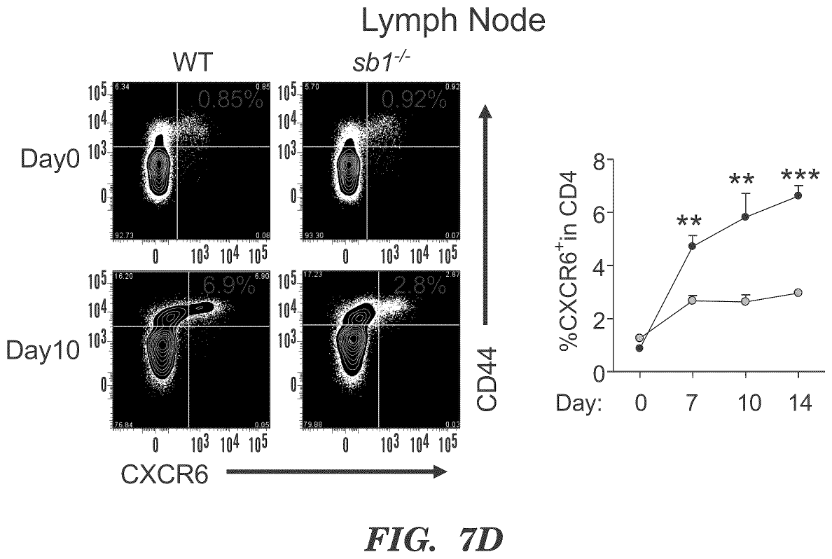

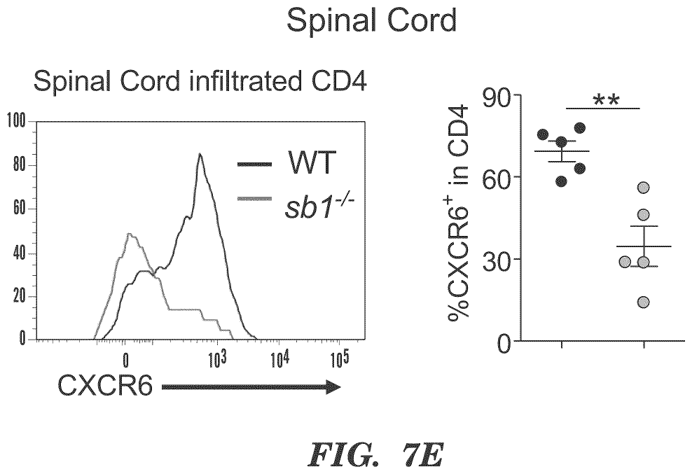

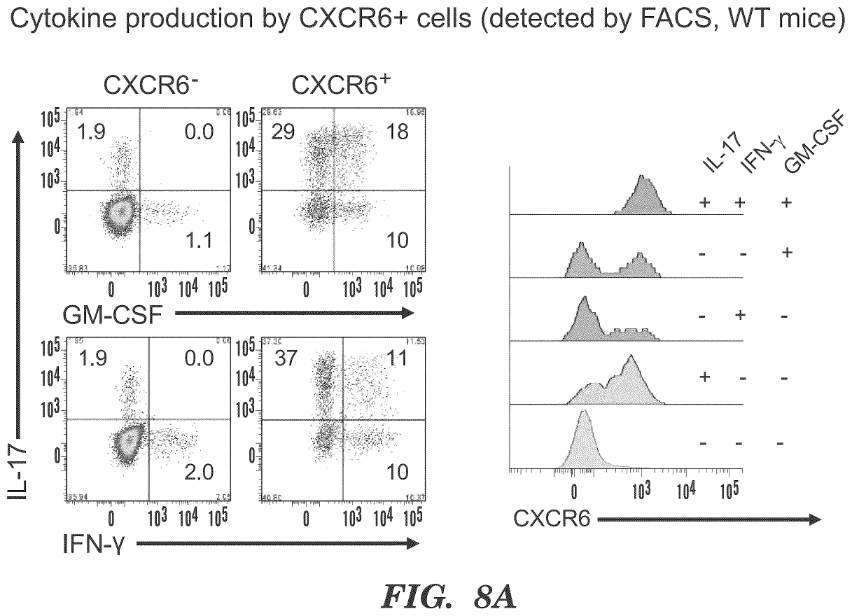

FIGS. 7A-7E present data that identify genes differentially expressed in Sb1^{-/-} and WT CD4 effector cells. (FIG. 7A) Expression level of 9649 genes determined by RNA sequencing of CD4 effector cells of indicated mice. (FIG. 7B) The 218 genes with expression decreased by a factor of 2 or more in Sb1^{-/-} compared with WT mice. (FIG. 7C) mRNA levels quantified by real time PCR. (FIG. 7D) CXCR6 expression in CD4 cells in indicated mouse. (FIG. 7E) CXCR6 expression in spinal cord infiltrated CD4 cells. Red symbols indicate Sb1^{-/-}; black indicate WT. Genes underrepresented in CD4 effector cells of sb1^{-/-} mice encode IFNγ (Ifng) and GM-CSF (csf2) (as anticipated) and also cell surface CXCR6 (Cxcr6), the granule protease granzyme-C (Gzmc) and the pore-forming granule protein perforin (Pfr1).

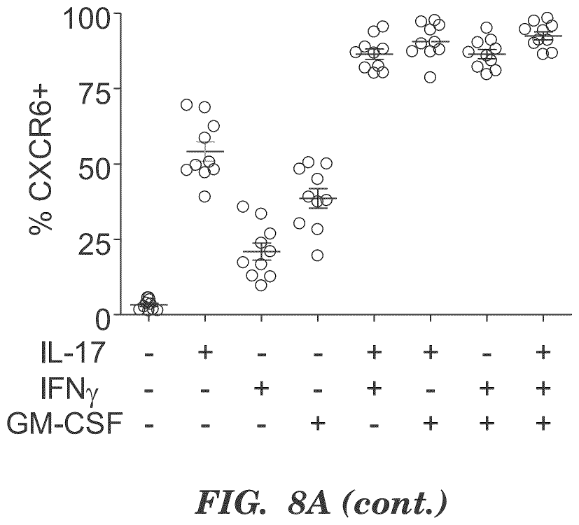

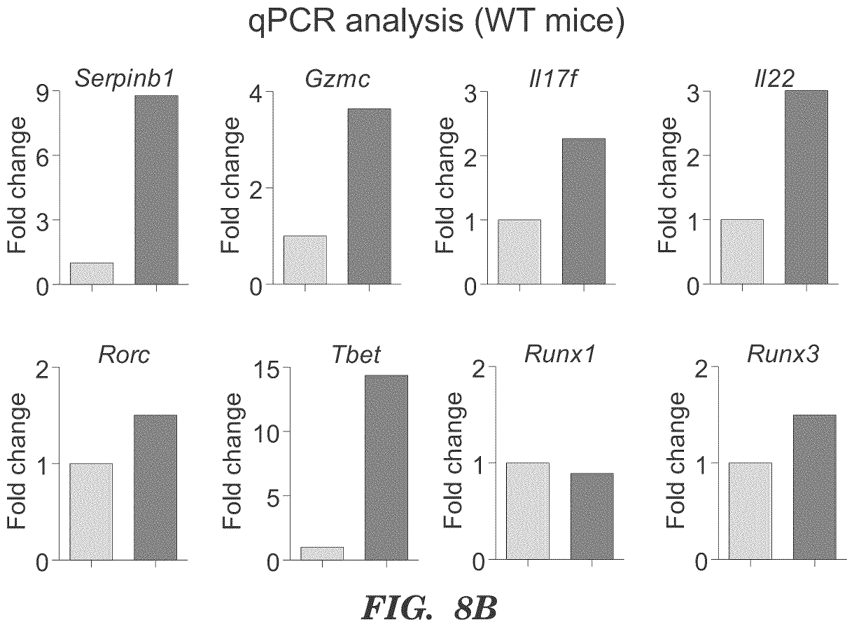

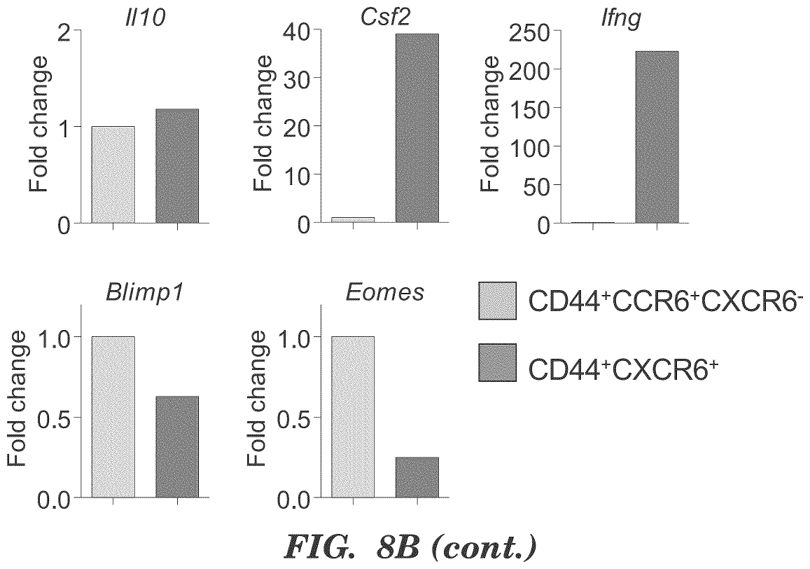

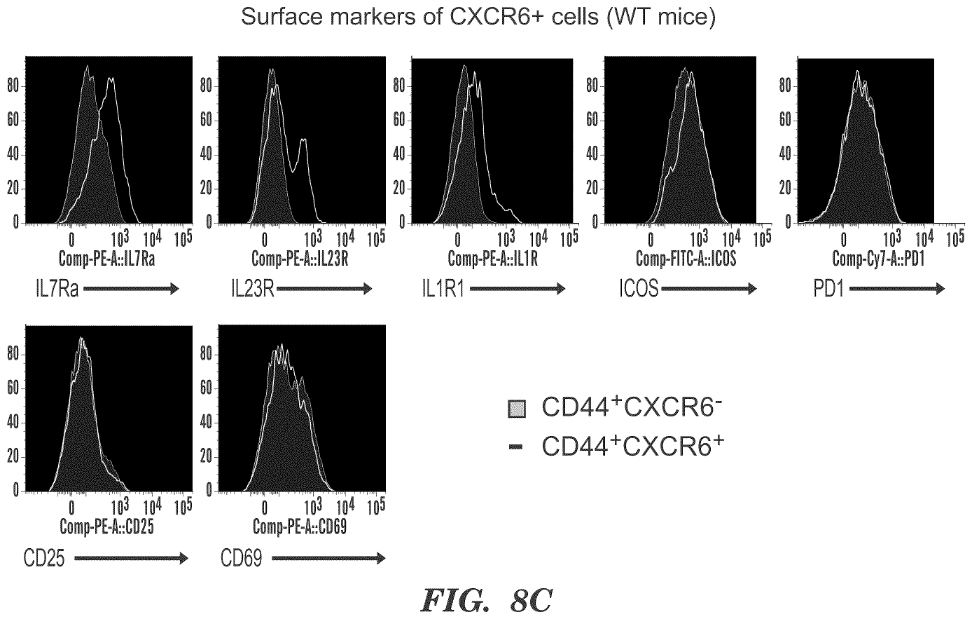

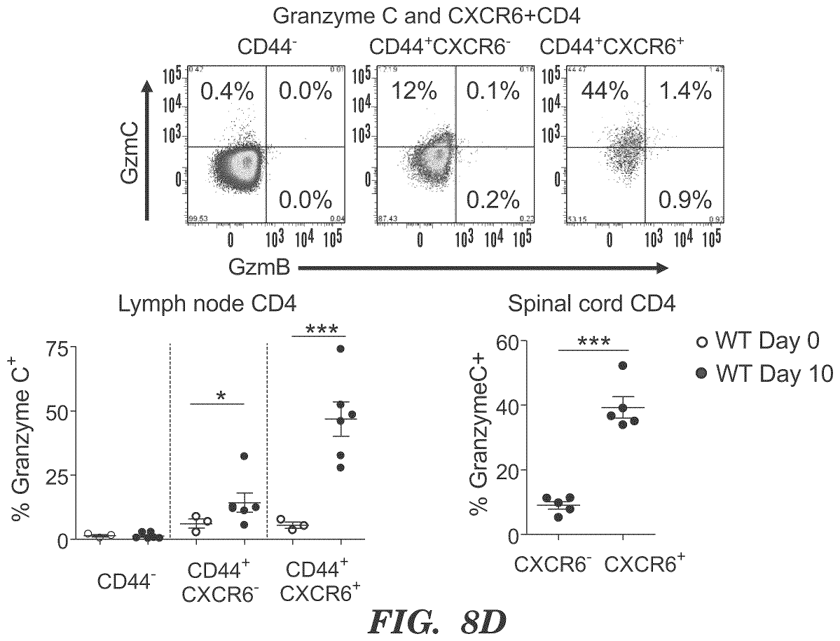

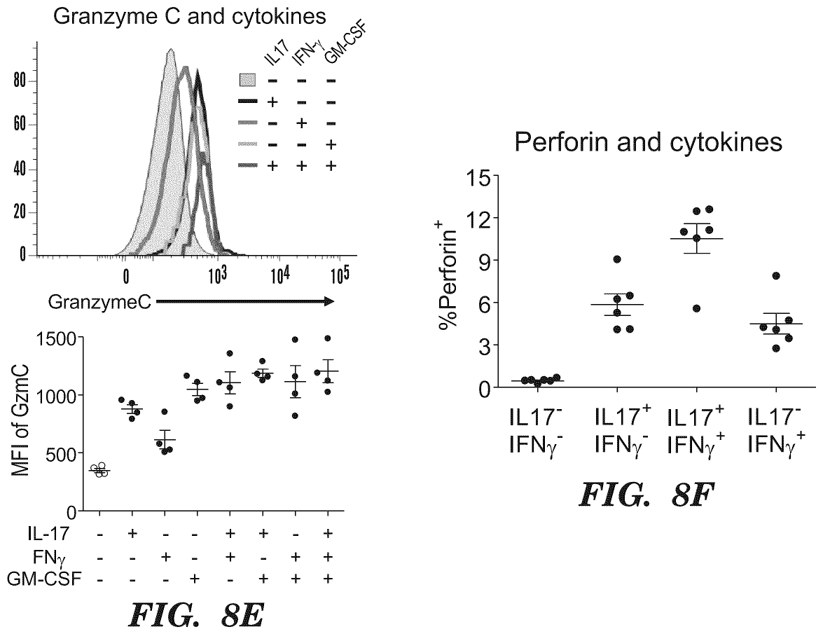

FIGS. 8A-8F present data that show the CXCR6-bearing subset of WT CD4 effector cells produces multiple cytokines and expresses Gzmc and Prf1 and highly express IL-1 and IL-23 receptors on the surface. (FIG. 8A) CXCR6 identifies the IL-17⁺, GM-CSF⁺ and IFNγ⁺ CD4 effector cells in EAE. (FIG. 8B) Gene expression of indicated CD4 cells of WT mice in EAE. (FIG. 8C) Activation markers and cytokine receptors expression on the surface of indicated CD4 cells in EAE. (FIG. 8D-F) Granzyme C and perforin are highly expressed in CXCR6⁺CD4 cells, especially in the cells producing two or more of the cytokines IL-17 and/or IFNγ and/or GM-CSF. Immunized wild-type mice were used to characterize the properties of CXCR6⁺CD4 cells.

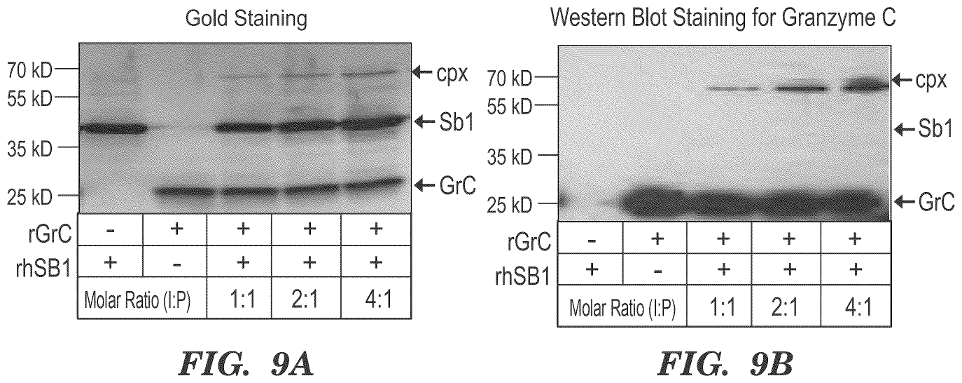

FIGS. 9A and 9B present data that show SerpinB1 inhibits Granzyme C. (FIG. 9A) Gold staining of protein showing formation of an inactive covalent higher molecular weight complex on incubation of pure SerpinB1 with pure Granzyme C. (FIG. 9B) Western blot analysis of Granzyme C in the covalent complex with SerpinB1. Formation of a covalent complex with target proteases is the inhibitory mechanism unique to Serpins.

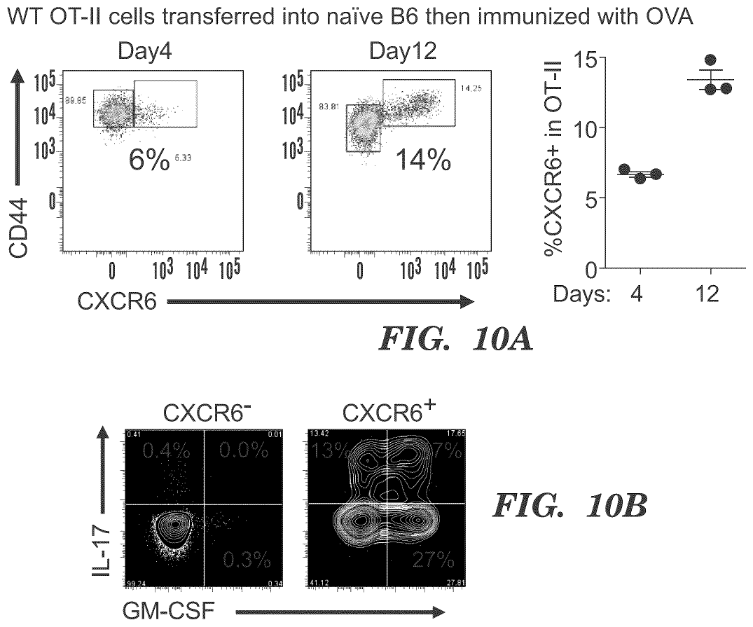

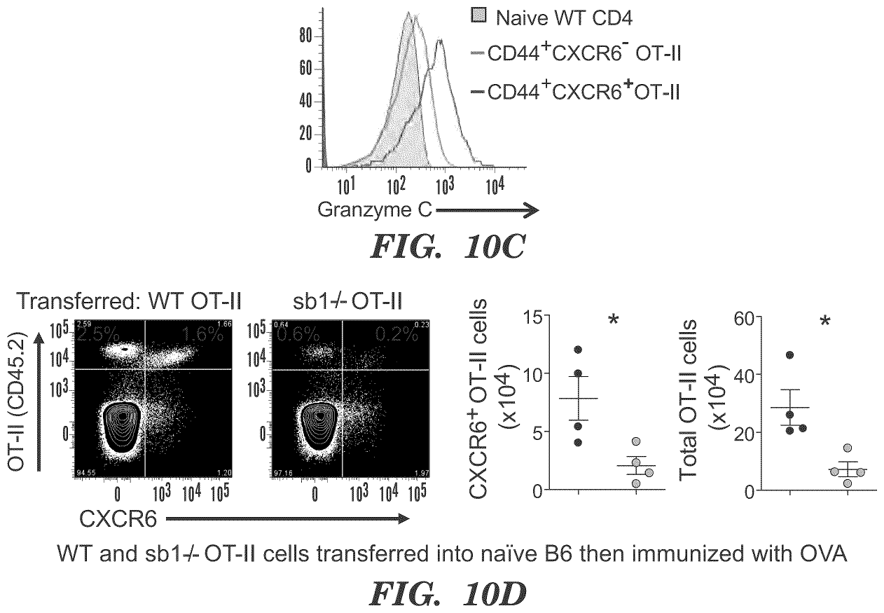

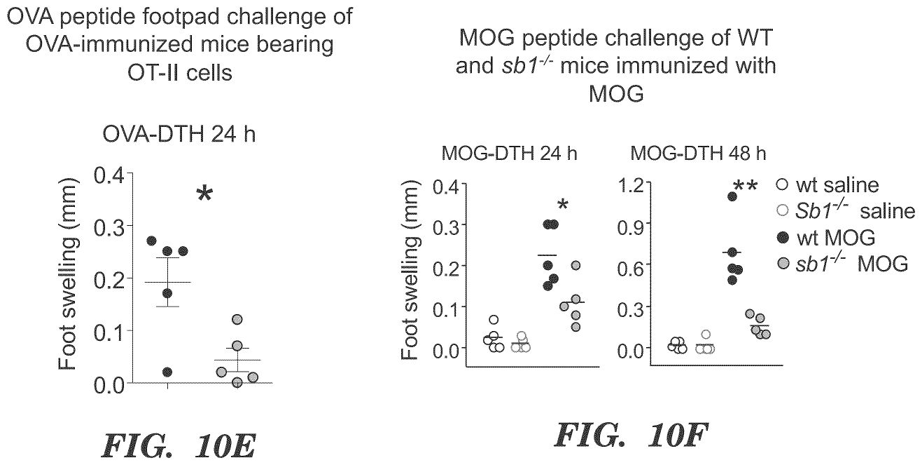

FIGS. 10A-10F present data that show CXCR6 also marks “delayed hypersensitivity” CD4 cells that are generated in response to antigen, produce multiple cytokines, require serpinB1 for expansion and for induction of footpad swelling on challenge with antigen. (FIG. 10A-10C) Naïve WT ovalbumin (OVA)-sensitive (OT-II) cells were transferred into naïve WT mice, then immunized with OVA peptide. (FIG. 10A) CXCR6⁺OT-II cells quantified on the indicated days. (FIG. 10B) CXCR6⁺OT-II cells produce multiple cytokines as in the EAE system. (FIG. 10C) CXCR6⁺OT-II cells highly express granzyme C as in the EAE system. (FIGS. 10D and 10E) WT and sb1^{-/-} OT-II cells were transferred into naïve WT mice and then immunized with OVA peptide. (FIG. 10D) On day 10, total OT-II cells and CXCR6⁺OT-II cells were quantified. (FIG. 10E) On day 7, indicated mice were challenged with OVA peptide in the footpad, and footpad swelling was quantified 24 hours later. (FIG. 10F) WT and sb1^{-/-} mice were immunized with MOG peptide by the method that induces EAE. On day 6, the mice were challenged with MOG peptide in the footpad, and footpad swelling was measured at indicated times. (FIG. 10D-10F) Red symbols indicate Sb1^{-/-}; black indicate WT.

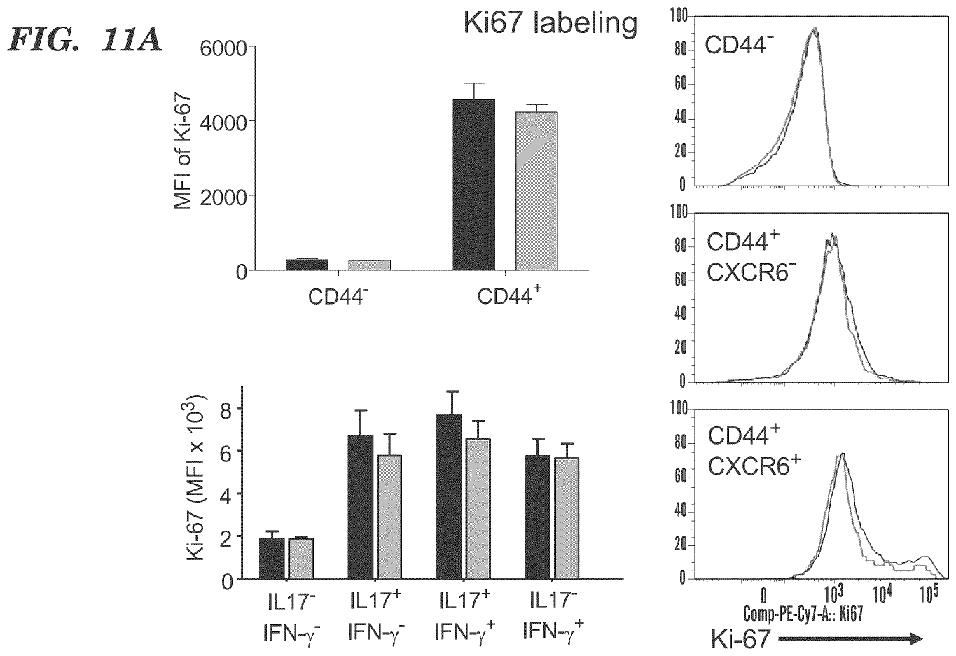

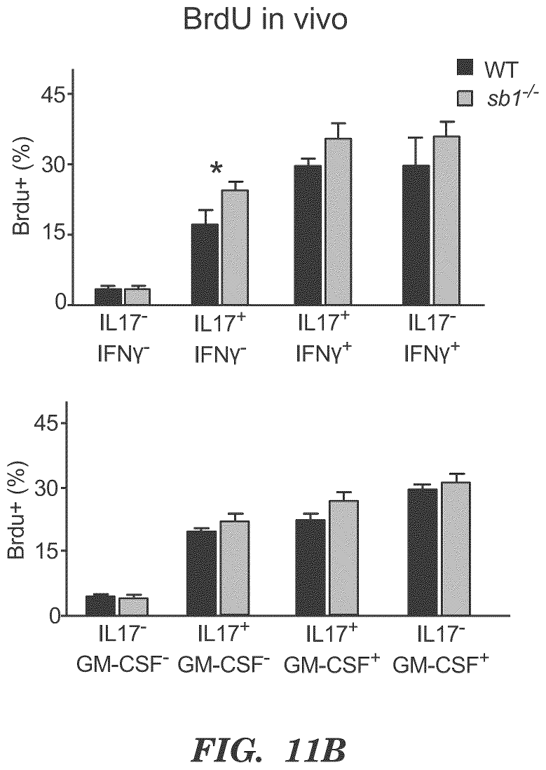

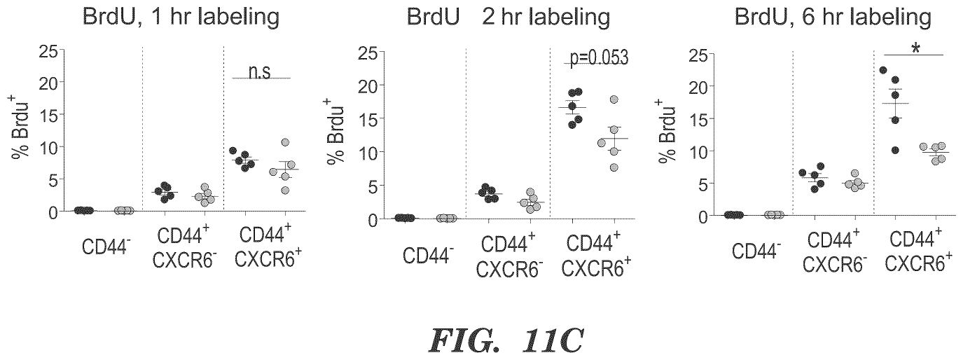

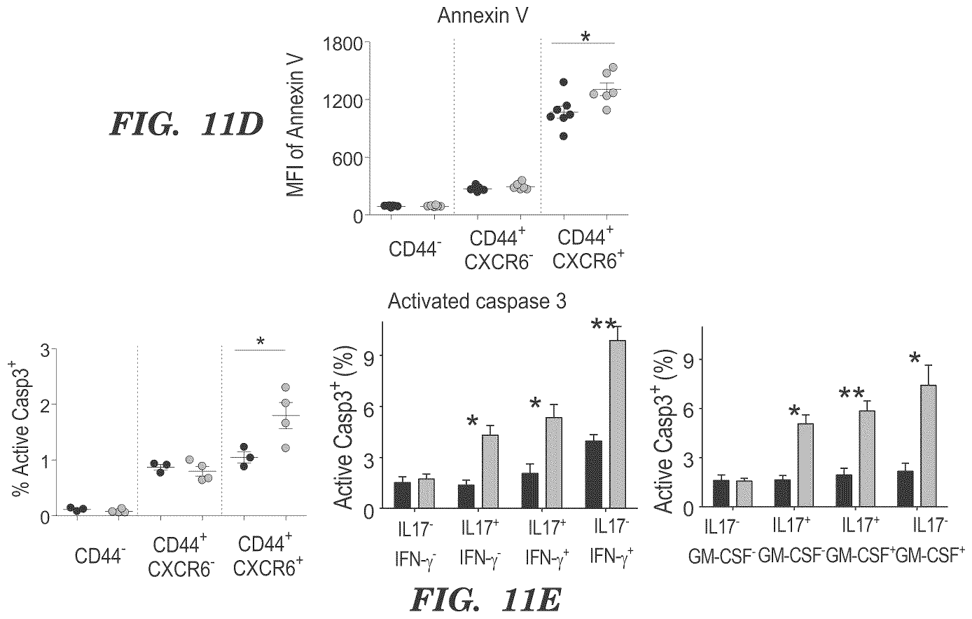

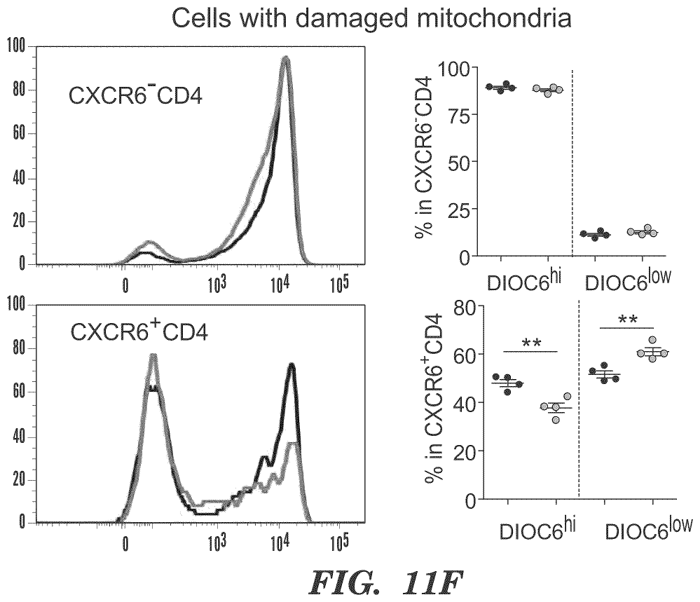

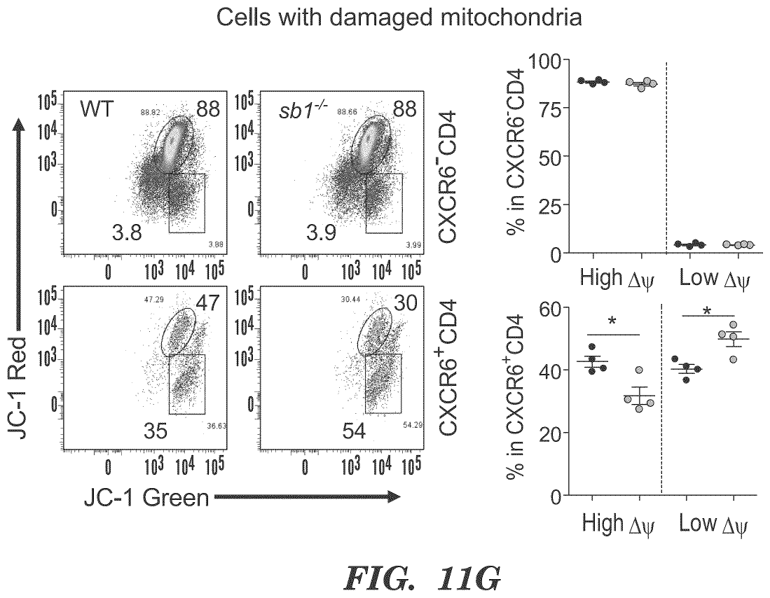

FIGS. 11A-11G present data that show markers of proliferation and markers of survival of CXCR6+ WT and Sb1^{-/-} CD4 effector cells in EAE. (FIG. 11A) Ki-67 labeling in indicated CD4 cell subsets. (FIG. 11B) Quantification of BrdU in vivo labeling of the indicated CD4 cells. (FIG. 11C) Dynamic analysis of BrdU incorporation in CXCR6⁺CD4 cells. Proliferation of CXCR6⁺CD4 cells is robust and is not different between WT and sb1^{-/-} cells. But the sb1^{-/-}CXCR6⁺CD4 cells have increased cell death. (FIG. 11D) Quantitation of annexin V staining of indicated CD4 cells. (FIG. 11E) Quantification of indicated cells expressing active-Caspase 3 (FIGS. 11F and 11G) Quantification of cells with damaged mitochondria in indicated mice. Sb1 is not required for proliferation of CXCR6⁺ CD4 effector cells. However, cell death of CXCR6⁺ CD4 effector cells is increased in sb1^{-/-} mice. Red symbols indicate Sb1^{-/-}; black indicate WT.

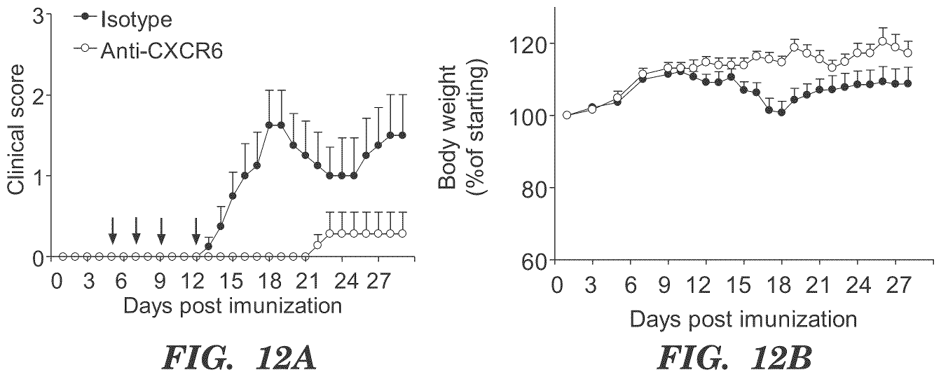

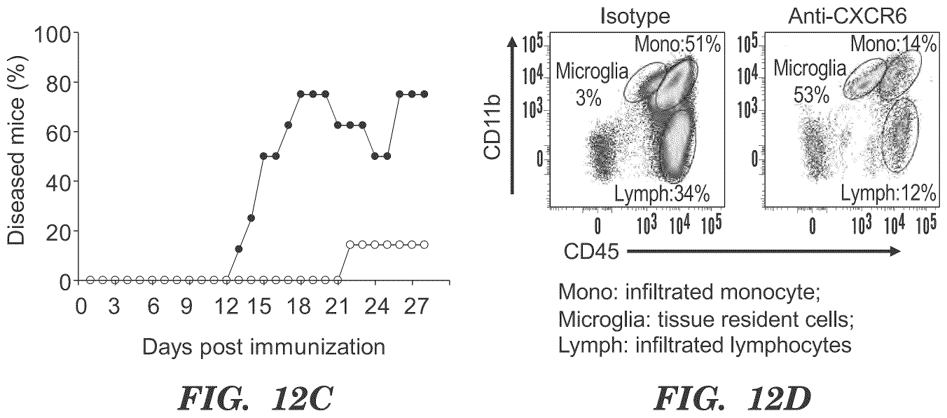

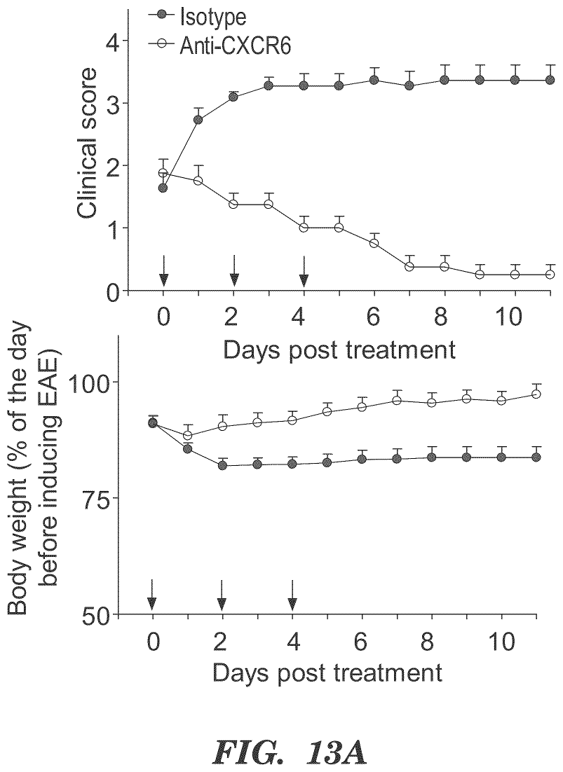

FIGS. 12A-12D present data that show anti-CXCR6 antibody treatment prevents EAE. WT mice were induced for EAE, and then treated with isotype control antibody (8 mice) or anti-mouse CXCR6 antibody (7 mice) (300 μg/mouse/injection) at days 5, 7, 9 and 12. (FIG. 12A) Mean clinical score. (FIG. 12B) Mean body weights. (FIG. 12C) Frequency of diseased mice. (FIG. 12D) Infiltrated lymphocytes and myeloid cells in the spinal cord on day 27.

FIGS. 13A and 13B present data that show treatment with anti-CXCR6 antibody is effective as a treatment for EAE. (FIG. 13A) WT mice were induced for EAE. Treatment with isotype control antibody (400 μg/treatment) (11 mice) or anti-CXCR6 antibody (8 mice) was initiated for individual mice on the day that disease was first detected (days 11-15; initial scores 1-3). Subsequent treatments were administered 2 and 4 days later (arrows). (FIG. 13A, top panel) Mean clinical score. (FIG. 13A, bottom panel) Body weight. (FIG. 13B) Six WT mice were induced for EAE, and three mice each were treated on day 10 with 400 μg isotype control or anti-CXCR6 antibody; the mice were sacrificed on day 11. (FIG. 13B, top-panel) Representative flow cytometry measuring cytokines IL-17 and GM-CSF production by lymph node CD4 cells. (FIG. 13B, bottom panel). Cumulative results for production of these cytokines. The decrease of cytokine-producing cells indicates that the anti-CXCR6 antibody treatment depletes the cytokine-producing CXCR6⁺ pathogenic CD4 cells.

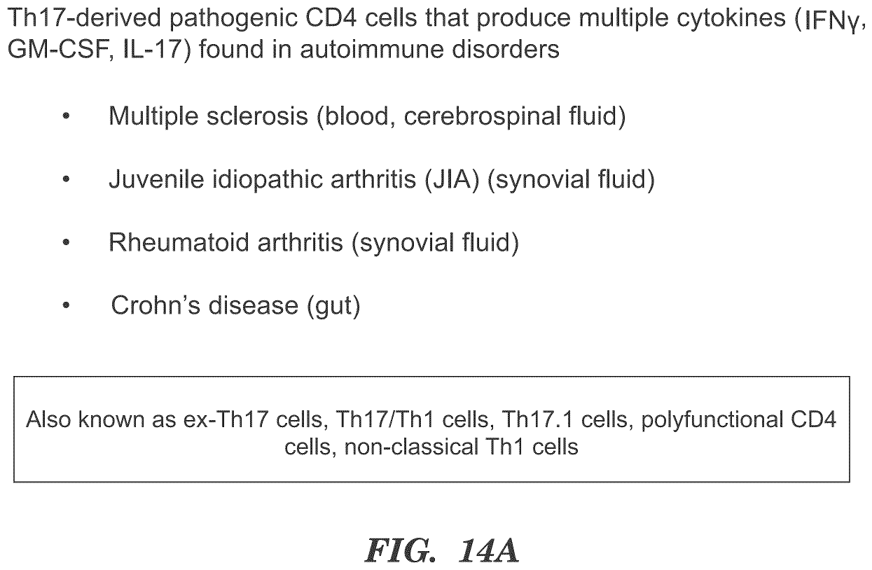

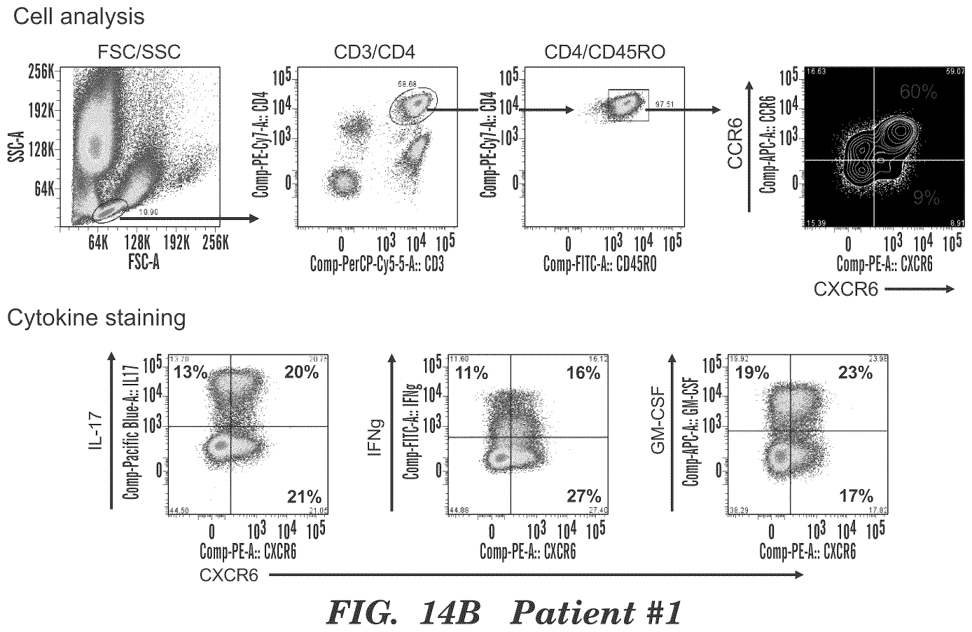

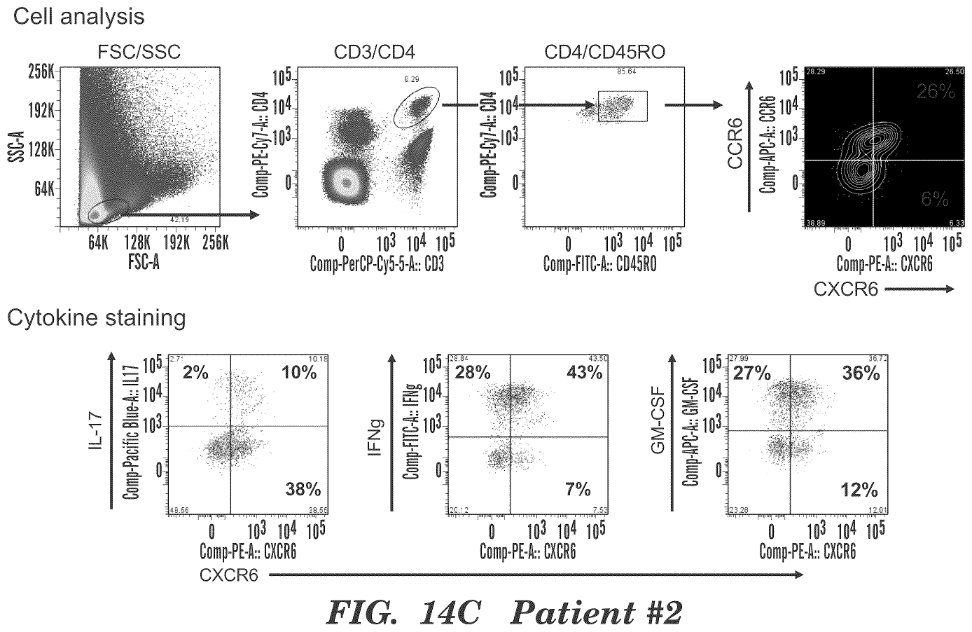

FIGS. 14A-14C present data that show human CXCR6+ CD4 cells are present in inflammatory synovial fluids of patients with rheumatoid arthritis and these cells produce multiple cytokines as in the murine EAE system. (FIG. 14A) Background information on pathogenic CD4 cells in autoimmune disorders. (FIG. 14B, C). Analysis of synovial fluid cells of two patients with rheumatoid arthritis including frequency of CXCR6+ CD4 cells and their production of IL-17, IFNγ and GM-CSF.

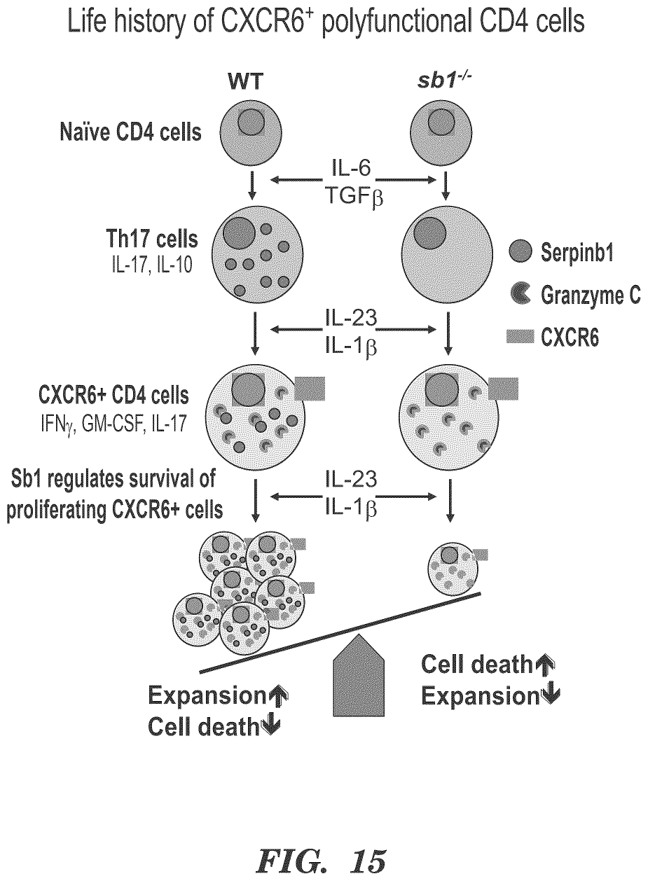

FIG. 15 presents a schematic that shows the generation of CXCR6+ cells based on the EAE data. The regulatory step is indicated in which sb1 prevents cell death of robustly proliferating CXCR6+ cells by inhibiting a protease, which may be the human equivalent of murine granzyme C, and thereby determines the size of the resulting population of pathogenic CXCR6+ CD4 cells.

### Example 3: Serpinb1 Controls Encephalitogenic TH Cells in Neuroinflammation

SerpinB1, a protease inhibitor and neutrophil survival factor, was recently linked with IL-17-expressing T cells. Here, the results show that serpinB1 (Sb1) is dramatically induced in a subset of effector CD4 cells in experimental autoimmune encephalomyelitis (EAE). Despite normal T cell priming, Sb1−/− mice are resistant to EAE with a paucity of TH cells that produce two or more of the cytokines, IFNγ, GM-CSF and IL-17. These multiple cytokine-producing CD4 cells proliferate extremely rapidly, highly express the cytolytic granule proteins perforin-A, granzyme A (GzmA) and GzmC, and surface receptors IL-23R, IL-7Rα and IL-1R1, and can be identified by the surface marker CXCR6. In Sb1−/− mice, CXCR6+ TH cells are generated but fail to expand due to enhanced granule protease-mediated mitochondrial damage leading to suicidal cell death. Finally, anti-CXCR6 antibody treatment, like Sb1 deletion, dramatically reverts EAE, strongly indicating that the CXCR6+ T cells are the drivers of encephalitis.

### INTRODUCTION

Multiple sclerosis and murine experimental autoimmune encephalomyelitis (EAE) are chronic demyelinating disorders of the central nervous system driven by self-reactive TH cells (1) The disease-inducing autoimmune T cells, which are present at low numbers in the periphery and as expanded populations in the CNS, were initially thought to be TH1-cells because disease is abrogated by deletion of the IL-12 subunit p40 (2, 3). With the discoveries that p40 is also a subunit of IL-23 and IL-23 plays a pivotal role in mediating disease (4-7), MS was re-interpreted as TH17-driven (8, 9). More recent studies established that TH17 cells themselves are not pathogenic, but are converted in vivo under the priming of myeloid cell-derived IL-1β and IL-23 into pathogenic (encephalitogenic) TH cells, the true drivers of disease (4, 10-14). These cells produce IFNγ and GM-CSF (10, 15-18), the latter required for encephalitogenicity (16-18). Despite the importance of the encephalitogenic TH cells, little else is known about their nature or the factors and pathway that drive their development.

In cytolytic CD8 cells and NK cells, powerful granule serine proteases that are regulated by endogenous inhibitors called serpins play pivotal roles in immune surveillance against tumors and viral infection, while simultaneously maintaining immune homeostasis (19-26). Whether analogous granzyme-serpin regulation also exists in CD4 cells is not known. SerpinB1, previously called MNEI (monocyte/neutrophil elastase inhibitor), is an ancestral member of the superfamily of serpins (SERine Protease Inhibitors). It is a highly efficient inhibitor of elastinolytic and chymotryptic proteases that has been best studied in neutrophils (27-31). For example, in bacterial lung infection, serpinB1 protects against inflammatory tissue injury and neutrophil death, and in naïve mice, serpinB1 preserves the bone marrow reserve of mature neutrophils by restricting spontaneous cell death mediated by the granule serine proteases cathepsin G and proteinase-3 (32-35). Recently, it has been demonstrated that serpinB1 selectively restricts expansion of IL-17-expressing γδ T cells (36) and NK T cells (37), findings that led us to study adaptive Th cell development where Sb1 was identified as a signature gene of Th17 cells (38).

Provided herein are surprising results that Sb1 expression is required for development of paralysis in MOG-immunized mice. A highly selective subset of IFNγ- and GM-CSF expressing IL17+ serpinB1-dependent CD4 cells are identified herein in the periphery of immunized mice at onset of disease. The isolation of these serpinB1-dependent primed T cells and their molecular and functional signatures and developmental pathway in EAE are shown. The results demonstrate that these are the T helper cells responsible for disease.

### RESULTS

### SerpinB1 is Highly Expressed in TH Cells in EAE.

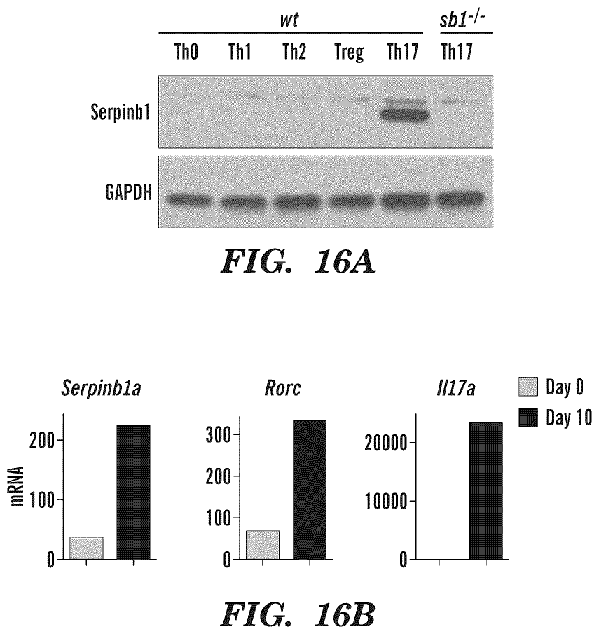

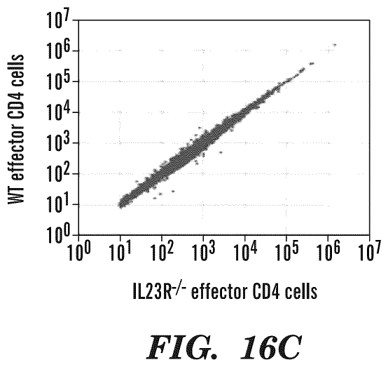

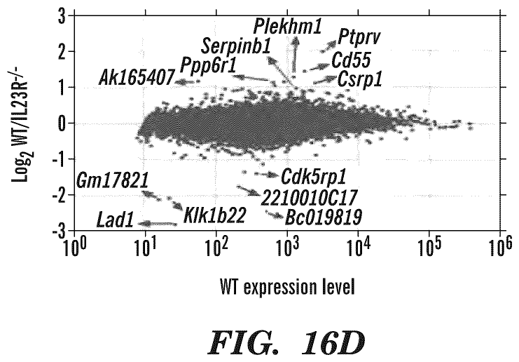

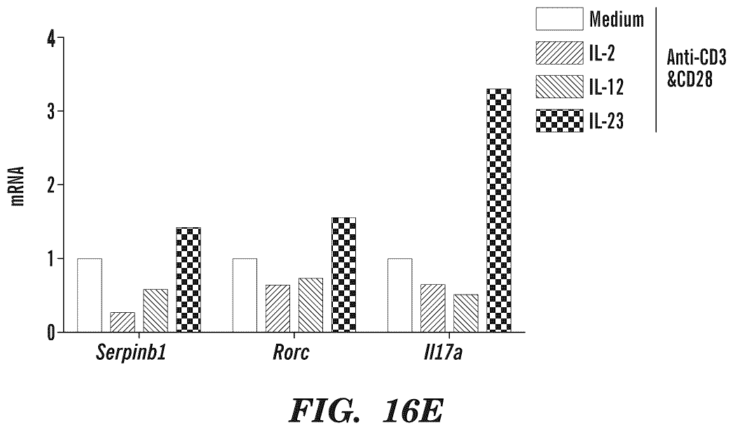

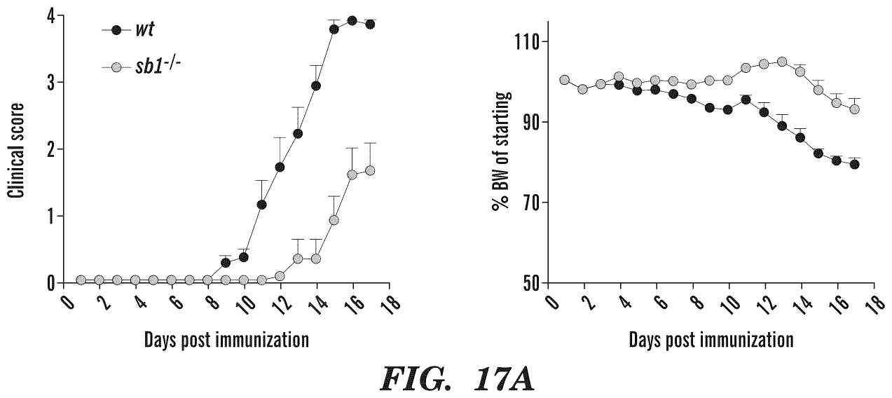

Previously, Sb1 was identified as being preferentially expressed in TH17 cells by studying 129S6 strain mouse cells in vitro. In preparation for working with the EAE model, naïve CD4 cells of C57Bl/6 mice were polarized and confirmed to have select expression of Sb1 in TH17 cells driven by IL-6 and TGF3, consistent with previous findings (38) (FIG. 16A). It was then determined that Sb1 is dramatically upregulated in vivo along with Rorc and Il17a in effector CD4 cells during EAE development (FIG. 16B). To date, the factors controlling expression of Sb1 in TH cells are unknown. Online gene arrays for mice deleted for the Th17 inducer serine/threonine protein kinase (Sgk)-1 (39) revealed that Sb1 is among the top downregulated genes in IL23-stimulated Sgk1-deficient TH17 cells, suggesting a correlation between Il23r and Sb1 in TH17 cells. To investigate this putative link, mice were generated with Il23r deleted in CD4 cells (Il23rΔCD4) by crossing Il23rfl/fl mice with CD4-Cre mice. The transcriptome of wt and Il23rΔCD4 effector (CD44hiCD62Llo) CD4 cells from lymph node of mixed chimeric mice at onset of EAE was compared. Surprisingly, wt and Il23rΔCD4 effector CD4 cells were not very different at the transcriptome level (FIG. 16C), and only a dozen genes were increased more than 2 fold in wt cells (FIG. 16D). Among the prominent genes with skewed expression and known immune function, Sb1 was discovered, confirming the critical and direct role of IL-23R signals in inducing or maintaining Sb1 expression in effector CD4 cells. To further investigate the correlation between Il23r and Sb1 in TH17 cells, the IL-6/TGF3 in vitro TH17 cell differentiation system was investigated. Whereas adding IL-13 and/or IL-23 did not further increase Sb1 expression (data not shown), on restimulation, the in vitro generated TH17 cells need IL-23R signaling to maintain expression of Sb1, Rorc and 1117 (FIG. 16E).

### EAE Amelioration Due to CD4 Cel-Autonomous Deficiency of Sb1

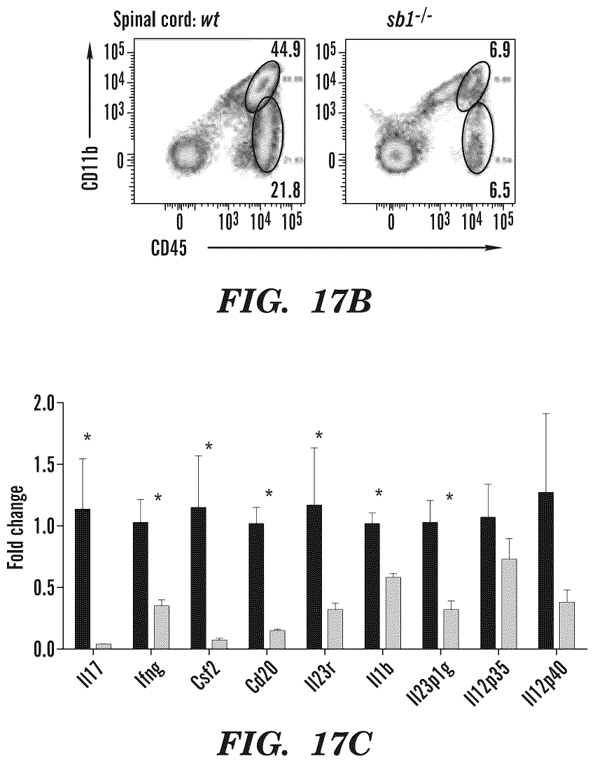

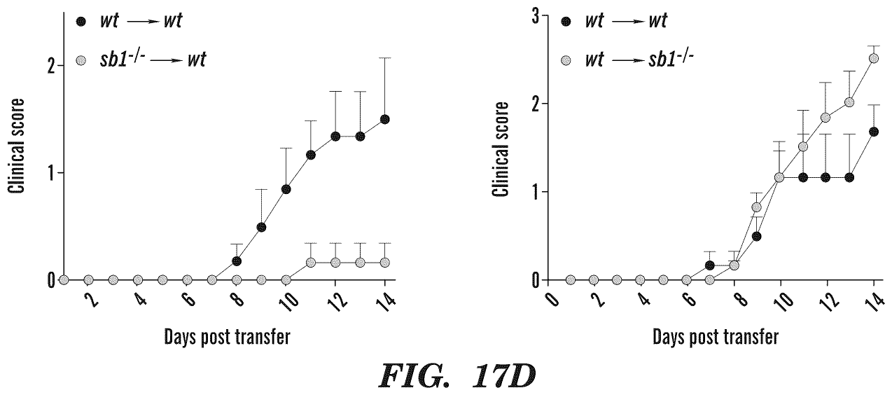

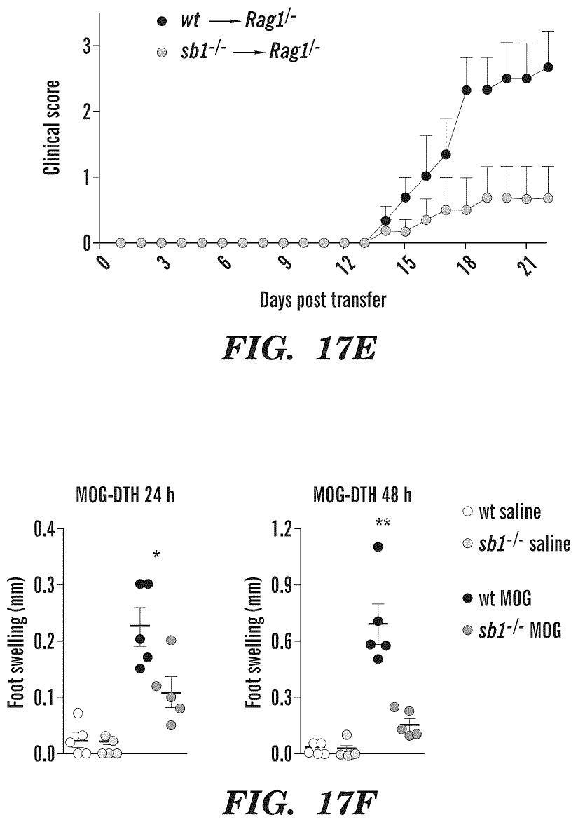

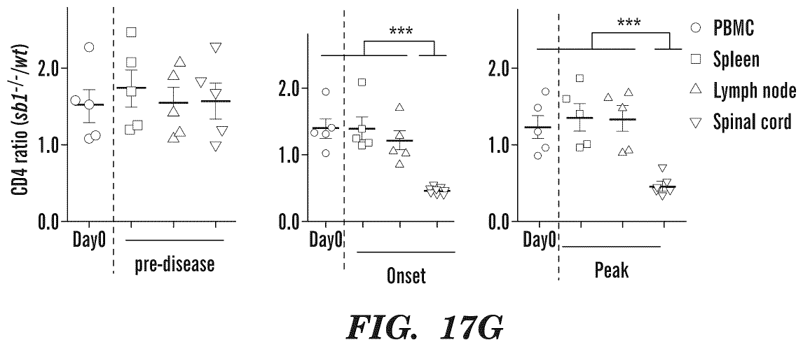

To determine whether the expression of Sb1 affects the encephalitogenic TH cells, EAE was induced in Sb1-gene-deleted mice. Compared to the severe encephalomyelitis that developed in MOG-immunized wt mice, Sb1−/− mice exhibited delayed and ameliorated disease (FIG. 17A). Fewer leukocytes, both lymphocytes and myeloid cells, infiltrated the spinal cord (FIG. 17B). The deficit of cells was reflected at the mRNA level in the decrease of TH- and myeloid cell cytokines (FIG. 17C). Because Sb1 is expressed in multiple cells and very prominently in myeloid cells, adoptive transfer studies were performed to determine the T cell intrinsic properties of Sb1. It was discovered that, compared to wt T cells, Sb1−/− T cells of immunized mice were less encephalitogenic. Moreover, disease was not ameliorated in the reciprocal experiment (FIG. 17D), solidifying the notion that Sb1 influences the pathogenic potential of T cells. In a complementary model, naïve CD4 cells were transferred into Rag1/− mice prior to immunization. Clinical disease was attenuated in mice receiving Sb1−/− CD4 cells compared to mice receiving wt CD4 cells, and immune cell accumulation in spinal cord was blunted (FIG. 17E). Further, comparison of MOG-specific delayed-type hypersensitivity (DTH) responses of MOG-immunized wt and Sb1−/− mice showed that T cell priming was already impaired in the periphery in Sb1−/− mice (FIG. 17F). Finally, a mixed chimeric mouse model revealed that the ratio of Sb1−/− to wt CD4 cells in the periphery did not change following MOG immunization, but the Sb1−/− to wt CD4 cell ratio decreased in the spinal cord (FIG. 17G), a phenotype that largely replicates that of wt:Il23r-deficient mixed chimeric mice (11). The cumulative findings indicate that attenuation of encephalomyelitis and paucity of immune cells in the spinal cord of Sb1−/− mice are due to Sb1 absence in CD4 cells.

### Sb1 Controls IFNγ+ and GM-CSF+CD4 Cells During Priming

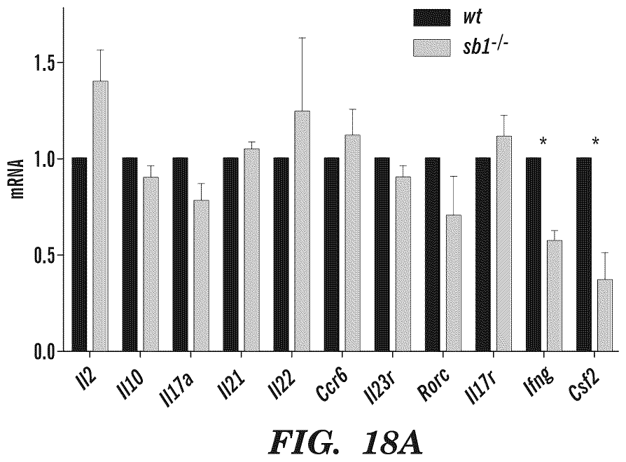

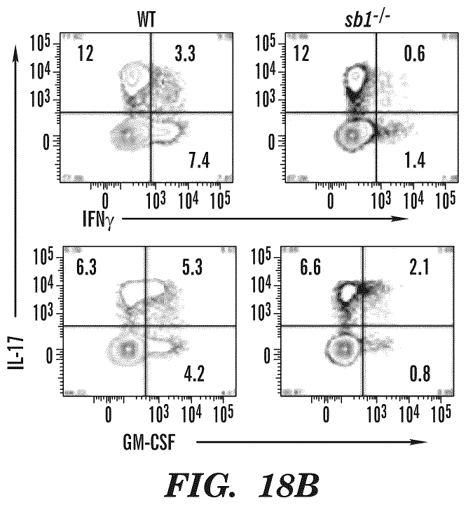

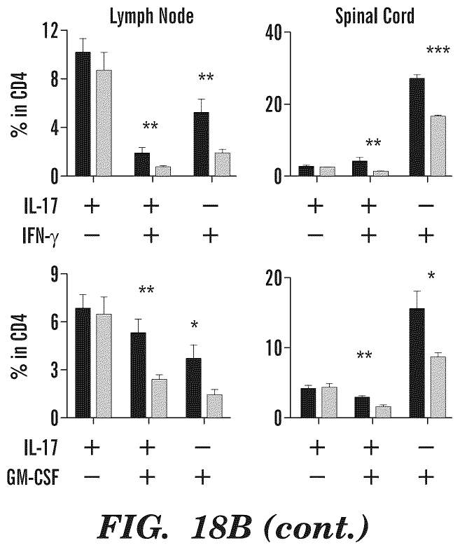

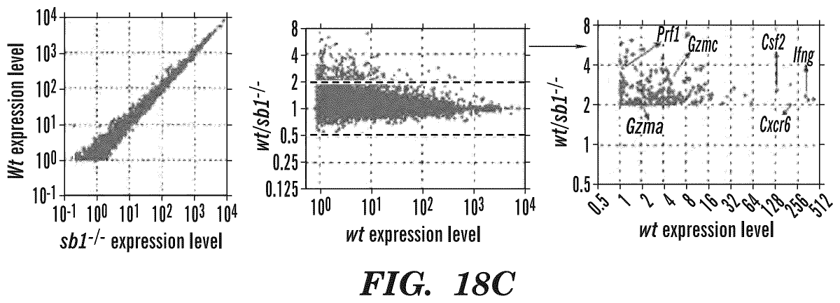

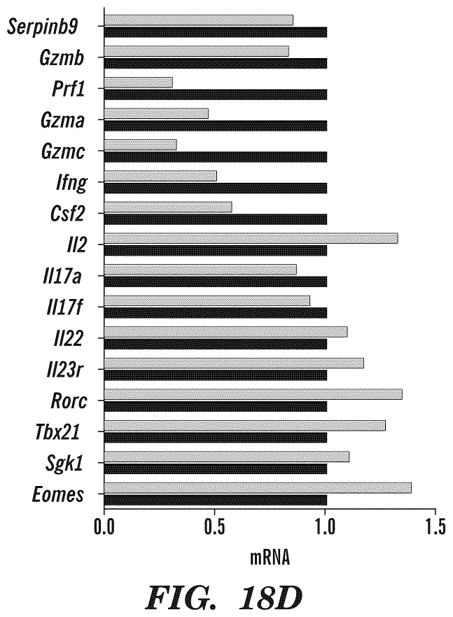

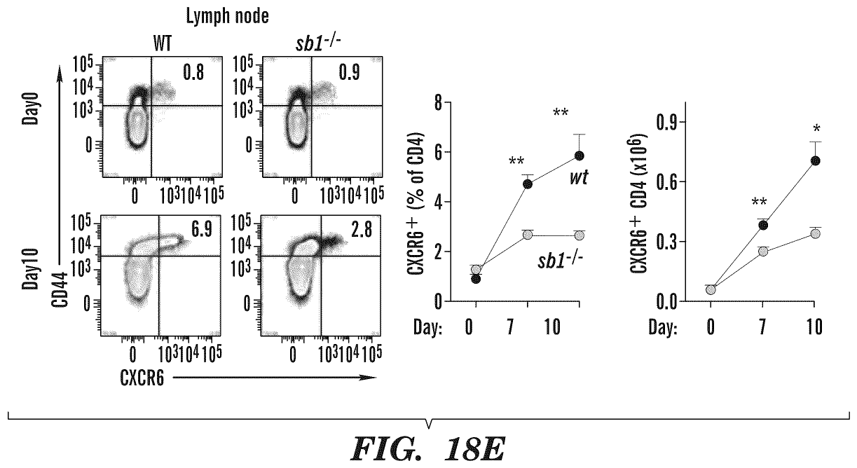

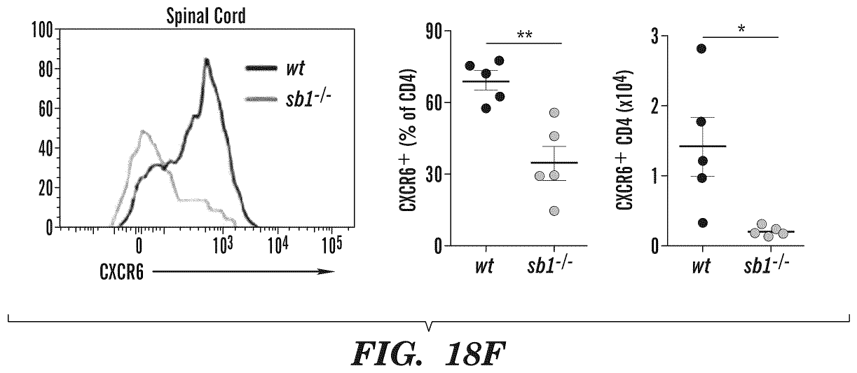

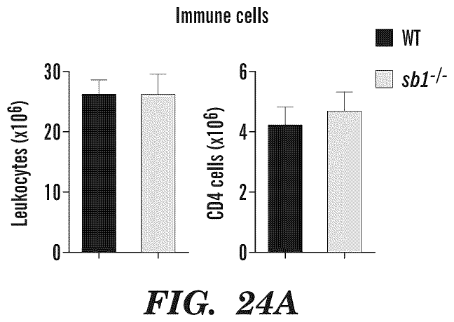

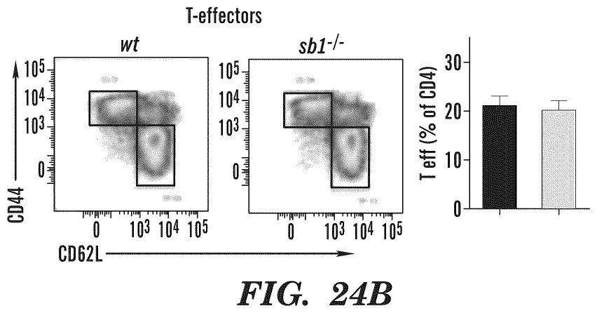

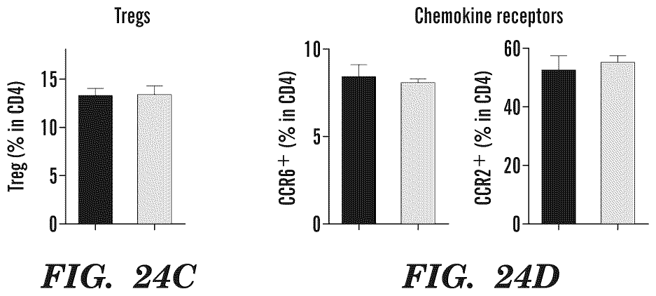

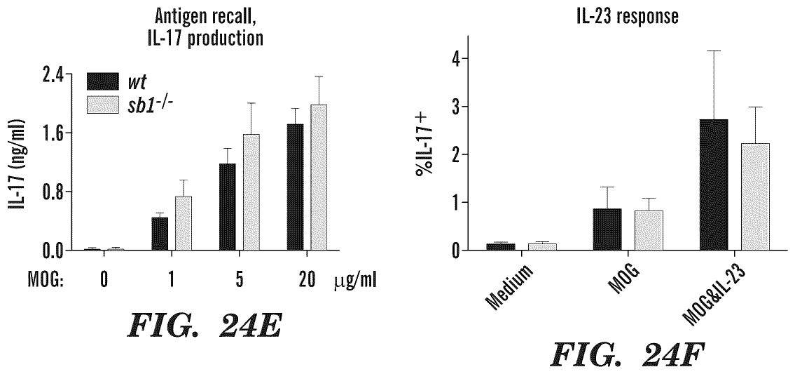

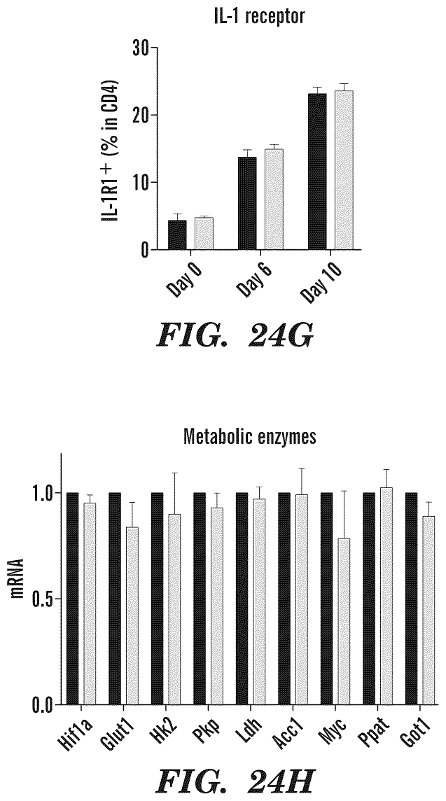

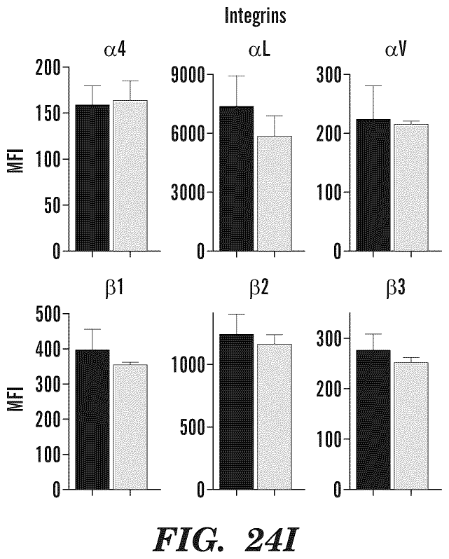

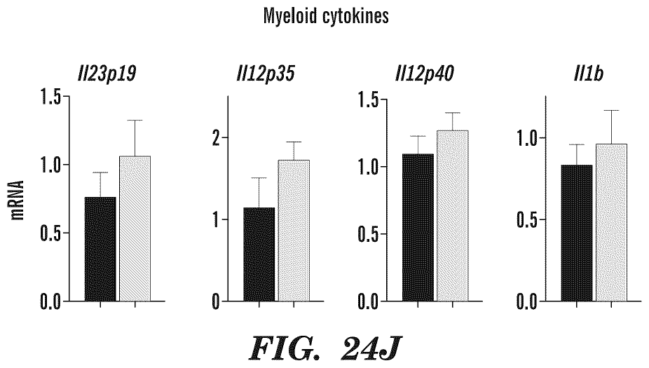

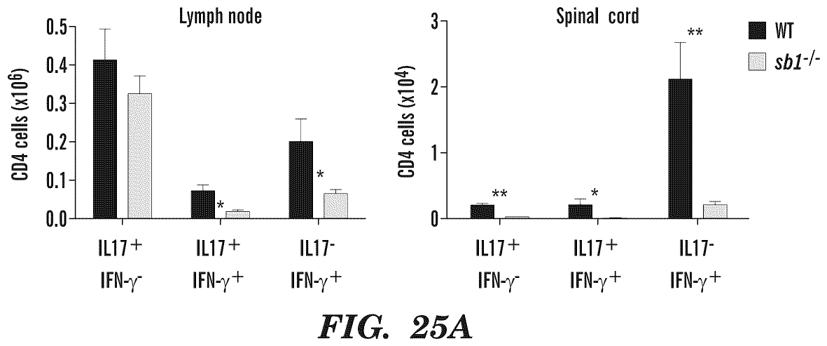

To determine what causes the deficit of spinal cord T cells, draining LN CD4 cells at disease onset were examined. No differences were found between Sb1−/− and wt mice in immune cell counts or frequencies of effector (CD44+) CD4 T cells, T-regulatory cells or CCR6 and CCR2 expressing CD4 cells (FIG. 24A-D). There were also no differences between the genotypes in recall properties, IL-17 production, responsiveness to IL-23, upregulation of IL-1R1, TH17 metabolic enzymes, expression of integrins including VLA4 and LFA1 and expression of myeloid cytokines (FIG. 24E-J). Moreover, IL-23R and many other genes generally associated with TH17 cells are expressed at normal levels in Sb1−/− effector CD4 cells (FIG. 18A). However, expression of Csf2 and Ifng, encoding GM-CSF and IFNγ, respectively, was decreased in lymph node of Sb1−/−effector CD4 cells compared to corresponding wt cells (FIG. 18A).

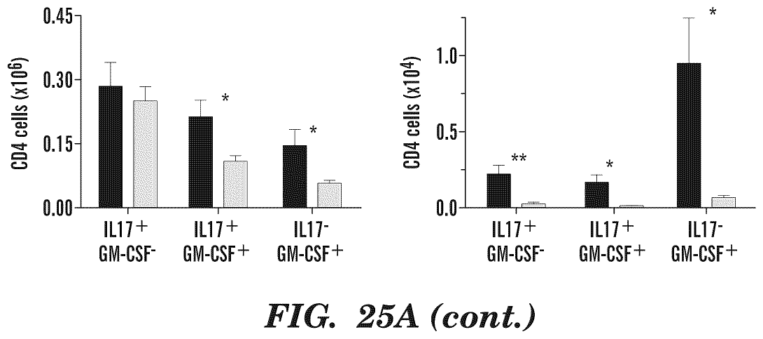

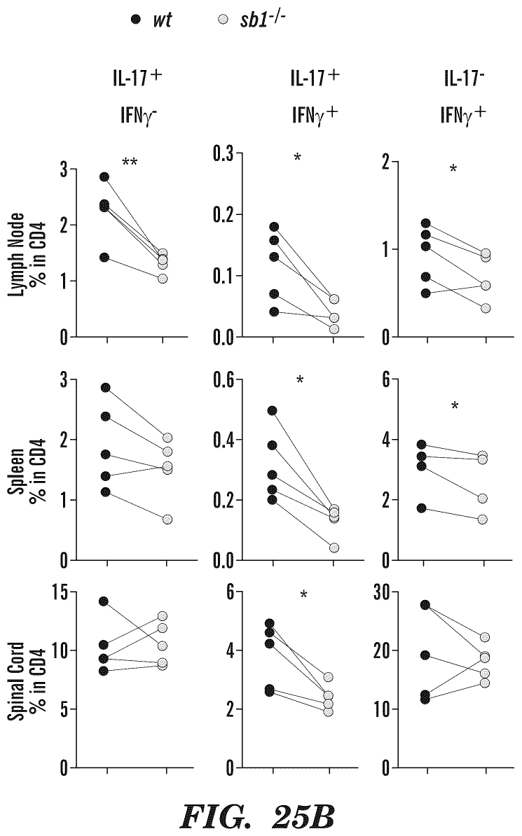

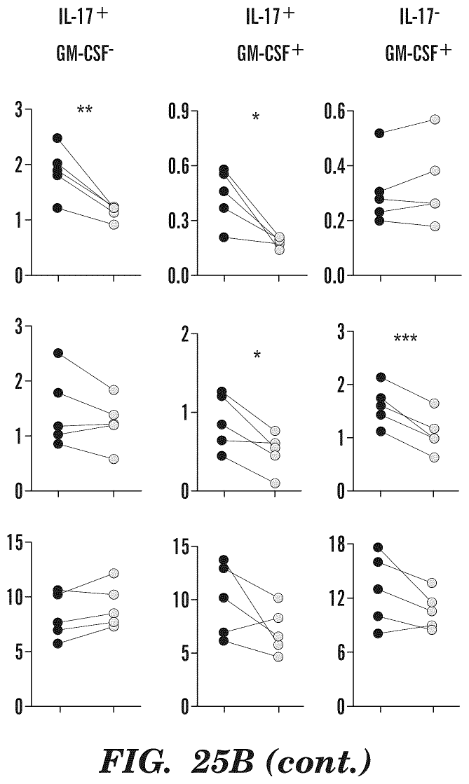

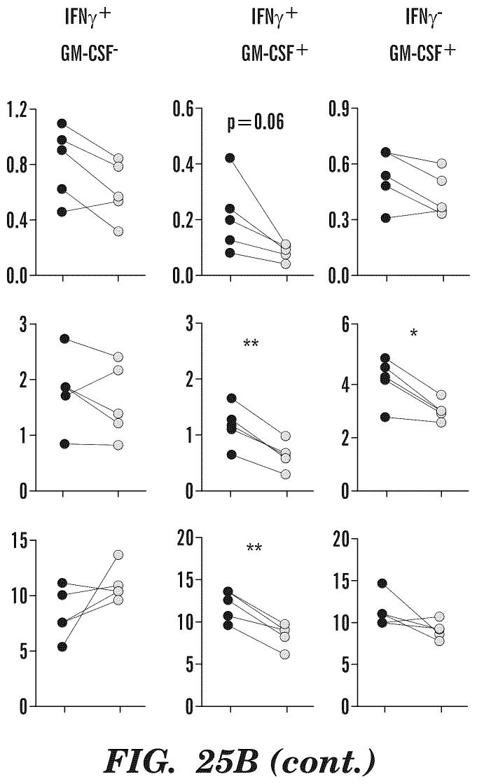

To determine whether the decreased expression of Csf2 and Ifng represents decreased cytokine per cell or fewer cytokine-expressing cells, lymph node and spinal cord CD4 cells were examined by flow cytometry. The frequencies of IL-17 single positive (SP) cells were not different in Sb1−/− and wt mice; however, the frequencies of cytokine double positive (DP) (IL17+IFNγ+, IL17+GM-CSF+) cells as well as GM-CSFγ SP, and IFNγ SP cells were decreased in lymph nodes and spinal cord of Sb1−/− mice (FIG. 18B). TH cells that produce multiple cytokines have been previously described in affected organs of patients with autoimmunity (40, 41), and GM-CSF+ cells are known to be essential for autoimmune neural inflammation (16-18). In the lymph node of wt and sb1−/− mice, the absolute numbers of cytokine-producing cells reflected the frequency patterns, but in the spinal cord, the absolute numbers of all Sb1−/− CD4 cells were greatly decreased (FIG. 25A). In MOG-immunized mixed bone marrow chimeras, frequencies of most cytokine double positive (DP) Sb1−/− CD4 cells were skewed downward (FIG. 25B). Cumulatively, the findings support the concept that encephalitogenic TH cells, identifiable by production of GM-CSF and IFNγ, are expanded already in the lymph node of MOG-immunized mice of both genotypes, but their frequency is decreased in Sb1−/− mice.

Signature Genes Identified for serpinB1-Dependent TH Cells in EAE

Next, genes were identified that confer encephalitogenic properties to TH cells through serpinB1. The transcriptome of Sb1−/− and wt effector CD4 cells isolated from LN at disease onset was analyzed, anticipating that other encephalitogenity-conferring genes would be decreased along with Csf2 and Ifng among Sb1−/− effector cells. Of 9,650 expressed genes, 258 genes were decreased >2-fold in Sb1−/− compared with wt cells, and no genes were increased >2 fold (FIG. 18C). From among the decreased genes, a subset were selected with immune-related functions for further study. Those verified by qRT-PCR are Ifng and Csf2, as expected, and also Gzmc (GzmC), Gzma (GzmA) and Prf1 (perforin A), which are components of cytotoxic granules (FIG. 18D). Of note, cathepsin L, which promotes differentiation of TH-17 cells and is inhibited by serpinB1 (38) was not among the genes underexpressed in Sb1−/− effector CD4 cells. The skewed genes included the chemokine receptor Cxcr6 encoding CXCR6 (42), which was verified by flow cytometry.

CXCR6 Marks serpinB1-Dependent Encephalitogenic TH Cells

Next, the CD4 cells expressing CXCR6 were examined as a function of time during EAE development in wt mice. CXCR6+ CD4 cells, which comprised <1% in naïve mice, increased after MOG immunization to ~6% in the lymph node (FIG. 18E) and constituted the major population (~70%) in the spinal cord at peak of disease (FIG. 18F). CXCR6+CD4 cells also increased in frequency in LN of Sb1−/− mice post-immunization, but not to the same extent as in wt mice, and failed to accumulate in the Sb1−/− spinal cord. The difference between the genotypes can be best appreciated by comparing the absolute cell numbers (FIGS. 18E, 18F right panels).

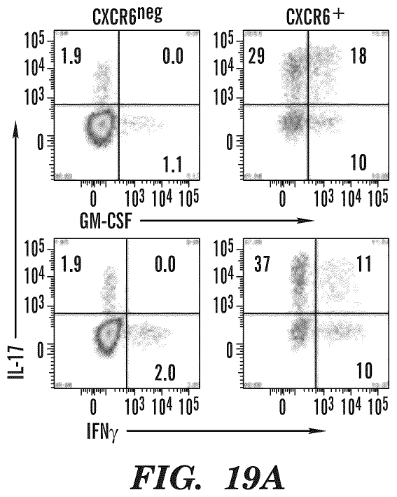

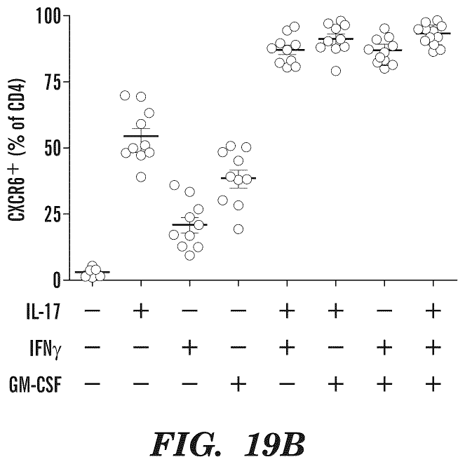

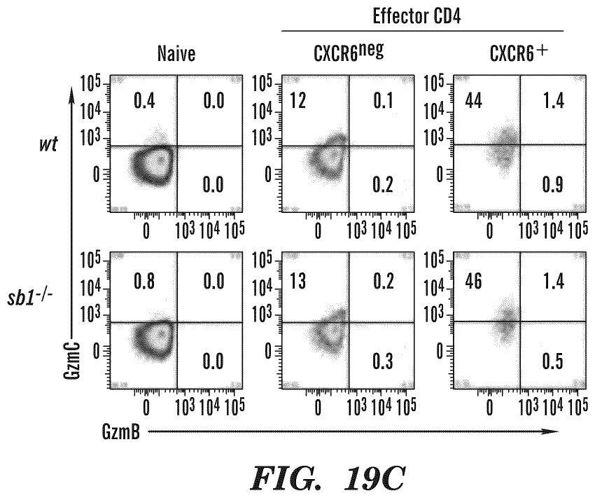

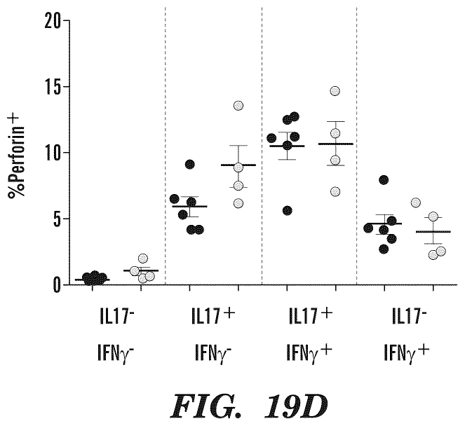

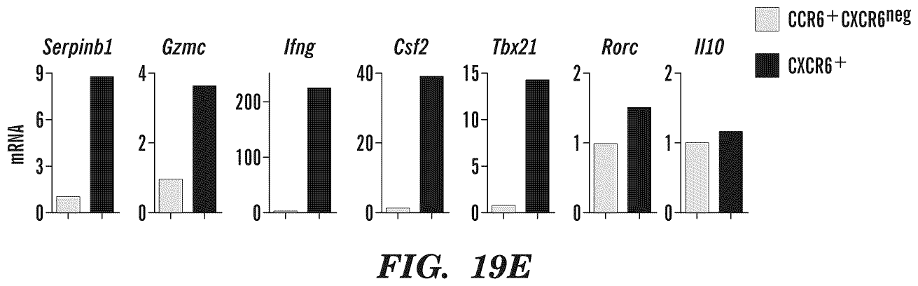

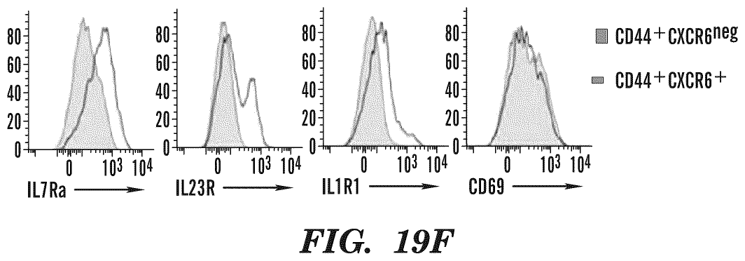

Combined analysis of CXCR6 and cytokines showed that essentially all LN CD4 cells that produce two or more of the cytokines IL-17, GM-CSF, and IFNγ were CXCR6+, as were half of IL-17 SP, a third of GM-CSF-SP, and a smaller fraction of IFNγ-SP cells (FIGS. 19A,19B). Strikingly, GzmC, but not GzmB, was preferentially expressed in CXCR6+CD4 cells (FIG. 19C). Concomitantly, perforin-A expression, which was negligible in cytokine neg CD4 cells, was increased in IL-17 SP cells and further increased in IL17/IFNγ DP cells (FIG. 19D). It is likely that the granzymes and perforin-A are components of functioning cytotoxic granules as indicated by the increased surface expression of the granule membrane protein LAMP-1 (CD107a) after ex vivo stimulation (data not shown). On a ‘per cell’ basis, the content of GzmC and perforin were not different between the genotypes. Except for Csf2 and Ifng, the signature genes Gzmc, Gzma, Prf1 and Cxcr6 identified here for in vivo generated encephalitogenic TH cells differ from the signature genes of pathogenic TH17 cells generated in vitro (43). Compared with conventional TH17 cells (CCR6+CXCR6neg), CXCR6+CD4 cells in EAE showed increased Sb1, Gzmc, Tbx21, Csf2, and Ifng expression but comparable levels of Rorc and Il10 (FIG. 19E). Compared with CXCR6neg effector CD4 cells, CXCR6+CD4 cells had increased surface expression of IL7Ra, IL23R and IL1R1, but not PD-1, ICOS, CD69 and CD25 (FIG. 19F and data not shown). These findings strongly suggest that the CXCR6+ serpinB1-dependent CD4 cells are the TH17-derived encephalitogenic TH cells in EAE.

### Verifying the Function of CXCR6+ CD4 Cells in EAE

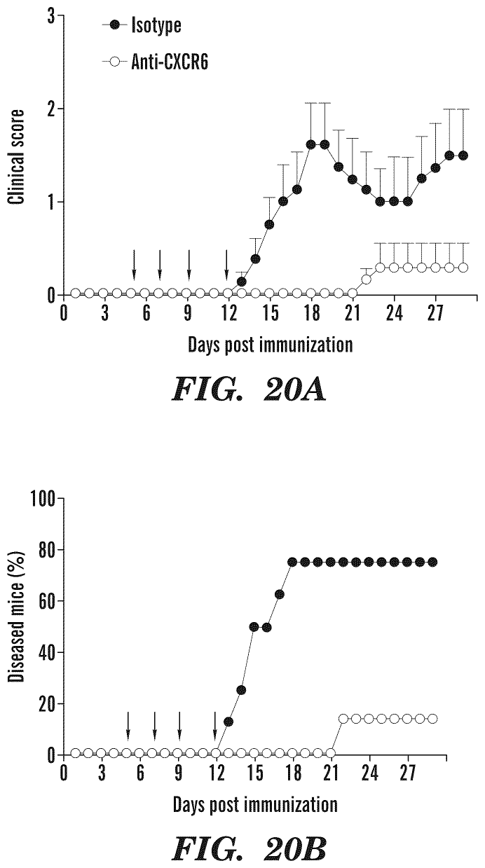

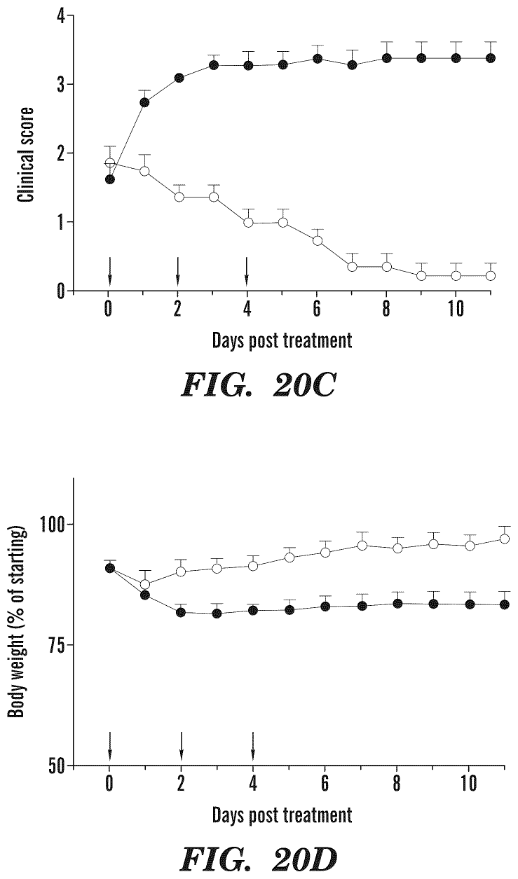

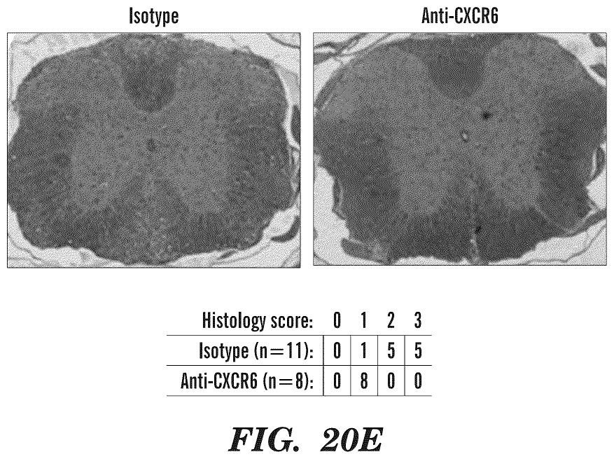

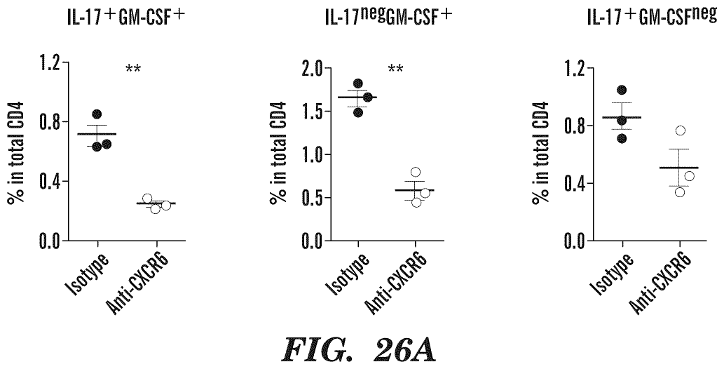

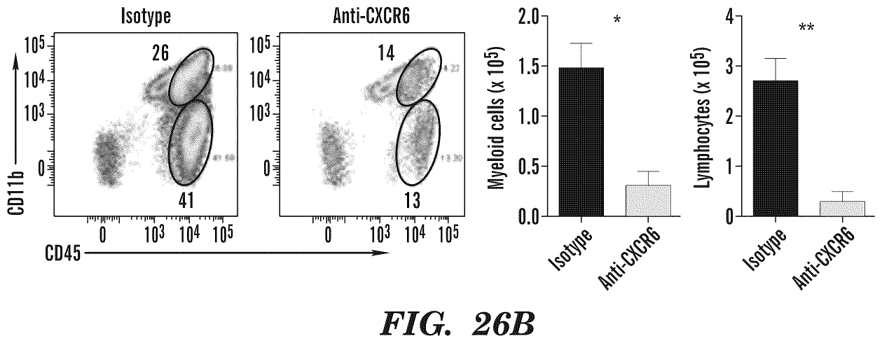

To test the role of CXCR6-marked CD4 cells in EAE, a cell depletion strategy was used. A previous study found that disease is not different in MOG-immunized Cxcr6−/− and wt mice, indicating that the CXCR6 molecule itself is not required for disease (44). To not be bound by a particular theory, it was hypothesized that a CXCR6-directed therapy might be used to deplete encephalitogenic TH cells. In feasibility studies, MOG-immunized wt mice were given a single dose of anti-CXCR6 mAb at disease onset and lymph node cells were examined 24 h later. The decreased frequency of GM-CSF/IFNγ DP and GM-CSF SP CD4 cells compared with isotype-treated mice (FIG. 26A) suggested successful targeting of CXCR6+CD4 cells. Treating immunized mice with 3 doses of anti-CXCR6 mAb starting before appearance of symptoms (‘prevention protocol’) largely abrogated clinical disease (FIGS. 20A, 20B), and fewer lymphocytes and myeloid cells infiltrated the spinal cord (FIG. 26B). Moreover, delivering anti-CXCR6 mAb after appearance of symptoms (‘therapeutic protocol’) reversed the clinical score to baseline (FIG. 20C), prevented body weight loss (FIG. 20D), and dramatically diminished the histology score and leukocyte accumulation in the spinal cord (FIG. 20E).

### CXCR6 Identifies Pathogenic TH Cells in Different Autoimmune Disorders

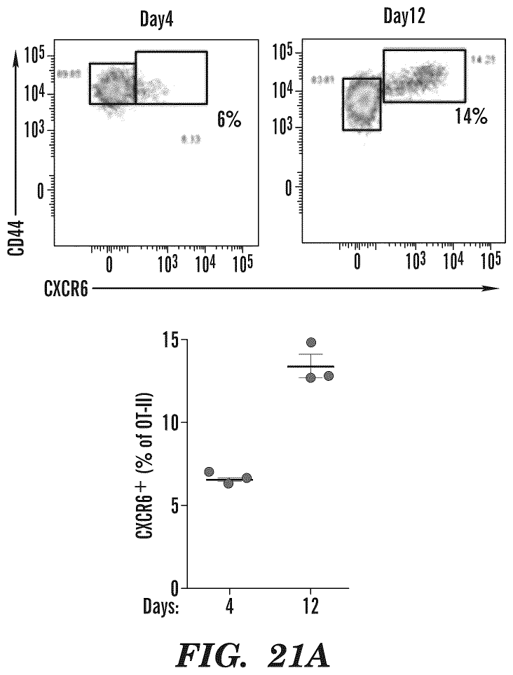

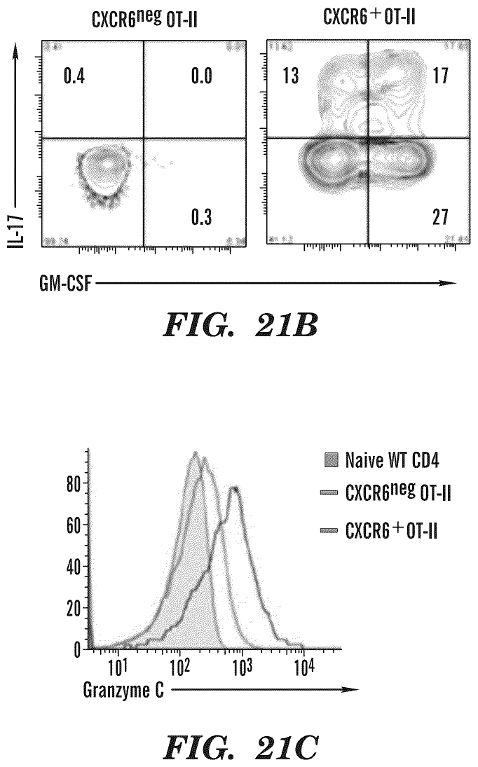

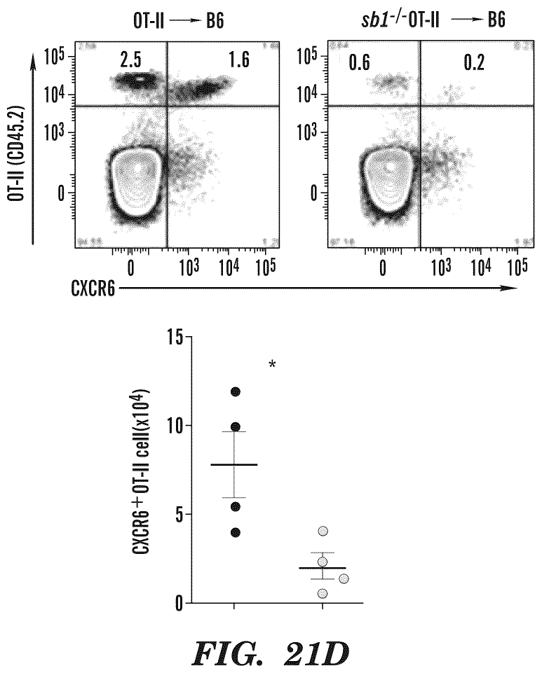

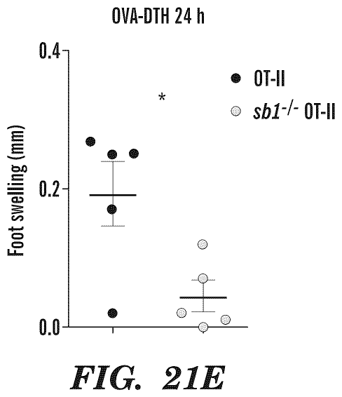

CXCR6 also marks an expanded population of CD4 cells expressing multiple cytokines and GzmC in mice adoptively transferred with OT-II cells and immunized with ovalbumin peptide (OVA) (FIGS. 21A-21C). In the absence of Sb1, the expanded population of CXCR6+OT-II cells was largely abrogated (FIG. 21D), and pathogenic function was lacking as indicated by decreased footpad swelling on OVA challenge in the footpad (DTH response) (FIG. 21E). A blunted DTH response was seen also in MOG-immunized Sb1−/− mice challenged in the footpad with MOG peptide (FIG. 17F).

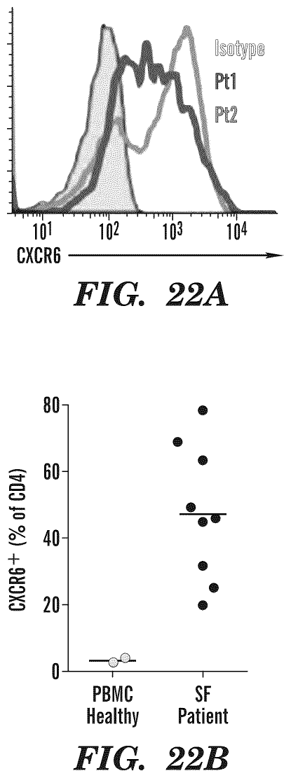

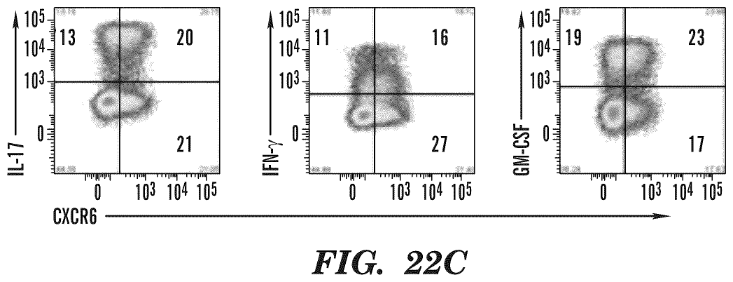

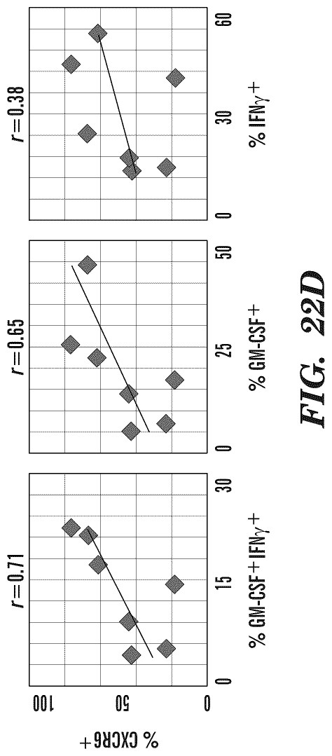

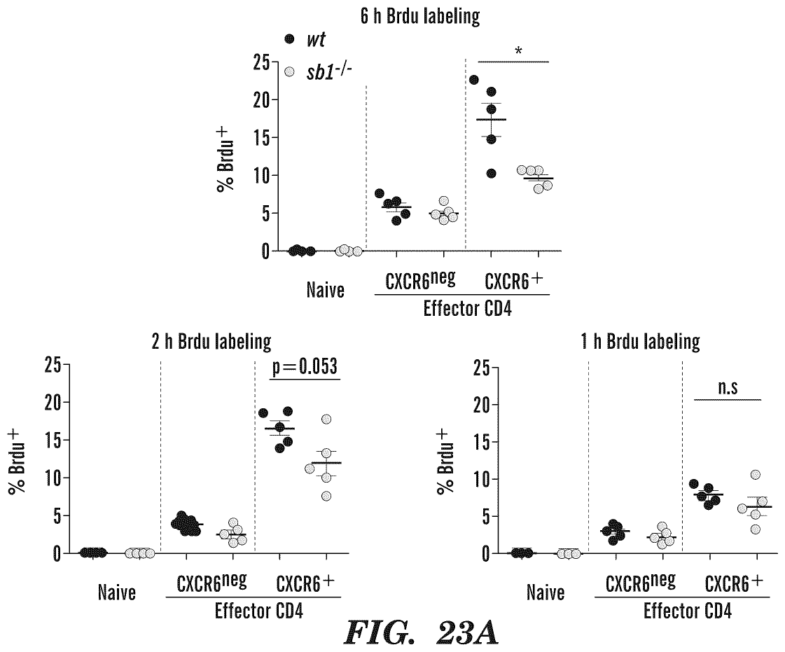

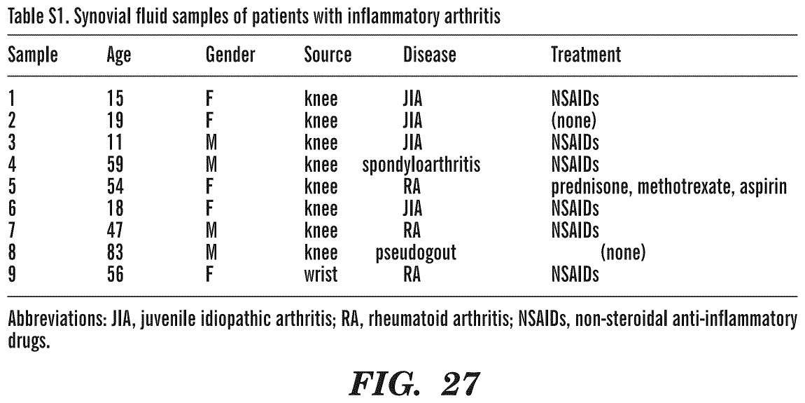

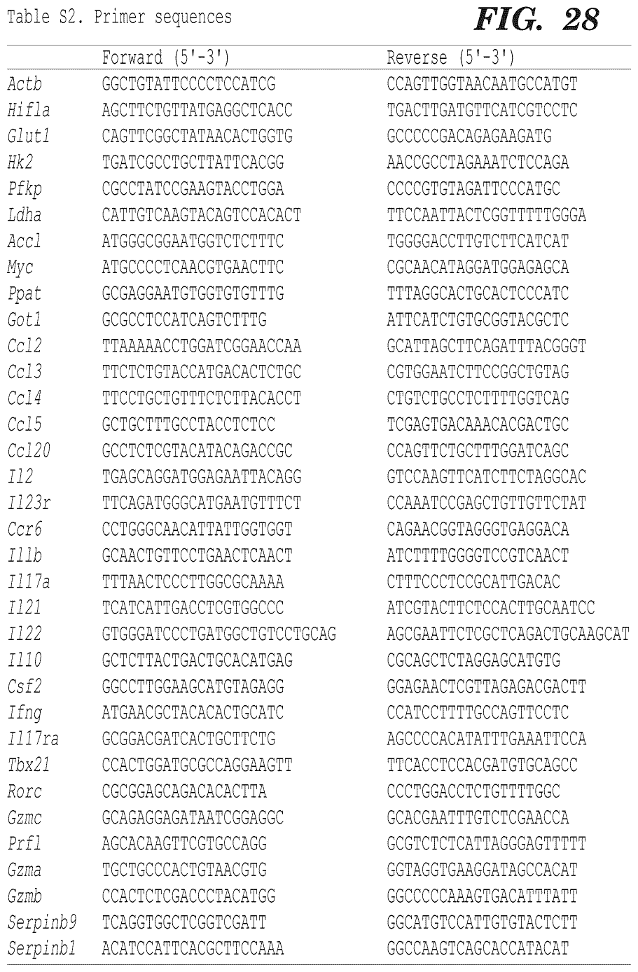

T cells of synovial fluid (SF) of inflammatory arthritis patients were also evaluated (Table 1, FIG. 27) and found that CXCR6+CD4 cells were highly enriched (FIG. 22A, 22B). The proportions of CXCR6+CD4 cells correlated well with the proportions of GM-CSF/IFNγ DP and GM-CSF SP cells, and not with IFNγ SP cells (FIGS. 22C, 22D). Thus, in both mouse and human TH17-driven autoimmune disorders, CXCR6 identifies CD4 cells that produce multiple key pathogenic cytokines and are enriched in inflamed tissues.

TABLE 1
Synovial fluid samples of patients with inflammatory arthritis

| Sample | Age | Gender | Source | Disease | Treatment |
|---|---|---|---|---|---|
| 1 | 15 | F | knee | JIA | NSAIDs |
| 2 | 19 | F | knee | JIA | (none) |
| 3 | 11 | M | knee | JIA | NSAIDs |
| 4 | 59 | M | knee | spondyloarthritis | NSAIDs |
| 5 | 54 | F | knee | RA | prednisone, |
| | | | | | methotrexate, |
| | | | | | aspirin |
| 6 | 18 | F | knee | JIA | NSAIDs |
| 7 | 47 | M | knee | RA | NSAIDs |
| 8 | 83 | M | knee | pseudogout | (none) |
| 9 | 56 | F | wrist | RA | NSAIDs |

Abbreviations: JIA, juvenile idiopathic arthritis; RA, rheumatoid arthritis; NSAIDs, non-steroidal anti-inflammatory drugs.

### Sb1 Controls the Longevity of CXCR6+ Encephalitogenic TH Cells

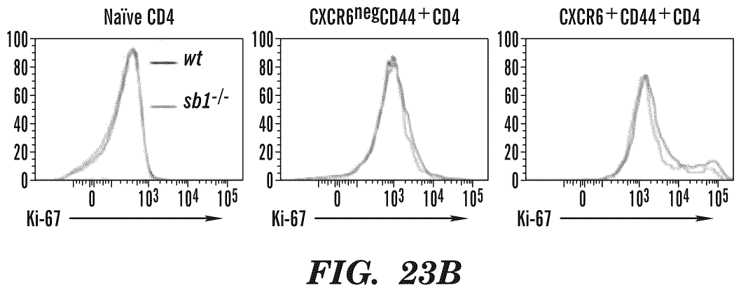

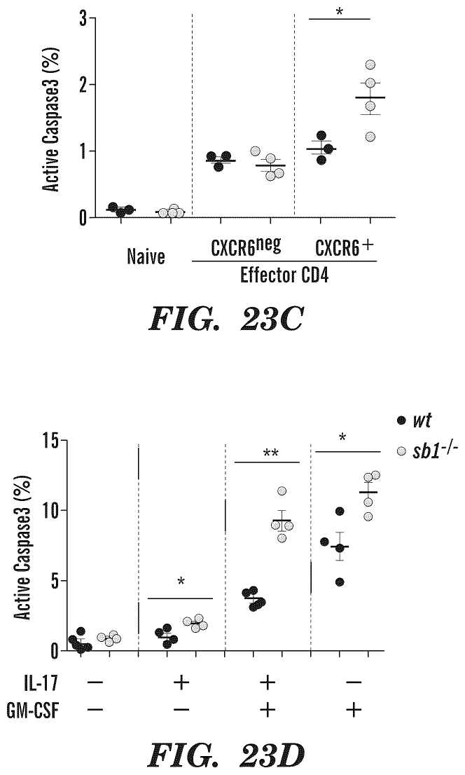

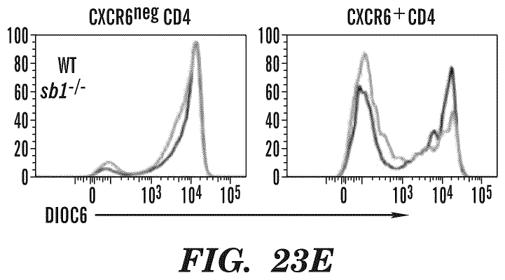

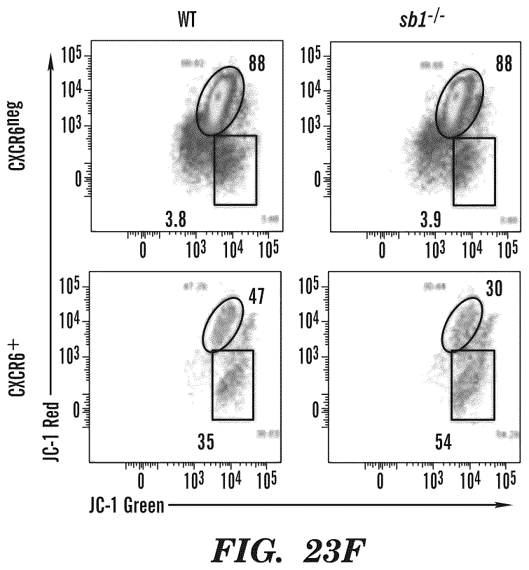

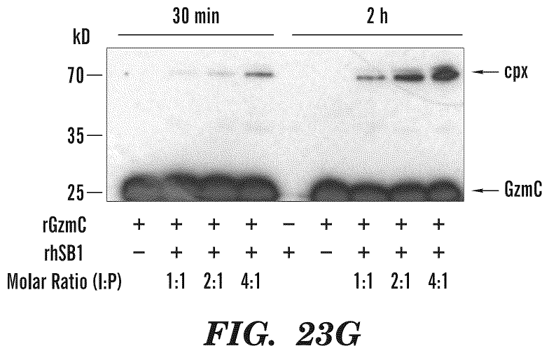

Having established molecular and functional features of the serpinB1-dependent CXCR6+CD4 cells, the mechanism to account for their deficiency in Sb1−/− mice was investigated. MOG-immunized wt and Sb1−/− mice were injected at disease onset with the thymidine analog bromodeoxyuridine (BrdU) to label proliferating cells. Analysis of lymph node cells 6 hr later revealed that (i) CXCR6+CD4 cells have higher BrdU labeling than CXCR6neg cells, suggesting that CXCR6+ cells have a high proliferation rate and (ii) the frequency of BrdU+Sb1−/− CXCR6+ cells was decreased compared with that of BrdU+ wt CXCR6+ cells. Because the frequency of cells labeled with BrdU after a fixed time span can be affected by both cell death as well as cell proliferation, the labeling time was shortened to minimize effects of cell death. After 2 h, the frequency of BrdU+ wt cells was unchanged, but the deficit of BrdU+ Sb1−/− CXCR6+CD4 cells was largely diminished, and at 1 h the frequency of BrdU+ Sb1−/− CXCR6+CD4 cells was not different from corresponding wt cells, indicating that Sb1−/− and wt CXCR6+CD4 cells proliferate at the same rate (FIG. 23A). Further studies comparing the two genotypes for staining with Ki-67, a nuclear marker of recently proliferated cells, provided verifying evidence that proliferation of CXCR6+CD4 cells is rapid and is not different between Sb1−/− and wt mice (FIG. 23B).

To examine cell death, freshly isolated LN cells were stained for active caspase-3. Caspase-3+ cells, although few in number, were significantly increased among Sb1−/− CXCR6 CXCR6+CD4 cells compared to wt CXCR6+CD4 cells (FIG. 23C). Because dead cells bearing active caspase 3 are rapidly removed in vivo, the measurement was repeated after stimulation of the cells ex vivo, conditions less favorable to dead cell removal. After ex vivo stimulation, the excess of active caspase-3+ Sb1−/− cells over wt cells was substantial, especially for IL-17/GM-CSF DP and GM-CSF SP cells (FIG. 23D).

It was contemplated whether the serpinB1-dependent CD4 cells are subject to self-inflicted cell death as occurs in other granule-containing cells such as NK cells, CD8 cells and neutrophils (22, 34, 35, 45). In this mechanism, high level activation or stress causes permeabilization of granule membranes allowing granzymes to leak into the cytoplasm (46). GzmB, a serine protease released in cytolytic CD8 cells and NK cells, can induce cell suicide, but this is opposed by the cytoprotective inhibitor. In neutrophils, cell death is mediated by the azurophil granule proteases cathepsin G and proteinase-3 (PR3) and is opposed by SerpinB1, which irreversibly inactivates both of these serine proteases (34, 35).

Because loss of mitochondrial membrane potential (Δψm) is an early and irreversible step of this intrinsic death process (47), the mitochondrial dye DiOC6 was used to measure Δψm at disease onset. More than 80% of CXCR6neg CD4 cells of wt and Sb1−/− mice had high retention of DiOC6, indicating intact mitochondria. In contrast, a substantial percentage of wt CXCR6+CD4 cells and an even greater percentage of Sb1−/− CXCR6+CD4 cells had low dye retention, indicating mitochondrial damage and irreversible commitment to cell death (FIG. 23E). These findings were confirmed in studies with the independent mitochondrial probe JC-1 (FIG. 23F). Overall, the findings indicate that SerpinB1, by preventing cell suicide, determines whether sufficient CXCR6+CD4 cells survive to form an expanded population capable of implementing pathogenesis.

Further study will be required to fully document the death process and identify the SerpinB1-inhibitable protease (or proteases) responsible for the death of CXCR6+CD4 cells, but the expression data suggest GzmC, a serine protease that has a cytolytic efficiency comparable to GzmB and acts via a cell death pathway involving direct mitochondrial damage (48). Thus, GzmC, a chymotryptase, is directly inhibited by SerpinB1 as indicated by the covalent complex formed on incubating GzmC with human SerpinB1 (FIG. 23G). Neither GzmA, a tryptase, nor GzmB, an aspase, can be inhibited by SerpinB1(27).

### SUMMARY

Provided herein is the discovery that the protease inhibitor SerpinB1 is expressed at the onset of EAE in a subset of peripheral effector CD4 cells that was subsequently identified as the paralysis-inducing T cells. Furthermore, it was also discovered that serpinB1 is required for survival and expansion but not generation of these cells. On deletion of serpinB1, encephalitogenic TH cells do not accumulate in the CNS of immunized mice, and disease is substantially ameliorated. Findings from transcriptomics attesting to the unusual nature of these TH cells include the newly identified signature genes GzmC, GzmA and PrfA and the previously documented Csf2 and Ifng. These TH cells are distinguished also by the presence of cytolytic granules along with the previously documented secretion system for multiple cytokines. Important also is the finding that CXCR6, a chemokine receptor, is suitable as a cell surface marker of the serpinb1-dependent encephalitogenic TH cells. Having a marker that identifies the truly encephalitogenic T cells in EAE paves the way for design of novel therapy for human MS.

It is generally accepted that the function of CD4 cells in MS and related autoimmune disorders is not fully explained by the action of polarized TH1 or TH17 cells, but rather by cells generated through a not-yet-characterized encephalitogenic program initiated and maintained by IL10 and IL23 (reviewed in (49)). A link of serpinB1 with the encephalitogenic program was strongly suggested by finding indistinguishable phenotypes for Sb1-deficient and 1123r-deficient mice (FIGS. 16 and 17 and ref (11)). The cumulative findings for these mice suggest that serpinB1 functions downstream of IL-23 to regulate the encephalitogenic program, which has at its core function, the successful expansion of a select subset of primed TH cells. In the program, serpinB1 restricts a proliferation-associated granule protease-mediated mitochondrial damage/suicidal death pathway and thus is crucial for survival and expansion of the select T helper cells that constitute the encephalitogenic population. Altogether, the findings describe encephalitogenic TH cells as cells that produce multiple pathogenic cytokines especially GM-CSF, proliferate rapidly, rely on serpinB1 to survive during rapid expansion, express cytotoxic granule components perforin A, GzmA and GzmC and are marked by CXCR6.

TH cells expressing most of the features of encephalitogenic TH cells, specifically CXCR6+, multiple cytokines, granzymes, pathogenic function, IL23-dependence, were found in the OT-II transfer model of DTH, indicating that the disease-inducing TH cells described here are not limited to autoimmune neuroinflammation.

TH cells with similarities to murine serpinB1-dependent encephalitogenic TH cells were found also in other autoimmune disorders. The first were the IL-17/IFNγ DP CD4 cells noted in the gut of Crohn's disease patients (40) and later in brain tissue of MS patients (50). In MS, myelin-reactive cytokine-producing CD4 cell clones were characterized as IL-17/GM-CSF DP, GM-CSF SP and IFNγ SP (41), a pattern similar to murine encephalitogenic TH cells. It is now appreciated that IL-17/IFNγ DP CD4 cells, known as TH1/TH17 and TH17/TH1 cells, and also a subset of IFNγ SP CD4 cells, are not TH1 cells but rather are derived from TH17 cells (15, 51).

An earlier study found synovial fluids (SF) of inflammatory arthritis patients enriched in CXCR6+ CD4 cells that produce IFNγ and, on that basis, were reported as TH1 cells (52). This led to the notion that CXCR6 marks inflammatory TH1 cells at tissue sites. The data showed in this study that the CD4 cells marked by CXCR6 in inflammatory arthritis SF are enriched in cytokine DP (GM-CSF/IL-17 and GM-CSF/IFNγ) cells, suggesting their relatedness to the TH17-derived encephalitogenic TH cells in murine EAE (FIG. 22). Relatedness is suggested also for IL17/IFNγ DP cells that emerge in an IL-23 dependent fashion in murine inflammatory bowel disease (53). In a T cell transfer model of chronic colitis, pathogenic CD4 cells marked by CXCR6 include IL-17/IFNγ DP cells along with predominant IL-17 SP and IFNγ SP cells (54); poor proliferation distinguishes these cells from the rapidly proliferating CXCR6+ TH cells in EAE.

Lastly, encephalitogenic CXCR6+ TH cells have at least one feature, cytotoxic granules, in common with CD4+ cytolytic T cells (CD4+ CTL) that provide e.g., antiviral protection. Recent work showed that progression of disease in MS patients correlates with the density of circulating CD4+ CTL (55). Further work will be needed to determine the relatedness of these multiple cytokine-producing and granzyme-expressing CD4 cells in autoimmune and chronic inflammatory diseases.

To determine how serpinB1 regulates the density of encephalitogenic TH cells in EAE mice, cell proliferation and cell death were evaluated. Multiple approaches to proliferation including in vivo BrdU labeling showed that the proliferation rate for encephalitogenic TH cells is not different in Sb1−/− and wt mice. Cell death quantitation was challenging because dead cells are rapidly removed in vivo, and thus there are few cells to count. The most definitive experiments involved quantifying cells in the process of dying, i.e., cells irreversibly committed to death due to mitochondrial damage (47), a process induced by leakage of cytotoxic granule contents (22). This approach demonstrated (i) robust ongoing death of wt encephalitogenic TH cells occurring concurrent with robust proliferation and (ii) further increase of dying encephalitogenic TH cells in mice lacking serpinB1. The cumulative findings indicate that the extent of expansion of CXCR6+ TH cell subset in EAE and hence their encephalitogenicity is the net result of simultaneous robust proliferation and robust cell death, the latter restricted by serpinB1 and increased in its absence. The factors driving evolution of this inherently inefficient cell expansion mechanism are unknown, but it is contemplated that they reflect the biological need for highly potent cell populations to be tightly and irreversibly regulated.

Of note, the serpinB1-mediated mechanism proposed here as the basis of the IL-1β and IL-23 driven TH cell encephalitogenic program, although new for CD4 cells, is not unique, but rather is analogous to the programs that control expansion and retraction of human and mouse populations of activated CD8 cytolytic cells and NK cells (22, 45). In a similar program, SerpinB1 stoichiometrically interacts with endogenous granule proteases to control survival of human and mouse neutrophils (34, 35).

Finally, apparent depletion of CXCR6+ cells by anti-CXCR6 treatment of immunized mice prevented the development of clinical disease and decreased the accumulation of cytokine-producing TH cells in the spinal cord and reversed or ameliorated clinical symptoms in diseased mice. These findings indicate that the serpinB1-dependent multifunctional cells described here indeed mediate encephalitogenicity in EAE. They suggest that therapies to regulate serpinB1 levels or, more realistically, strategies to deplete CXCR6-marked TH cells can mitigate autoimmune disorders such as MS.

### Example 4: Materials and Methods

### Study Design

This is combined experimental laboratory study performed with living mice and mouse cells and a non-interventional study of human inflammatory T cells of patients. The objective was to uncover the mechanism whereby serpinB1, expressed in a subset of CD4 T cells, drives autoimmunity and to determine whether that mechanism, once understood, could be exploited for therapeutic purposes to ameliorate autoimmune disorders like MS and inflammatory arthritis. Study components were not predefined. The number of mice and number of replicates for each study is indicated in the figure legends. Mechanistic studies on mouse cells were generally performed without blinding. The pathology scoring of spinal cord specimens was done by a pathologist who was presented with coded samples in random order. In each experiment, the various groups of mice were the same gender and matched for age and body weight. All major studies were performed in both males and females, and no gender-specific differences were detected. For time course experiments, mice were assigned to groups prior to start of experiment. Special precautions for randomization in the therapeutic treatment of MOG-immunized mice are detailed in the Fig legends. Synovial fluid of patients with inflammatory arthritis bore coded identifiers at the time of study. Information on diagnosis and other disease parameters (Table 2, FIG. 28) became available only after completion of assays and analysis of data.

### Human Samples

Discarded synovial fluid specimens were obtained from patients with inflammatory arthritis undergoing diagnostic and/or therapeutic arthrocentesis for active joint inflammation. Associated clinical information was obtained from medical record review within 2 wk of sample collection, before associated identifying linkers were destroyed. Information on the patients is provided in Table 1. In brief, synovial fluid specimens were diluted in RPMI-1640 medium containing 10% FCS and then centrifuged at 300 g for 10 min. Single cell suspensions were prepared for surface staining or were stimulated with PMA and ionomycin (P+I) in the presence of Brefeldin A for 4 h to detect cytokines.

### Mice

SerpinB1 deficient mice (serpinb1a−/−, hereafter Sb1−/−) were generated in 129S6/SvEv/Tac (129S6) background (33) and were backcrossed to C57BL/6J (B6) (CD45.2+) background for more than 10 generations. Congenic B6.SJL-CD45.1 (CD45.1, wt), OT-II, CD4-Cre, and Rag1/−mice were from the Jackson Laboratory. CD45.1 sb1−/− and OT-II sb1−/− strains were generated by mating Sb1−/− B6 mice with CD45.1 or OT-II mice and intercrossing the resulting heterozygotes. Il23rfl/fl mice were originally described in Aden et al (58). Il23rΔCD4 mice were generated by mating Il23rfl/fl mice (58) with Cd4cre mice and intercrossing the resulting heterozygotes. Mice were maintained in the animal facility of Boston Children's Hospital or the animal facility of Institute of Experimental Immunology, University of Zurich. Animal studies were approved by the Institutional Animal Care and Use Committee of Boston Children's Hospital or the cantonal veterinary office of Zurich. To generate mixed wt:Sb1−/− bone marrow chimeras, Rag1/− mice were lethally irradiated with two doses of 550 rads separated by a 4 h interval. T cell-depleted wild type and mutant bone marrow cells with traceable congenic CD45 markers were mixed at 1:1 ratio, and injected i.v. To generate mixed wt:Il23rΔCD4 bone marrow chimeras, a total of 5×10⁶ bone marrow cells from wt (CD45.1) and Il23rΔCD4 (CD45.2) mice were injected in the tail veil of wt CD45.1×CD45.2 mice irradiated 2×550 rad with a 24 h interval. To prevent bacterial infection, the mice were provided with autoclaved drinking water containing Sulfatrim 1 wk before until 4 wk after irradiation or 0.2% (vol/vol) Borgal was added to the drinking water for 2 wk.

### T Helper Cell Differentiation

Single cell suspensions were prepared from spleens of 4-6 wk old B6 mice. Naïve CD4 T cells (CD4+CD25negCD44negCD62L+) were FAC-sorted, and in vitro polarization of Th0, Th1, Treg and Th17 subsets was conducted as described24. To generate Th2 cells, naïve CD4 T cells were cultured in 24-well plates (Costar) pre-coated with anti-CD3 and anti-CD28 in the presence of mIL-4 (10 ng ml-1, Biolegend) and anti-mIFN-γ (XMG1.2, 5 jig ml-1, BioXcell). For two-stage differentiation(17), freshly differentiated Th17 cells were rested for 2 days in the presence of mIL-2 (2 ng ml-1 and then were collected, washed and re-stimulated with anti-CD3 and anti-CD28 (both 1 jig ml-1, plate coated) in the absence or presence of mIL-2 (20 ng ml-1), mIL-12 (20 ng ml-1) or mIL-23 (50 ng ml-1) for additional 24 h.

### Induction of EAE

Mice were injected with myelin oligodendrocyte glycoprotein (MOG) amino acid 35-55 (ProSpec, 150 jig per mouse) emulsified with complete Freund's adjuvant containing heat-killed Mycobacterium tuberculosis strain H37Ra (4 mg ml-1) (Difco) at three sites on the back and were injected i.p with 200 ng pertussis toxin (List Biological Labs) on days 0 and 2 (hereafter called ‘MOG immunization’). Both male and female mice were used, and in each experiment, the animals being compared were matched for age and gender. Disease was scored as (0) asymptomatic, (1) limp tail, (2) hindlimb weakness, (3) hindlimb paralysis, (4) hindlimb paralysis and partial or complete forelimb paralysis. Mice were euthanized when they reached stage 4 or stage 3 accompanied with 25% bodyweight loss per institutional regulations.

### Adoptive Transfer EAE

MOG-immunized wt or sb1−/− mice were sacrificed late during the “induction phase” prior to development of clinical symptoms (i.e., days 7-10). Lymph nodes and spleen were harvested and cultured with MOG peptide plus IL-23. The expanded CD4 T cells were enriched by negative magnetic chromatography (Miltenyi Biotec) and injected i.v (5×106 cells per mouse) through tail vein into naïve wt or sb1−/− mice. Mice were injected i.p with 200 ng pertussis toxin on days 0 and 2.

### Naïve CD4 Cell Transfer Model of EAE

Naïve CD4 T cells were isolated from spleens of naïve wt or sb1−/− mice by negative magnetic selection (Miltenyi Biotec) and 5×106 cells per mouse were injected in the tail vein of naive Rag1/− recipient mice. One day later, the mice were immunized with MOG35-55/CFA followed by pertussis toxin injection as above to induce EAE.

### OT-II Tracking Study

Congenic WT CD45.1 mice were i.v. transferred with 2×105 naïve CD45.2+OT-II cells or naïve CD45.2+sb1−/−OT-II cells and s.c. immunized with OVA323-339/CFA. Draining lymph nodes were harvested on days 4 and 12 post immunization, and OT-II cells were quantified and phenotyped by flow cytometry.

### DTH Reaction

Wt recipients of OT-II cells or sb1−/−OT-II cells were immunized with OVA323-339/CFA and were challenged in one hind footpad with 50 jig OVA323-339 in saline as described(11). For the MOG-DTH reaction, wt or sb1−/− mice were immunized with MOG according to the EAE-induction protocol and, in the pre-disease phase, were challenged in one hind footpad with 50 jig MOG35-55 in saline. In both cases, the contralateral footpad was injected with saline. Foot thickness was measured with calipers; swelling is calculated by subtracting the foot thickness prior to challenge.

### Isolation of Spinal Cord-Infiltrating Cells

EAE mice were sacrificed, and spinal cords were removed. Tissues were mechanically dissociated and digested for 30 min at 37° C. by 1 mg ml-1 collagenase D (Sigma-Aldrich) and 50 unit/ml DNAse I (Roche) in complete RPMI 1640 medium containing 5% FCS. Leukocytes were further enriched by 30% versus 80% percoll gradient.

### Spinal Cord Histology

Spinal cords were fixed by immersion in Bouin's solution (Sigma-Aldrich) and were embedded in paraffin wax. Sections were cut from various locations and stained with H & E. Sections were evaluated by a pathologist and scored for severity of inflammation and degeneration as (0) asymptomatic, (1) mild, (2) moderately severe, (3) severe. Scoring was done blindly.

### Intracellular Staining and Flow Cytometry

Cells were stimulated for 4 h with PMA (50 ng ml-1) and ionomycin (750 ng/ml) (Sigma-Aldrich) in the presence of Brefeldin A (P+I stimulation). Cells were stained with fluorochrome-conjugated antibodies to surface markers. After washing, cells were fixed for flow cytometry analysis, or were permeabilized and stained intracellularly with fluorochrome-conjugated antibodies using fixation/permeabilization reagents and protocols from BD Bioscience. In case of LAMP1 staining, anti-LAMP1 antibody was added into the culture at the beginning of P+I stimulation. Fluorochrome-conjugated antibodies or cell death related dyes are: from Biolegend: FITC- or PE-Cy7-anti-mCD3 (145-2C11), Pacific blue- or APC-anti-mCD45.1 (A20), Pacific blue- or PE-anti-mCD45 (30-F11), Pacific blue-anti-mCD45.2 (104), Pacific blue- or PE-Cy7- or APC-anti-mCD4 (GK1.5), Alexa fluor488-anti-Brdu (3D4), PE-anti-mGranzymeC (SFC1D8), FITC-anti-h/mGranzymeB (GB1), PE-anti-mIL1R1 (JAMA-147), FITC-anti-mCD44 (IM7), APC-anti-mCXCR6 (SA051D1), APC-anti-mCCR2 (SA203G11), APC- or PE-Cy7-anti-mCCR6 (29-2L17), PE-Cy7-anti-mCD11b (M1/70), Alexa Fluor488-anti-mGr1 (RB6-8C5), FITC-anti-mIntergrinβ2 (M18/2), PE-anti-mIntegrinαL (M17/4), PE-anti-mCD25 (3C7), PE-anti-mIL7Rα (A7R34), FITC-anti-h/m/rat ICOS (C398.4A), PE-Cy7-anti-mPD1 (29F.1A12), PE-anti-mCD62L (MEL-14), Pacific blue- or APC-anti-mIL-17 (TCI 1-18H10.1), Pacific blue- or PE-anti-mIFN-γ (XMG1.2), FITC- or PE-anti-mGM-CSF (MP1-22E9), Alexa Fluor488-anti-FoxP3 (FJK-16s). From eBioscience: APC-anti-perforin (eBioOMAK-D), PE-anti-LAMP1 (1D4B), PE-Cy7-anti-Ki67 (S01A15), FITC-anti-mIntegrin β1 (eBioHMb1-1), PE-anti-mIntegrinα4 (R1-2), FITC-anti-mIntegrinβ3 (2C9.G3), PE-anti-mIntegrinαV (RMV-7). From R&D system: PE-anti-mIL-23R (753317). From BD Biosciences: PE-anti-mCD69 (H1.2F3), FITC-rabbit-anti-active caspase3 (C92-605), FITC-Annexin V. Data were acquired on a Canto II cytometer (BD Biosciences) and analyzed using FlowJo software (Tree Star).

### BrdU Labeling and Detection

Mice were immunized to induce EAE. At day 10 post EAE induction, BrdU (1 mg/mouse) was i.v. injected or i.p. injected. Lymph node cells were harvested and stained for surface expression of various markers, and detection of BrdU was carried out following the manufacturer's protocol (BD PharMingen). To monitor the BrdU incorporation in cytokine producing cells, mice were i.p injected with Brdu (1 mg/mouse) for 6 h. Then, lymph node cells were stimulated for 2.5 hr with PMA (50 ng ml-1) and ionomycin (750 ng ml-1) (Sigma-Aldrich) in the presence of Brefeldin A, followed by surface marker staining, and intracellular co-staining of cytokine and BrdU.

### Mitochondrial Membrane Potential

Freshly harvested lymph node leukocytes were incubated with 3,3′-dihexyloxacarbocyanine iodide (DIOC6)(47) (10 nM, Sigma-Aldrich) for 15 min at 37° C., washed, and stained with fluorescein-labeled mAbs. The cells were evaluated by flow cytometry without fixation. Alternatively, 5,5′,6,6′-tetrachloro-1,1′,3,3′-tetraethylbenzimidazolocarbo-cyanine iodide (JC-1)(59) (2 jig ml-1) (Thermo Fisher) was used as mitochondrial probe in the same protocol.

### Western Blot

Samples were resolved on 12% Tris-glycine gels and transferred onto PVDF. Membranes were blocked with 5% or 20% milk solids and stained with rabbit antiserum generated to human SerpinB1 or IgG fraction (arC70688) of rabbit 428A antiserum to granzyme-C36 followed by HRP-conjugated secondary antibodies (Cell Signaling or BioRad). Bands were visualized by enhanced chemiluminescence (ECL Plus, Amersham Biosciences or West Pico, Pierce). SerpinB1 blots were stripped and restained with rabbit mAb to GAPDH (Cell Signaling).

### Serpin-Protease Complex Formation

Recombinant E193G-granzymeC (60) (20 ng ml-1) was co-incubated with 1, 2 or 4 molar equivalents of recombinant human SerpinB137. The reactions were prepared for Western blot (as above) by heating with SDS and 2-mercaptoethanol. PVDF transfers of parallel reactions were stained for protein using Aurodye (colloidal gold, Amersham).

Reverse Transcription and qPCR Analysis

RNA was isolated using RNeasy Plus kits (74134, Qiagen) according to the manufacturer's protocol and reverse-transcribed using the iScript™ cDNA Synthesis kit (Bio-Rad). The qPCR assays were performed on the CFX96™ Real-Time System (Bio-Rad) with the iTaq™ Universal SYBR Green Supermix (Bio-Rad) using 30 sec denaturation at 95° C. and 40 cycles of 5 sec at 95° C. and 30 sec at 61° C. using the primers (Table 2). Relative expression level for each gene was calculated by using the ΔΔCt method and normalizing to Actb.

### RNA Sequencing

Sb1 related RNAseq: CD4 effector cells (CD44+CD4) were sort-purified from pooled lymph node cells of MOG-immunized wt and sb1−/− mice at disease onset. The cells were stimulated for 4 hr±SEM with P+I, and RNA was purified using QIAGEN RNeasy Plus Mini kits and quantified by optical density at 260/280/230 nm RNA (1 jig per genotype) was shipped to Macrogen Corp (Seoul, Korea) and TruSeq RNA V2 kits were used to construct transcript-specific libraries that were sequenced on Illumina HiSeq2500. The resulting 4.5 Gb/genotype of raw data was trimmed, and 20 million reads were mapped. Of the ≥24,000 genes evaluated in the resulting 2-way data sets, the 9,600 genes that had expression levels (FPKM)≥1.0 were analyzed for differential expression.

IL-23r related RNAseq: Chimeric mice (wt:Il23rΔCD4) mice were immunized with MOG. Nine days later, effector CD4 cells (CD44hiCD62Llo CD4+ T cells) were sorted from lymph nodes (axillary, brachial and inguinal) using the following antibodies: CD45.1 (clone A20), CD11b (M1/70), CD8a (53-6.7) from BD Pharmigen; CD45.2 (104), CD4 (RM4-5), CD62L (MEL-14) from BioLegend; CD3 (17A2) and CD44 (IM7) from eBioscience. Doublets exclusion was performed by FSC-A/FSC-H gating, and cell death exclusion with Zombie Aqua Fixable Viability Kit (BioLegend). Cell sorting was performed on the FACS Aria III (BD Biosciences). Total RNA was isolated with QIAGEN RNeasy Plus Micro Kit according to manufacturer's instructions. For RNA preamplification and library preparation, the Smart-seq2 protocol was used in combination with Illumina's Nextera XT DNA Library Preparation Kit (Illumina). Library preparation and NGS were performed by the Genomics Facility Basel (ETH Zurich and University of Basel, Switzerland) using the HiSeq 2500 v4 System (Illumina). Quality control included the fastqc analysis.

### Anti-CXCR6 Antibody Treatment

Isotype rat IgG2b antibody (RTK4530) and rat-anti-mouse CXCR6 (SA051D1) were from Biolegend. The antibodies (ULEAF purity) were sterile-filtered (0.2 μm filter), contained no preservative, no azide, and endotoxin <0.01 EU/μg protein. Isotype or anti-CXCR6 antibodies were i.p. injected.

### Statistical Analysis

Statistical analyses were performed using Prism 4 (Graphpad Software). Student's t-test, unpaired and paired, and one-way ANOVA were used according to the type of experiment. p-values ≤0.05 were considered significant.

### Example 5: Explanation of Video Footage and Experimental Design

An explanation of videos taken of treated mice are explained herein and include videos 1.mp4, 2.mp4, 3.mp4, 4.mp4 and 5.mp4.

Treatment with anti-CXCR6 reverses established EAE—Behavior of anti-CXCR6-treated and isotype-treated mice. Therapeutic protocol. Nineteen wt mice, 5 cages of 4-5 littermates/cage, were immunized with MOG, and when disease was first detected (clinical score 1-3), the mice were randomly assigned to receive 400 μg i.p. of either anti-CXCR6 antibody or isotype control (n=11) on that day (‘Day 0’) and 2 and 4 days later. Clinical scores were recorded daily beginning on Day 0 (FIG. 20 C). Videos were prepared during a single scoring session corresponding to treatment days 3 to 6, at which time three of the isotype-treated mice had reached score 4 and been sacrificed per protocol. All mice had been identified at weaning by an ear punch system, which was supplemented for videotaping by marker pen labeling of the tail. Marker pen labeling system: #1:one level line; #2: two level lines; #3: Three level lines; #4: Four level lines; #5: one vertical line. Mice in each cage were littermates and remained together throughout the study. In videos of each cage the mice that moved continuously or frequently were mAb treated mice and those that remained in place, in most cases lying prone, or that moved only slowly and infrequently were isotype-treated mice. Detailed information of Isotype-treated and Anti-CXCR6 mAb treated mice were provided in the following.

Video 1 (cage 1197288). Three mice: Anti-CXCR6 mAb, mouse #3, score 2 on Day 0, videotaped on Day 5 (3 treatments) Anti-CXCR6 mAb, mouse #5, score 2 on Day 0, videotaped on Day 4 (2 treatments) Isotype-treated, mouse #1, score 1 on Day 0, videotaped on Day 4 (2 treatments)

Video 2 (cage 1197287). Three mice: Anti-CXCR6 mAb, mouse #4, score 2 on Day 0, videotaped on Day 4 (2 treatments) Isotype-treated, mouse #2, score 3 on Day 0, videotaped on Day 5 (3 treatments) Isotype-treated, mouse #3, score 1 on Day 0, videotaped on Day 3 (2 treatments)

Video 3 (cage 1197271). Three mice: Anti-CXCR6 mAb, mouse #2, score 3 on Day 0, videotaped on Day 6 (3 treatments) Isotype-treated, mouse #1, score 1 on Day 0, videotaped on Day 2 (1 treatment) Isotype-treated, mouse #3, score 2 on Day 0, videotaped on Day 4 (2 treatments)

Video 4 (cage 1197304). Four mice: Anti-CXCR6 mAb, mouse #3, score 2 on Day 0, videotaped on Day 6 (3 treatments) Anti-CXCR6 mAb, mouse #5, score 1 on Day 0, videotaped on Day 4 (2 treatments) Isotype-treated, mouse #1, score 1 on Day 0, videotaped on Day 4 (2 treatments) Isotype-treated, mouse #4, score 1 on Day 0, videotaped on Day 5 (3 treatments)

Video 5 (cage 1197298). Three mice: Anti-CXCR6 mAb, mouse #1, score 2 on Day 0, videotaped on Day 3 (2 treatments) Anti-CXCR6 mAb, mouse #3, score 1 on Day 0, videotaped on Day 6 (3 treatments) Isotype-treated, mouse #2, score 2 on Day 0, videotaped on Day 4 (2 treatments).

The videos 1-5 revealed that mice that moved continuously or frequently were mAb treated mice and those that remained in place, in most cases lying prone, or that moved only slowly and infrequently were isotype-treated mice. These results show the surprisingly result that delivering anti-CXCR6 mAb after appearance of symptoms to the animals (‘therapeutic protocol’) reversed the clinical score to baseline.

### REFERENCES

1. A. Compston, A. Coles, Multiple sclerosis. Lancet 372, 1502-1517 (2008).
2. M. El-behi, A. Rostami, B. Ciric, Current views on the roles of Th1 and Th17 cells in experimental autoimmune encephalomyelitis. J Neuroimmune Pharmacol 5, 189-197 (2010).
3. B. M. Segal, E. M. Shevach, IL-12 unmasks latent autoimmune disease in resistant mice. J Exp Med 184, 771-775 (1996).
4. D. J. Cua, J. Sherlock, Y. Chen, C. A. Murphy, B. Joyce, B. Seymour, L. Lucian, W. To, S. Kwan, T. Churakova, S. Zurawski, M. Wiekowski, S. A. Lira, D. Gorman, R. A. Kastelein, J. D. Sedgwick, Interleukin-23 rather than interleukin-12 is the critical cytokine for autoimmune inflammation of the brain. Nature 421, 744-748 (2003).
5. B. M. Segal, B. K. Dwyer, E. M. Shevach, An interleukin (IL)-10/IL-12 immunoregulatory circuit controls susceptibility to autoimmune disease. J Exp Med 187, 537-546(1998).
6. C. L. Langrish, B. S. McKenzie, N. J. Wilson, R. de Waal Malefyt, R. A.

Kastelein, D. J. Cua, IL-12 and IL-23: master regulators of innate and adaptive immunity. Immunol Rev 202, 96-105 (2004).
7. C. L. Langrish, Y. Chen, W. M. Blumenschein, J. Mattson, B. Basham, J. D. Sedgwick, T. McClanahan, R A. Kastelein, D. J. Cua, IL-23 drives a pathogenic T cell population that induces autoimmune inflammation. J Exp Med 201, 233-240 (2005).
8. S. Haak, A. L. Croxford, K. Kreymborg, F. L. Heppner, S. Pouly, B. Becher, A. Waisman, IL-17A and IL-17F do not contribute vitally to autoimmune neuro-inflammation in mice. J Clin Invest 119, 61-69 (2009).
9. Y. Komiyama, S. Nakae, T. Matsuki, A. Nambu, H. Ishigame, S. Kakuta, K. Sudo, Y. Iwakura, IL-17 plays an important role in the development of experimental autoimmune encephalomyelitis. J Immunol 177, 566-573 (2006).
10. K. Ghoreschi, A. Laurence, X. P. Yang, C. M. Tato, M. J. McGeachy, J. E. Konkel, H. L. Ramos, L. Wei, T. S. Davidson, N. Bouladoux, J. R Grainger, Q. Chen, Y. Kanno, W. T. Watford, H. W. Sun, G. Eberl, E. M. Shevach, Y. Belkaid, D. J. Cua, W. Chen, J. J. O'Shea, Generation of pathogenic T(H)17 cells in the absence of TGF-beta signalling. Nature 467, 967-971 (2010).
11. M. J. McGeachy, Y. Chen, C. M. Tato, A. Laurence, B. Joyce-Shaikh, W. M. Blumenschein, T. K. McClanahan, J. J. O'Shea, D. J. Cua, The interleukin 23 receptor is essential for the terminal differentiation of interleukin 17-producing effector T helper cells in vivo. Nat Immunol 10, 314-324 (2009).
12. C. Sutton, C. Brereton, B. Keogh, K. H. Mills, E. C. Lavelle, A crucial role for interleukin (IL)-1 in the induction of IL-17-producing T cells that mediate autoimmune encephalomyelitis. J Exp Med 203, 1685-1691 (2006).
13. F. Ronchi, C. Basso, S. Preite, A. Reboldi, D. Baumjohann, L. Perlini, A. Lanzavecchia, F. Sallusto, Experimental priming of encephalitogenic Th1/Th17 cells requires pertussis toxin-driven IL-1beta production by myeloid cells. Nat Commun 7, 11541 (2016).
14. Y. Chung, S. H. Chang, G. J. Martinez, X. O. Yang, R. Nurieva, H. S. Kang, L. Ma, S. S. Watowich, A. M. Jetten, Q. Tian, C. Dong, Critical regulation of early Th17 cell differentiation by interleukin-1 signaling. Immunity 30, 576-587 (2009).
15. K. Hirota, J. H. Duarte, M. Veldhoen, E. Homsby, Y. Li, D. J. Cua, H. Ahlfors, C. Wilhelm, M. Tolaini, U. Menzel, A. Garefalaki, A. J. Potocnik, B. Stockinger, Fate mapping of IL-17-producing T cells in inflammatory responses. Nat Immunol 12, 255-263 (2011).
16. L. Codarri, G. Gyulveszi, V. Tosevski, L. Hesske, A. Fontana, L. Magnenat, T. Suter, B. Becher, RORgammat drives production of the cytokine GM-CSF in helper T cells, which is essential for the effector phase of autoimmune neuroinflammation. Nat Immunol 12, 560-567 (2011).
17. M. El-Behi, B. Ciric, H. Dai, Y. Yan, M. Cullimore, F. Safavi, G. X. Zhang, B. N. Dittel, A. Rostami, The encephalitogenicity of T(H)17 cells is dependent on IL-1- and IL-23-induced production of the cytokine GM-CSF. Nat Immunol 12, 568-575 (2011).
18. J. L. McQualter, R Darwiche, C. Ewing, M. Onuki, T. W. Kay, J. A. Hamilton, H. H. Reid, C. C. Bernard, Granulocyte macrophage colony-stimulating factor: a new putative therapeutic target in multiple sclerosis. J Exp Med 194, 873-882 (2001).
19. J. Sun, C. H. Bird, V. Sutton, L. McDonald, P. B. Coughlin, T. A. De Jong, J. A. Trapani, P. I. Bird, A cytosolic granzyme B inhibitor related to the viral apoptotic regulator cytokine response modifier A is present in cytotoxic lymphocytes. J Biol Chem 271, 27802-27809 (1996).
20. T. Phillips, J. T. Opferman, R Shah, N. Liu, C. J. Froelich, P. G. Ashton-Rickardt, A role for the granzyme B inhibitor serine protease inhibitor 6 in CD8+ memory cell homeostasis. J Immunol 173, 3801-3809 (2004).
21. M. Zhang, S. M. Park, Y. Wang, R. Shah, N. Liu, A. E. Murmann, C. R Wang, M. E. Peter, P. G. Ashton-Rickardt, Serine protease inhibitor 6 protects cytotoxic T cells from self-inflicted injury by ensuring the integrity of cytotoxic granules. Immunity 24, 451-461 (2006).
22. C. H. Bird, V. R Sutton, J. Sun, C. E. Hirst, A. Novak, S. Kumar, J. A. Trapani, P. I. Bird, Selective regulation of apoptosis: the cytotoxic lymphocyte serpin proteinase inhibitor 9 protects against granzyme B-mediated apoptosis without perturbing the Fas cell death pathway. Mol Cell Biol 18, 6387-6398 (1998).
23. C. H. Bird, M. E. Christensen, M. S. Mangan, M. D. Prakash, K. A. Sedelies, M. J. Smyth, I. Harper, N. J. Waterhouse, P. I. Bird, The granzyme B-Serpinb9 axis controls the fate of lymphocytes after lysosomal stress. Cell Death Differ 21, 876-887 (2014).
24. M. S. Mangan, C. R. Melo-Silva, J. Luu, C. H. Bird, A. Koskinen, A. Rizzitelli, M. Prakash, K. L. Scarff, A. Mullbacher, M. Regner, P. I. Bird, A pro-survival role for the intracellular granzyme B inhibitor Serpinb9 in natural killer cells during poxvirus infection. Immunol Cell Biol 95, 884-894 (2017).
25. D. Kaiserman, P. I. Bird, Control of granzymes by serpins. Cell Death Differ 17, 586-595 (2010).
26. P. G. Ashton-Rickardt, Serine protease inhibitors and cytotoxic T lymphocytes. Immunol Rev 235, 147-158 (2010).
27. J. Cooley, T. K. Takayama, S. D. Shapiro, N. M. Schechter, E. Remold-O'Donnell, The serpin MNEI inhibits elastase-like and chymotrypsin-like serine proteases through efficient reactions at two active sites. Biochemistry 40, 15762-15770 (2001).
28. E. Remold-O'Donnell, J. Chin, M. Alberts, Sequence and molecular characterization of human monocyte/neutrophil elastase inhibitor. Proc Natl Acad Sci USA 89, 5635-5639 (1992).
29. W. Zeng, G. A. Silverman, E. Remold-O'Donnell, Structure and sequence of human M/NEI (monocyte/neutrophil elastase inhibitor), an Ov-serpin family gene. Gene 213, 179-187 (1998).
30. C. Benarafa, J. Cooley, W. Zeng, P. I. Bird, E. Remold-O'Donnell, Characterization of four murine homologs of the human ov-serpin monocyte neutrophil elastase inhibitor MNEI (SERPINB1). J Biol Chem 277, 42028-42033 (2002).
31. C. Benarafa, E. Remold-O'Donnell, The ovalbumin serpins revisited: perspective from the chicken genome of clade B serpin evolution in vertebrates. Proc Natl Acad Sci USA 102, 11367-11372 (2005).
32. C. Benarafa, T. E. LeCuyer, M. Baumann, J. M. Stolley, T. P. Cremona, E. Remold-O'Donnell, SerpinB1 protects the mature neutrophil reserve in the bone marrow. J Leukoc Biol 90, 21-29 (2011).
33. C. Benarafa, G. P. Priebe, E. Remold-O'Donnell, The neutrophil serine protease inhibitor serpinb1 preserves lung defense functions in Pseudomonas aeruginosa infection. J Exp Med 204, 1901-1909 (2007).
34. M. Baumann, C. T. Pham, C. Benarafa, SerpinB1 is critical for neutrophil survival through cell-autonomous inhibition of cathepsin G. Blood 121, 3900-3907, S3901-3906 (2013).
35. F. Loison, H. Zhu, K. Karatepe, A. Kasom, P. Liu, K. Ye, J. Zhou, S. Cao, H. Gong, D. E. Jenne, E. Remold-O'Donnell, Y. Xu, H. R. Luo, Proteinase 3-dependent caspase-3 cleavage modulates neutrophil death and inflammation. J Clin Invest 124, 4445-4458 (2014).
36. P. Zhao, L. Hou, K. Farley, M. S. Sundrud, E. Remold-O'Donnell, SerpinB1 regulates homeostatic expansion of IL-17+ gammadelta and CD4+ Th17 cells. J Leukoc Biol 95, 521-530 (2014).
37. H. Georgiev, I. Ravens, C. Benarafa, R. Forster, G. Bernhardt, Distinct gene expression patterns correlate with developmental and functional traits of iNKT subsets. Nat Commun 7, 13116 (2016).
38. L. Hou, J. Cooley, R. Swanson, P. C. Ong, R. N. Pike, M. Bogyo, S. T. Olson, E. Remold-O'Donnell, The protease cathepsin L regulates Th17 cell differentiation. J Autoimmun 65, 56-63 (2015).
39. C. Wu, N. Yosef, T. Thalhamer, C. Zhu, S. Xiao, Y. Kishi, A. Regev, V. K. Kuchroo, Induction of pathogenic TH17 cells by inducible salt-sensing kinase SGK1. Nature 496, 513-517 (2013).
40. F. Annunziato, L. Cosmi, V. Santarlasci, L. Maggi, F. Liotta, B. Mazzinghi, E. Parente, L. Fili, S. Ferri, F. Frosali, F. Giudici, P. Romagnani, P. Parronchi, F. Tonelli, E. Maggi, S. Romagnani, Phenotypic and functional features of human Th17 cells. J Exp Med 204, 1849-1861 (2007).
41. Y. Cao, B. A. Goods, K. Raddassi, G. T. Nepom, W. W. Kwok, J. C. Love, D. A. Hafler, Functional inflammatory profiles distinguish myelin-reactive T cells from patients with multiple sclerosis. Sci Transl Med 7, 287ra274 (2015).
42. H. K. Deng, D. Unutmaz, V. N. KewalRamani, D. R. Littman, Expression cloning of new receptors used by simian and human immunodeficiency viruses. Nature 388, 296-300 (1997).
43. Y. Lee, A. Awasthi, N. Yosef, F. J. Quintana, S. Xiao, A. Peters, C. Wu, M. Kleinewietfeld, S. Kunder, D. A. Hafler, R. A. Sobel, A. Regev, V. K. Kuchroo, Induction and molecular signature of pathogenic TH17 cells. Nat Immunol 13, 991-999 (2012).
44. J. V. Kim, N. Jiang, C. E. Tadokoro, L. Liu, R. M. Ransohoff, J. J. Lafaille, M. L. Dustin, Two-photon laser scanning microscopy imaging of intact spinal cord and cerebral cortex reveals requirement for CXCR6 and neuroinflammation in immune cell infiltration of cortical injury sites. J Immunol Methods 352, 89-100 (2010).
45. P. G. Ashton-Rickardt, An emerging role for Serine Protease Inhibitors in T lymphocyte immunity and beyond. Immunol Lett 152, 65-76 (2013).
46. P. Boya, G. Kroemer, Lysosomal membrane permeabilization in cell death. Oncogene 27, 6434-6451 (2008).
47. N. Zamzami, P. Marchetti, M. Castedo, C. Zanin, J. L. Vayssiere, P. X. Petit, G. Kroemer, Reduction in mitochondrial potential constitutes an early irreversible step of programmed lymphocyte death in vivo. J Exp Med 181, 1661-1672 (1995).
48. H. Johnson, L. Scorrano, S. J. Korsmeyer, T. J. Ley, Cell death induced by granzyme C. Blood 101, 3093-3101 (2003).
49. B. Becher, B. M. Segal, T(H)17 cytokines in autoimmune neuro-inflammation. Curr Opin Immunol 23, 707-712 (2011).
50. H. Kebir, I. Ifergan, J. I. Alvarez, M. Bernard, J. Poirier, N. Arbour, P. Duquette, A. Prat, Preferential recruitment of interferon-gamma-expressing TH17 cells in multiple sclerosis. Ann Neurol 66, 390-402 (2009).
51. A. Mazzoni, V. Santarlasci, L. Maggi, M. Capone, M. C. Rossi, V. Querci, R. De Palma, H. D. Chang, A. Thiel, R. Cimaz, F. Liotta, L. Cosmi, E. Maggi, A. Radbruch, S. Romagnani, J. Dong, F. Annunziato, Demethylation of the RORC2 and IL17A in human CD4+ T lymphocytes defines Th17 origin of nonclassic Th1 cells. J Immunol 194, 3116-3126 (2015).
52. C. H. Kim, E. J. Kunkel, J. Boisvert, B. Johnston, J. J. Campbell, M. C. Genovese, H. B. Greenberg, E. C. Butcher, Bonzo/CXCR6 expression defines type 1-polarized T-cell subsets with extralymphoid tissue homing potential. J Clin Invest 107, 595-601 (2001).
53. P. P. Ahem, C. Schiering, S. Buonocore, M. J. McGeachy, D. J. Cua, K. J. Maloy, F. Powrie, Interleukin-23 drives intestinal inflammation through direct activity on T cells. Immunity 33, 279-288 (2010).
54. Y. Mandai, D. Takahashi, K. Hase, Y. Obata, Y. Furusawa, M. Ebisawa, T. Nakagawa, T. Sato, T. Katsuno, Y. Saito, T. Shimaoka, O. Yokosuka, K. Yokote, H. Ohno, Distinct Roles for CXCR6(+) and CXCR6(−) CD4(+) T Cells in the Pathogenesis of Chronic Colitis. PLoS One 8, e65488 (2013).
55. L. M. Peeters, M. Vanheusden, V. Somers, B. Van Wijmeersch, P. Stinissen, B. Broux, N. Hellings, Cytotoxic CD4+ T Cells Drive Multiple Sclerosis Progression. Front Immunol 8, 1160 (2017).
56. W. J. Grossman, P. A. Revell, Z. H. Lu, H. Johnson, A. J. Bredemeyer, T. J. Ley, The orphan granzymes of humans and mice. Curr Opin Immunol 15, 544-552 (2003).
57. L. Wang, Q. Li, L. Wu, S. Liu, Y. Zhang, X. Yang, P. Zhu, H. Zhang, K. Zhang, J. Lou, P. Liu, L. Tong, F. Sun, Z. Fan, Identification of SERPINB1 as a physiological inhibitor of human granzyme H. J Immunol 190, 1319-1330 (2013).
58. K. Aden, A. Rehman, M. Falk-Paulsen, T. Secher, J. Kuiper, F. Tran, S. Pfeuffer, R. Sheibani-Tezerji, A. Breuer, A. Luzius, M. Jentzsch, R. Hasler, S. Billmann-Bom, O. Will, S. Lipinski, R. Bharti, T. Adolph, J. L. Iovanna, S. L. Kempster, R. S. Blumberg, S. Schreiber, B. Becher, M. Chamaillard, A. Kaser, P. Rosenstiel, Epithelial IL-23R Signaling Licenses Protective IL-22 Responses in Intestinal Inflammation. Cell Rep 16, 2208-2218 (2016).
59. M. Reers, T. W. Smith, L. B. Chen, J-aggregate formation of a carbocyanine as a quantitative fluorescent indicator of membrane potential. Biochemistry 30, 4480-4486 (1991).
60. D. Kaiserman, A. M. Buckle, P. Van Damme, J. A. Irving, R. H. Law, A. Y. Matthews, T. Bashtannyk-Puhalovich, C. Langendorf, P. Thompson, J. Vandekerckhove, K. Gevaert, J. C. Whisstock, P. I. Bird, Structure of granzyme C reveals an unusual mechanism of protease autoinhibition. Proc Natl Acad Sci USA 106, 5587-5592 (2009).

SEQUENCES

Nucleic acid sequence encoding CXCR6 (SEQ ID NO: 1).
atggcagagc atgattacca tgaagactat gggttcagca
gtttcaatga cagcagccag gaggagcatc aagacttcct gcagttcagc aaggtctttc
tgccctgcat gtacctggtg gtgtttgtct gtggtctggt ggggaactct ctggtgctgg
tcatatccat cttctaccat aagttgcaga gcctgacgga tgtgttcctg gtgaacctac
ccctggctga cctggtgttt gtctgcactc tgcccttctg ggcctatgca ggcatccatg
aatgggtgtt tggccaggtc atgtgcaaga gcctactggg catctacact attaacttct
acacgtccat gctcatcctc acctgcatca ctgtggatcg tttcattgta gtggttaagg
ccaccaaggc ctacaaccag caagccaaga ggatgacctg gggcaaggtc accagcttgc
tcatctgggt gatatccctg ctggtttcct tgccccaaat tatctatggc aatgtcttta
atctcgacaa gctcatatgt ggttaccatg acgaggcaat ttccactgtg gttcttgcca
cccagatgac actggggttc ttcttgccac tgctcaccat gattgtctgc tattcagtca
taatcaaaac actgcttcat gctggaggct tccagaagca cagatctcta aagatcatct
tcctggtgat ggctgtgttc ctgctgaccc agatgccctt caacctcatg aagttcatcc
gcagcacaca ctgggaatac tatgccatga ccagctttca ctacaccatc atggtgacag
aggccatcgc atacctgagg gcctgcctta accctgtgct ctatgccttt gtcagcctga
agtttcgaaa gaacttctgg aaacttgtga aggacattgg ttgcctccct taccttgggg
tctcacatca atggaaatct tctgaggaca attccaagac tttttctgcc tcccacaatg
tggaggccac cagcatgttc cagttatag
Nucleic acid sequence encoding SerpinB1 (SEQ ID NO: 2).
tgg agcagctgag ctcagcaaac acccgcttcg ccttggacct gttcctggcg
ttgagtgaga acaatccggc tggaaacatc ttcatctctc ccttcagcat ttcatctgct
atggccatgg tttttctggg gaccagaggt aacacggcag cacagctgtc caagactttc
catttcaaca cggttgaaga ggttcattca agattccaga gtctgaatgc tgatatcaac
aaacgtggag cgtcttatat tctgaaactt gctaatagat tatatggaga gaaaacttac
aatttccttc ctgagttctt ggtttcgact cagaaaacat atggtgctga cctggccagt
gtggattttc agcatgcctc tgaagatgca aggaagacca taaaccagtg ggtcaaagga
cagacagaag gaaaaattcc ggaactgttg gcttcgggca tggttgataa catgaccaaa
cttgtgctag taaatgccat ctatttcaag ggaaactgga aggataaatt catgaaagaa
gccacgacga atgcaccatt cagattgaat aagaaagaca gaaaaactgt gaaaatgatg
tatcagaaga aaaaatttgc atatggctac atcgaggacc ttaagtgccg tgtgctggaa
ctgccttacc aaggcgagga gctcagcatg gtcatcctgc tgccggatga cattgaggac
gagtccacgg gcctgaagaa gattgaggaa cagttgactt tggaaaagtt gcatgagtgg
actaaacctg agaatctcga tttcattgaa gttaatgtca gcttgcccag gttcaaactg
gaagagagtt acactctcaa ctccgacctc gcccgcctag gtgtgcagga tctctttaac
agtagcaagg ctgatctgtc tggcatgtca ggagccagag atatttttat atcaaaaatt
gtccacaagt catttgtgga agtgaatgaa gagggaacag aggcggcagc tgccacagca
ggcatcgcaa ctttctgcat gttgatgccc gaagaaaatt tcactgccga ccatccattc
cttttcttta ttcggcataa ttcctcaggt agcatcctat tcttggggag attttcttcc
ccttag
Polypeptide sequence encoding CXCR6 (SEQ ID NO: 3)

| | |
|---|---|
| 1 | maehdyhedy gfssfndssq eehqdflqfs kvflpcmylv vfvcglvgns lvlvisifyh |
| 61 | klqsltdvfl vnlpladlvf vctlpfwaya gihewvfgqv mcksllgiyt infytsmlil |
| 121 | tcitvdrfiv vvkatkaynq qakrmtwgkv tslliwvisl lvslpqiiyg nvfnldklic |
| 181 | gyhdeaistv vlatqmtlgf flplltmivc ysviiktllh aggfqkhrsl kliflvmavf |
| 241 | lltqmpfnlm kfirsthwey yamtsfhyti mvteaiaylr aclnpvlyaf vslkfrknfw |
| 301 | klvkdigclp ylgvshqwks sednsktfsa shnveatsmf ql |

Polypeptide sequence encoding SerpinB1 (SEQ ID NO: 4)

| | |
|---|---|
| 1 | meqlssantr faldlflals ennpagnifi spfsissama mvflgtrgnt aaqlsktfhf |
| 61 | ntveevhsrf qslnadinkr gasyilklan rlygektynf lpeflvstqk tygadlasvd |
| 121 | fqhasedark tinqwvkgqt egkipellas gmvdnmtklv lvnaiyfkgn wkdkfmkeat |
| 181 | tnapfrlnkk drktvkmmyq kkkfaygyie dlkcrvlelp yqgeelsmvi llpddiedes |
| 241 | tglkkieeql tleklhewtk penldfievn vslprfklee sytlnsdlar lgvqdlfnss |
| 301 | kadlsgmsga rdifiskivh ksfvevneeg teaaaatagi atfcmlmpee nftadhpflf |
| 361 | firhnssgsi lflgrfssp |

Polypeptide sequence encoding SerpinB1 X1 (SEQ ID NO: 5)

| | |
|---|---|
| 1 | meqlssantr faldlflals ennpagnifi spfsissama mvflgtrgnt aaqlsktfhf |
| 61 | ntveevhsrf qslnadinkr gasyilklan rlygektynf lpeflvstqk tygadlasvd |
| 121 | fqhasedark tinqwvkgqt egkipellas gmvdnmtklv lvnaiyfkgn wkdkfmkeat |
| 181 | tnapfrlnkk drktvkmmyq kkkfaygyie dlkcrvlelp yqgeelsmvi llpddiedes |
| 241 | tglkkieeql tleklhewtk penldfievn vslprfklee sytlnsdlar lgvqdlfnss |
| 301 | kadlsgmsga rdifiskivh ksfvevneeg teaaaatagi atfcmlmpee nftadhpflf |
| 361 | firhnssgsi lflgrfssp |

Polypeptide sequence encoding SerpinB1 X2 (SEQ ID NO: 6)

| | |
|---|---|
| 1 | mdslhgktfh fntveevhsr fqslnadink rgasyilkla nrlygektyn flpeflvstq |
| 61 | ktygadlasv dfqhasedar ktinqwvkgq tegkipella sgmvdnmtkl vlvnaiyfkg |
| 121 | nwkdkfmkea ttnapfrlnk kdrktvkmmy qkkkfaygyi edlkcrvlel pyqgeelsmv |
| 181 | illpddiede stglkkieeq ltleklhewt kpenldfiev nvslprfkle esytlnsdla |
| 241 | rlgvqdlfns skadlsgmsg ardifiskiv hksfvevnee gteaaaatag iatfcmlmpe |
| 301 | enftadhpfl ffirhnssgs ilflgrfssp |

Polypeptide sequence encoding mouse CXCR6 (SEQ ID NO: 7)

| | |
|---|---|
| 1 | mddghqesal ydghyegdfw lfnnssdnsq enkrflkfke vflpcvylvv fvfgllgnsl |
| 61 | vliiyifyqk lrtltdvfll nlpladlvfv ctlpfwayag tyewvfgtvm cktlrgmytm |
| 121 | nfyvsmltlt citvdrfivv vqatkafnrq akwkiwgqvi clliwvvsll vslpgiiygh |
| 181 | vqdidklicq yhseeistmv lviqmtlgff lplltmilcy sgiiktllha rnfqkhkslk |
| 241 | iiflvvavfl ltqtpfnlam liqstsweyy titsfkyaiv vteaiayfra clnpvlyafv |
| 301 | glkfrknvwk lmkdigclsh lgvssqwkss edssktcsas hnvettsmfq l |

Polypeptide sequence encoding mouse SerpinB1a (SEQ ID NO: 8)

| | |
|---|---|
| 1 | meqlssantl falelfqtln essptgniff spfsissala mvilgakgst aaqlsktfhf |
| 61 | dsvedihsrf qslnaevskr gashtlklan rlygektynf lpeylastqk mygadlapvd |
| 121 | flhasedark einqwvkgqt egkipellsv gvvdsmtklv lvnaiyfkgm weekfmtedt |
| 181 | tdapfrlskk dtktvkmmyq kkkfpfgyis dlkckvlemp yqggelsmvi llpkdiedes |
| 241 | tglkkiekqi tlekllewtk renlefidvh vklprfkiee sytlnsnlgr lgvqdlfsss |
| 301 | kadlsgmsgs rdlfiskivh ksfvevneeg teaaaatggi atfcmllpee eftvdhpfif |
| 361 | firhnptsnv lflgrvcsp |

1. A method for treating an autoimmune disease, comprising administering to a subject having an autoimmune disease an agent that targets CXCR6;
wherein targeting CXCR6 results in the depletion of a cell expressing CXCR6 or population thereof.

2. (canceled)

3. The method of claim 1, wherein the cell population is depleted by at least 10%, at least 20%, at least 30%, at least 40%, at least 50%, at least 60%, at least 70%, at least 80%, at least 90% or more as compared to an appropriate control.

4. The method of claim 1, wherein the cell population is a T cell, T helper cell, Th17 cell, or Th17-derived cell population.

5. The method of claim 1, wherein the agent that targets CXCR6 is linked to at least a second agent or at least a toxin.

6. The method of claim 1, wherein the autoimmune disease is selected from the list consisting of Rheumatoid arthritis, Crohn's disease, lupus, celiac disease, Sjogren's syndrome, polymyalgia rheumatic, multiple sclerosis, ankylosing spondylitis, type 1 diabetes, alopecia areata, vasculitis, autoimmune uveitis, juvenile idiopathic arthritis, and temporal arteritis.

7. The method of claim 1, wherein the autoimmune disease is multiple sclerosis.

8. The method of claim 1, wherein the subject is human.

9. The method of claim 1, wherein the agent that targets CXCR6 is selected from the group consisting of a small molecule, an antibody, and a peptide.

10. (canceled)

11. The method of claim 9, wherein the antibody is a depleting antibody.

12.-14. (canceled)

15. A method for selecting a population of T cells, Th17 cells or Th17-derived cells, the method comprising measuring the level of CXCR6 in a population of candidate cells, and selecting cells which exhibit expression of CXCR6.

16. The method of claim 15, wherein the level of CXCR6 is increased by at least 2-fold, at least 3-fold, at least 4-fold, at least 5-fold, at least 6-fold, at least 7-fold, at least 8-fold, at least 9-fold, at least 10-fold, or more as compared to a reference level.

17. A method of treating an autoimmune disease, the method comprising:
(a) receiving the results of an assay that indicate an increase in the levels of CXCR6 in a biological sample from a subject compared with an appropriate control; and
(b) administering to the subject an agent that target CXCR6.

18. The method of claim 17, wherein the assay is flow cytometry, reverse transcription-polymerase chain reaction (RT-PCR), RNA sequencing, or immunohistochemistry.

19. The method of claim 17, wherein the subject is suspected of having, or has an autoimmune disease.

20. (canceled)

21. The method of claim 17, further comprising, detecting the levels of one or more of: perforin-A, granzyme A (GzmA), GzmC, interleukin-17 (IL-17), IL-6, IL-21, IL-23, interleukin-23 receptor (IL-23R), IL-7Rα and IL-1R1, interferon gamma (IFNγ), RAR Related Orphan Receptor C (Rorc), and granulocyte-macrophage colony-stimulating factor (GM-CSF) in the subject.

22. The method of claim 17, further comprising, detecting leukocyte accumulation in the spinal cord.

23.-40. (canceled)

41. The method of claim 1, wherein the cell is a differentiated T cell.

42. The method of claim 5, wherein the toxin is anti-microtubule agent DM-1, a derivative of Maytansine, or monomethyl auristatin E (MMAE).


SEQUENCE LISTING (76 sequences)

SEQ ID NO: 1 - DNA, 1029 bp, Homo sapiens
  source 1..1029: mol_type = genomic DNA
atggcagagcatgattaccatgaagactatgggttcagcagtttcaatgacagcagccaggaggagcatcaagacttcctgcagttcagcaaggtctttctgccctgcatgtacctggtggtgtttgtctgtggtctggtggggaactctctggtgctggtcatatccatcttctaccataagttgcagagcctgacggatgtgttcctggtgaacctacccctggctgacctggtgtttgtctgcactctgcccttctgggcctatgcaggcatccatgaatgggtgtttggccaggtcatgtgcaagagcctactgggcatctacactattaacttctacacgtccatgctcatcctcacctgcatcactgtggatcgtttcattgtagtggttaaggccaccaaggcctacaaccagcaagccaagaggatgacctggggcaaggtcaccagcttgctcatctgggtgatatccctgctggtttccttgccccaaattatctatggcaatgtctttaatctcgacaagctcatatgtggttaccatgacgaggcaatttccactgtggttcttgccacccagatgacactggggttcttcttgccactgctcaccatgattgtctgctattcagtcataatcaaaacactgcttcatgctggaggcttccagaagcacagatctctaaagatcatcttcctggtgatggctgtgttcctgctgacccagatgcccttcaacctcatgaagttcatccgcagcacacactgggaatactatgccatgaccagctttcactacaccatcatggtgacagaggccatcgcatacctgagggcctgccttaaccctgtgctctatgcctttgtcagcctgaagtttcgaaagaacttctggaaacttgtgaaggacattggttgcctcccttaccttggggtctcacatcaatggaaatcttctgaggacaattccaagactttttctgcctcccacaatgtggaggccaccagcatgttccagttatag

SEQ ID NO: 2 - DNA, 1139 bp, Homo sapiens
  source 1..1139: mol_type = genomic DNA
tggagcagctgagctcagcaaacacccgcttcgccttggacctgttcctggcgttgagtgagaacaatccggctggaaacatcttcatctctcccttcagcatttcatctgctatggccatggtttttctggggaccagaggtaacacggcagcacagctgtccaagactttccatttcaacacggttgaagaggttcattcaagattccagagtctgaatgctgatatcaacaaacgtggagcgtcttatattctgaaacttgctaatagattatatggagagaaaacttacaatttccttcctgagttcttggtttcgactcagaaaacatatggtgctgacctggccagtgtggattttcagcatgcctctgaagatgcaaggaagaccataaaccagtgggtcaaaggacagacagaaggaaaaattccggaactgttggcttcgggcatggttgataacatgaccaaacttgtgctagtaaatgccatctatttcaagggaaactggaaggataaattcatgaaagaagccacgacgaatgcaccattcagattgaataagaaagacagaaaaactgtgaaaatgatgtatcagaagaaaaaatttgcatatggctacatcgaggaccttaagtgccgtgtgctggaactgccttaccaaggcgaggagctcagcatggtcatcctgctgccggatgacattgaggacgagtccacgggcctgaagaagattgaggaacagttgactttggaaaagttgcatgagtggactaaacctgagaatctcgatttcattgaagttaatgtcagcttgcccaggttcaaactggaagagagttacactctcaactccgacctcgcccgcctaggtgtgcaggatctctttaacagtagcaaggctgatctgtctggcatgtcaggagccagagatatttttatatcaaaaattgtccacaagtcatttgtggaagtgaatgaagagggaacagaggcggcagctgccacagcaggcatcgcaactttctgcatgttgatgcccgaagaaaatttcactgccgaccatccattccttttctttattcggcataattcctcaggtagcatcctattcttggggagattttcttccccttag

SEQ ID NO: 3 - protein, 342 aa, Homo sapiens
MAEHDYHEDYGFSSFNDSSQEEHQDFLQFSKVFLPCMYLVVFVCGLVGNSLVLVISIFYHKLQSLTDVFLVNLPLADLVFVCTLPFWAYAGIHEWVFGQVMCKSLLGIYTINFYTSMLILTCITVDRFIVVVKATKAYNQQAKRMTWGKVTSLLIWVISLLVSLPQIIYGNVFNLDKLICGYHDEAISTVVLATQMTLGFFLPLLTMIVCYSVIIKTLLHAGGFQKHRSLKIIFLVMAVFLLTQMPFNLMKFIRSTHWEYYAMTSFHYTIMVTEAIAYLRACLNPVLYAFVSLKFRKNFWKLVKDIGCLPYLGVSHQWKSSEDNSKTFSASHNVEATSMFQL

SEQ ID NO: 4 - protein, 379 aa, Homo sapiens
MEQLSSANTRFALDLFLALSENNPAGNIFISPFSISSAMAMVFLGTRGNTAAQLSKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFCMLMPEENFTADHPFLFFIRHNSSGSILFLGRFSSP

SEQ ID NO: 5 - protein, 379 aa, Homo sapiens; identical to SEQ ID NO: 4

SEQ ID NO: 6 - protein, 330 aa, Homo sapiens
MDSLHGKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFCMLMPEENFTADHPFLFFIRHNSSGSILFLGRFSSP

SEQ ID NO: 7 - protein, 351 aa, Mus musculus
MDDGHQESALYDGHYEGDFWLFNNSSDNSQENKRFLKFKEVFLPCVYLVVFVFGLLGNSLVLIIYIFYQKLRTLTDVFLLNLPLADLVFVCTLPFWAYAGTYEWVFGTVMCKTLRGMYTMNFYVSMLTLTCITVDRFIVVVQATKAFNRQAKWKIWGQVICLLIWVVSLLVSLPQIIYGHVQDIDKLICQYHSEEISTMVLVIQMTLGFFLPLLTMILCYSGIIKTLLHARNFQKHKSLKIIFLVVAVFLLTQTPFNLAMLIQSTSWEYYTITSFKYAIVVTEAIAYFRACLNPVLYAFVGLKFRKNVWKLMKDIGCLSHLGVSSQWKSSEDSSKTCSASHNVETTSMFQL

SEQ ID NO: 8 - protein, 379 aa, Mus musculus
MEQLSSANTLFALELFQTLNESSPTGNIFFSPFSISSALAMVILGAKGSTAAQLSKTFHFDSVEDIHSRFQSLNAEVSKRGASHTLKLANRLYGEKTYNFLPEYLASTQKMYGADLAPVDFLHASEDARKEINQWVKGQTEGKIPELLSVGVVDSMTKLVLVNAIYFKGMWEEKFMTEDTTDAPFRLSKKDTKTVKMMYQKKKFPFGYISDLKCKVLEMPYQGGELSMVILLPKDIEDESTGLKKIEKQITLEKLLEWTKRENLEFIDVHVKLPRFKIEESYTLNSNLGRLGVQDLFSSSKADLSGMSGSRDLFISKIVHKSFVEVNEEGTEAAAATGGIATFCMLLPEEEFTVDHPFIFFIRHNPTSNVLFLGRVCSP

SEQ ID NO: 9 - DNA, 20 bp, synthetic construct
  misc_feature 1..20: note = Description of Artificial Sequence: Synthetic primer
  source 1..20: mol_type = other DNA
ggctgtattcccctccatcg

SEQ ID NO: 10 - DNA, 22 bp, synthetic construct
  misc_feature 1..22: note = Description of Artificial Sequence: Synthetic primer
  source 1..22: mol_type = other DNA
agcttctgttatgaggctcacc

SEQ ID NO: 11 - DNA, 22 bp, synthetic construct
  misc_feature 1..22: note = Description of Artificial Sequence: Synthetic primer
  source 1..22: mol_type = other DNA
cagttcggctataacactggtg

SEQ ID NO: 12 - DNA, 21 bp, synthetic construct
  misc_feature 1..21: note = Description of Artificial Sequence: Synthetic primer
  source 1..21: mol_type = other DNA
tgatcgcctgcttattcacgg

SEQ ID NO: 13 - DNA, 21 bp, synthetic construct
  misc_feature 1..21: note = Description of Artificial Sequence: Synthetic primer
  source 1..21: mol_type = other DNA
cgcctatccgaagtacctgga

SEQ ID NO: 14 - DNA, 23 bp, synthetic construct
  misc_feature 1..23: note = Description of Artificial Sequence: Synthetic primer
  source 1..23: mol_type = other DNA
cattgtcaagtacagtccacact

SEQ ID NO: 15 - DNA, 21 bp, synthetic construct
  misc_feature 1..21: note = Description of Artificial Sequence: Synthetic primer
  source 1..21: mol_type = other DNA
atgggcggaatggtctctttc

SEQ ID NO: 16 - DNA, 21 bp, synthetic construct
  misc_feature 1..21: note = Description of Artificial Sequence: Synthetic primer
  source 1..21: mol_type = other DNA
atgcccctcaacgtgaacttc

SEQ ID NO: 17 - DNA, 21 bp, synthetic construct
  misc_feature 1..21: note = Description of Artificial Sequence: Synthetic primer
  source 1..21: mol_type = other DNA
gcgaggaatgtggtgtgtttg

SEQ ID NO: 18 - DNA, 19 bp, synthetic construct
  misc_feature 1..19: note = Description of Artificial Sequence: Synthetic primer
  source 1..19: mol_type = other DNA
gcgcctccatcagtctttg

SEQ ID NO: 19 - DNA, 23 bp, synthetic construct
  misc_feature 1..23: note = Description of Artificial Sequence: Synthetic primer
  source 1..23: mol_type = other DNA
ttaaaaacctggatcggaaccaa

SEQ ID NO: 20 - DNA, 23 bp, synthetic construct
  misc_feature 1..23: note = Description of Artificial Sequence: Synthetic primer
  source 1..23: mol_type = other DNA
ttctctgtaccatgacactctgc

SEQ ID NO: 21 - DNA, 23 bp, synthetic construct
  misc_feature 1..23: note = Description of Artificial Sequence: Synthetic primer
  source 1..23: mol_type = other DNA
ttcctgctgtttctcttacacct

SEQ ID NO: 22 - DNA, 20 bp, synthetic construct
  misc_feature 1..20: note = Description of Artificial Sequence: Synthetic primer
  source 1..20: mol_type = other DNA
gctgctttgcctacctctcc

SEQ ID NO: 23 - DNA, 21 bp, synthetic construct
  misc_feature 1..21: note = Description of Artificial Sequence: Synthetic primer
  source 1..21: mol_type = other DNA
gcctctcgtacatacagacgc

SEQ ID NO: 24 - DNA, 23 bp, synthetic construct
  misc_feature 1..23: note = Description of Artificial Sequence: Synthetic primer
  source 1..23: mol_type = other DNA
tgagcaggatggagaattacagg

SEQ ID NO: 25 - DNA, 23 bp, synthetic construct
  misc_feature 1..23: note = Description of Artificial Sequence: Synthetic primer
  source 1..23: mol_type = other DNA
ttcagatgggcatgaatgtttct

SEQ ID NO: 26 - DNA, 21 bp, synthetic construct
  misc_feature 1..21: note = Description of Artificial Sequence: Synthetic primer
  source 1..21: mol_type = other DNA
cctgggcaacattatggtggt

SEQ ID NO: 27 - DNA, 22 bp, synthetic construct
  misc_feature 1..22: note = Description of Artificial Sequence: Synthetic primer
  source 1..22: mol_type = other DNA
gcaactgttcctgaactcaact

SEQ ID NO: 28 - DNA, 21 bp, synthetic construct
  misc_feature 1..21: note = Description of Artificial Sequence: Synthetic primer
  source 1..21: mol_type = other DNA
tttaactcccttggcgcaaaa

SEQ ID NO: 29 - DNA, 21 bp, synthetic construct
  misc_feature 1..21: note = Description of Artificial Sequence: Synthetic primer
  source 1..21: mol_type = other DNA
tcatcattgacctcgtggccc

SEQ ID NO: 30 - DNA, 27 bp, synthetic construct
  misc_feature 1..27: note = Description of Artificial Sequence: Synthetic primer
  source 1..27: mol_type = other DNA
gtgggatccctgatggctgtcctgcag

SEQ ID NO: 31 - DNA, 21 bp, synthetic construct
  misc_feature 1..21: note = Description of Artificial Sequence: Synthetic primer
  source 1..21: mol_type = other DNA
gctcttactgactggcatgag

SEQ ID NO: 32 - DNA, 21 bp, synthetic construct
  misc_feature 1..21: note = Description of Artificial Sequence: Synthetic primer
  source 1..21: mol_type = other DNA
ggccttggaagcatgtagagg

SEQ ID NO: 33 - DNA, 21 bp, synthetic construct
  misc_feature 1..21: note = Description of Artificial Sequence: Synthetic primer
  source 1..21: mol_type = other DNA
atgaacgctacacactgcatc

SEQ ID NO: 34 - DNA, 20 bp, synthetic construct
  misc_feature 1..20: note = Description of Artificial Sequence: Synthetic primer
  source 1..20: mol_type = other DNA
gcggacgatcactccttctg

SEQ ID NO: 35 - DNA, 22 bp, synthetic construct
  misc_feature 1..22: note = Description of Artificial Sequence: Synthetic primer
  source 1..22: mol_type = other DNA
ccactggatgcgccaggaagtt

SEQ ID NO: 36 - DNA, 19 bp, synthetic construct
  misc_feature 1..19: note = Description of Artificial Sequence: Synthetic primer
  source 1..19: mol_type = other DNA
cgcggagcagacacactta

SEQ ID NO: 37 - DNA, 21 bp, synthetic construct
  misc_feature 1..21: note = Description of Artificial Sequence: Synthetic primer
  source 1..21: mol_type = other DNA
gcagaggagataatcggaggc

SEQ ID NO: 38 - DNA, 19 bp, synthetic construct
  misc_feature 1..19: note = Description of Artificial Sequence: Synthetic primer
  source 1..19: mol_type = other DNA
agcacaagttcgtgccagg

SEQ ID NO: 39 - DNA, 19 bp, synthetic construct
  misc_feature 1..19: note = Description of Artificial Sequence: Synthetic primer
  source 1..19: mol_type = other DNA
tgctgcccactgtaacgtg

SEQ ID NO: 40 - DNA, 20 bp, synthetic construct
  misc_feature 1..20: note = Description of Artificial Sequence: Synthetic primer
  source 1..20: mol_type = other DNA
ccactctcgaccctacatgg

SEQ ID NO: 41 - DNA, 19 bp, synthetic construct
  misc_feature 1..19: note = Description of Artificial Sequence: Synthetic primer
  source 1..19: mol_type = other DNA
tcaggtggctccgtcgatt

SEQ ID NO: 42 - DNA, 21 bp, synthetic construct
  misc_feature 1..21: note = Description of Artificial Sequence: Synthetic primer
  source 1..21: mol_type = other DNA
acatccattcacgcttccaaa

SEQ ID NO: 43 - DNA, 22 bp, synthetic construct
  misc_feature 1..22: note = Description of Artificial Sequence: Synthetic primer
  source 1..22: mol_type = other DNA
ccagttggtaacaatgccatgt

SEQ ID NO: 44 - DNA, 22 bp, synthetic construct
  misc_feature 1..22: note = Description of Artificial Sequence: Synthetic primer
  source 1..22: mol_type = other DNA
tgacttgatgttcatcgtcctc

SEQ ID NO: 45 - DNA, 19 bp, synthetic construct
  misc_feature 1..19: note = Description of Artificial Sequence: Synthetic primer
  source 1..19: mol_type = other DNA
gcccccgacagagaagatg

SEQ ID NO: 46 - DNA, 21 bp, synthetic construct
  misc_feature 1..21: note = Description of Artificial Sequence: Synthetic primer
  source 1..21: mol_type = other DNA
aaccgcctagaaatctccaga

SEQ ID NO: 47 - DNA, 20 bp, synthetic construct
  misc_feature 1..20: note = Description of Artificial Sequence: Synthetic primer
  source 1..20: mol_type = other DNA
ccccgtgtagattcccatgc

SEQ ID NO: 48 - DNA, 23 bp, synthetic construct
  misc_feature 1..23: note = Description of Artificial Sequence: Synthetic primer
  source 1..23: mol_type = other DNA
ttccaattactcggtttttggga

SEQ ID NO: 49 - DNA, 21 bp, synthetic construct
  misc_feature 1..21: note = Description of Artificial Sequence: Synthetic primer
  source 1..21: mol_type = other DNA
tggggaccttgtcttcatcat

SEQ ID NO: 50 - DNA, 21 bp, synthetic construct
  misc_feature 1..21: note = Description of Artificial Sequence: Synthetic primer
  source 1..21: mol_type = other DNA
cgcaacataggatggagagca

SEQ ID NO: 51 - DNA, 21 bp, synthetic construct
  misc_feature 1..21: note = Description of Artificial Sequence: Synthetic primer
  source 1..21: mol_type = other DNA
tttaggcactgcactcccatc

SEQ ID NO: 52 - DNA, 21 bp, synthetic construct
  misc_feature 1..21: note = Description of Artificial Sequence: Synthetic primer
  source 1..21: mol_type = other DNA
attcatctgtgcggtacgctc

SEQ ID NO: 53 - DNA, 23 bp, synthetic construct
  misc_feature 1..23: note = Description of Artificial Sequence: Synthetic primer
  source 1..23: mol_type = other DNA
gcattagcttcagatttacgggt

SEQ ID NO: 54 - DNA, 21 bp, synthetic construct
  misc_feature 1..21: note = Description of Artificial Sequence: Synthetic primer
  source 1..21: mol_type = other DNA
cgtggaatcttccggctgtag

SEQ ID NO: 55 - DNA, 21 bp, synthetic construct
  misc_feature 1..21: note = Description of Artificial Sequence: Synthetic primer
  source 1..21: mol_type = other DNA
ctgtctgcctcttttggtcag

SEQ ID NO: 56 - DNA, 21 bp, synthetic construct
  misc_feature 1..21: note = Description of Artificial Sequence: Synthetic primer
  source 1..21: mol_type = other DNA
tcgagtgacaaacacgactgc

SEQ ID NO: 57 - DNA, 21 bp, synthetic construct
  misc_feature 1..21: note = Description of Artificial Sequence: Synthetic primer
  source 1..21: mol_type = other DNA
ccagttctgctttggatcagc

SEQ ID NO: 58 - DNA, 23 bp, synthetic construct
  misc_feature 1..23: note = Description of Artificial Sequence: Synthetic primer
  source 1..23: mol_type = other DNA
gtccaagttcatcttctaggcac

SEQ ID NO: 59 - DNA, 23 bp, synthetic construct
  misc_feature 1..23: note = Description of Artificial Sequence: Synthetic primer
  source 1..23: mol_type = other DNA
ccaaatccgagctgttgttctat

SEQ ID NO: 60 - DNA, 21 bp, synthetic construct
  misc_feature 1..21: note = Description of Artificial Sequence: Synthetic primer
  source 1..21: mol_type = other DNA
cagaacggtagggtgaggaca

SEQ ID NO: 61 - DNA, 21 bp, synthetic construct
  misc_feature 1..21: note = Description of Artificial Sequence: Synthetic primer
  source 1..21: mol_type = other DNA
atcttttggggtccgtcaact

SEQ ID NO: 62 - DNA, 20 bp, synthetic construct
  misc_feature 1..20: note = Description of Artificial Sequence: Synthetic primer
  source 1..20: mol_type = other DNA
ctttccctccgcattgacac

SEQ ID NO: 63 - DNA, 24 bp, synthetic construct
  misc_feature 1..24: note = Description of Artificial Sequence: Synthetic primer
  source 1..24: mol_type = other DNA
atcgtacttctccacttgcaatcc

SEQ ID NO: 64 - DNA, 28 bp, synthetic construct
  misc_feature 1..28: note = Description of Artificial Sequence: Synthetic primer
  source 1..28: mol_type = other DNA
agcgaattctcgctcagactgcaagcat

SEQ ID NO: 65 - DNA, 20 bp, synthetic construct
  misc_feature 1..20: note = Description of Artificial Sequence: Synthetic primer
  source 1..20: mol_type = other DNA
cgcagctctaggagcatgtg

SEQ ID NO: 66 - DNA, 23 bp, synthetic construct
  misc_feature 1..23: note = Description of Artificial Sequence: Synthetic primer
  source 1..23: mol_type = other DNA
ggagaactcgttagagacgactt

SEQ ID NO: 67 - DNA, 21 bp, synthetic construct
  misc_feature 1..21: note = Description of Artificial Sequence: Synthetic primer
  source 1..21: mol_type = other DNA
ccatccttttgccagttcctc

SEQ ID NO: 68 - DNA, 23 bp, synthetic construct
  misc_feature 1..23: note = Description of Artificial Sequence: Synthetic primer
  source 1..23: mol_type = other DNA
agccccacatatttgaaattcca

SEQ ID NO: 69 - DNA, 22 bp, synthetic construct
  misc_feature 1..22: note = Description of Artificial Sequence: Synthetic primer
  source 1..22: mol_type = other DNA
ttcacctccacgatgtgcagcc

SEQ ID NO: 70 - DNA, 20 bp, synthetic construct
  misc_feature 1..20: note = Description of Artificial Sequence: Synthetic primer
  source 1..20: mol_type = other DNA
ccctggacctctgttttggc

SEQ ID NO: 71 - DNA, 21 bp, synthetic construct
  misc_feature 1..21: note = Description of Artificial Sequence: Synthetic primer
  source 1..21: mol_type = other DNA
gcacgaatttgtctcgaacca

SEQ ID NO: 72 - DNA, 23 bp, synthetic construct
  misc_feature 1..23: note = Description of Artificial Sequence: Synthetic primer
  source 1..23: mol_type = other DNA
gcgtctctcattagggagttttt

SEQ ID NO: 73 - DNA, 22 bp, synthetic construct
  misc_feature 1..22: note = Description of Artificial Sequence: Synthetic primer
  source 1..22: mol_type = other DNA
ggtaggtgaaggatagccacat

SEQ ID NO: 74 - DNA, 22 bp, synthetic construct
  misc_feature 1..22: note = Description of Artificial Sequence: Synthetic primer
  source 1..22: mol_type = other DNA
ggcccccaaagtgacatttatt

SEQ ID NO: 75 - DNA, 22 bp, synthetic construct
  misc_feature 1..22: note = Description of Artificial Sequence: Synthetic primer
  source 1..22: mol_type = other DNA
ggcatgtccattgtgtactctt

SEQ ID NO: 76 - DNA, 21 bp, synthetic construct
  misc_feature 1..21: note = Description of Artificial Sequence: Synthetic primer
  source 1..21: mol_type = other DNA
ggccaagtcagcaccatacat